(19) United States
(12) Patent Application Publication
(10) Pub. No.: US 2011/0160772 A1
(43) Pub. Date: Jun. 30, 2011

(54) SYSTEMS AND METHODS FOR PERFORMING SPINAL FUSION

(76) Inventors: Gregory B. Arcenio, Redwood City, CA (US); Anthony J. Recupero, Pleasanton, CA (US); Michael P. Wallace, Pleasanton, CA (US); Glenn R. Davis, Boulder Creek, CA (US)

(21) Appl. No.: 12/980,165

(22) Filed: Dec. 28, 2010

Related U.S. Application Data
(60) Provisional application No. 61/290,458, filed on Dec. 28, 2009.
(60) Provisional application No. 61/314,557, filed on Mar. 16, 2010.
(60) Provisional application No. 61/318,941, filed on Mar. 30, 2010.
(60) Provisional application No. 61/329,563, filed on Apr. 30, 2010.
(60) Provisional application No. 61/350,600, filed on Jun. 2, 2010.
(60) Provisional application No. 61/356,557, filed on Jun. 19, 2010.
(60) Provisional application No. 61/357,529, filed on Jun. 23, 2010.
(60) Provisional application No. 61/366,137, filed on Jul. 20, 2010.
(60) Provisional application No. 61/366,487, filed on Jul. 21, 2010.
(60) Provisional application No. 61/370,456, filed on Aug. 4, 2010.
(60) Provisional application No. 61/372,525, filed on Aug. 11, 2010.
(60) Provisional application No. 61/372,591, filed on Aug. 11, 2010.
(60) Provisional application No. 61/392,192, filed on Oct. 12, 2010.

(51) Int. Cl. A61B 17/70; A61B 17/56; A61B 17/88

(52) U.S. Cl. 606/248; 606/86 A; 606/279

(57) ABSTRACT

Described herein are methods, devices and systems for performing an interspinous fusion, in particular for performing an interspinous fusion unilaterally. In general an interspinous fusion system may include a first fixation plate configured to couple to a first lateral side of a spinous process, a rod extending from the first fixation plate at a joint such that the rod is pivotable with respect to the first fixation plate, and a second fixation plate configured to couple to a second lateral side of a spinous process opposite from the first fixation plate. In general, a method of performing an interspinous fusion unilaterally may include the steps of placing a first fixation plate, having a rod extending from the fixation plate, between two adjacent spinous processes from a fist lateral side of the spinous processes, pivoting the rod with respect to the first fixation plate such that the plate abuts the second, opposite, lateral side of at least one of the spinous processes, and placing a second fixation plate such that it abuts the fist lateral side of at least one of the spinous processes.

### CROSS REFERENCE TO RELATED APPLICATIONS

This patent application claims priority to U.S. Provisional Application No. 61/290,458, titled “SYSTEMS AND METHODS FOR PERFORMING SPINAL FUSION”, filed on Dec. 28, 2009; U.S. Provisional Application No. 61/314,557, titled “TISSUE REMOVAL AND IMPLANT DELIVERY DEVICES, SYSTEMS, AND METHODS”, filed on Mar. 16, 2010; U.S. Provisional Application No. 61/318,941, titled “TISSUE REMOVAL AND IMPLANT DELIVERY DEVICES, SYSTEMS, AND METHODS”, filed on Mar. 30, 2010; U.S. Provisional Application No. 61/329,563, titled “SYSTEMS AND METHODS FOR PERFORMING SPINAL FUSION”, filed on Apr. 30, 2010; U.S. Provisional Application No. 61/350,600, titled “SYSTEMS AND METHODS FOR PERFORMING SPINAL FUSION”, filed on Jun. 2, 2010; U.S. Provisional Application No. 61/356,557, titled “SYSTEMS AND METHODS FOR PERFORMING SPINAL FUSION”, filed on Jun. 19, 2010; U.S. Provisional Application No. 61/357,529, titled “SYSTEMS AND METHODS FOR PERFORMING SPINAL FUSION”, filed on Jun. 23, 2010; U.S. Provisional Application No. 61/366,137, titled “SYSTEMS AND METHODS FOR PERFORMING SPINAL FUSION”, filed on Jul. 20, 2010; U.S. Provisional Application No. 61/366,487, titled “SYSTEMS AND METHODS FOR PERFORMING SPINAL FUSION”, filed on Jul. 21, 2010; U.S. Provisional Application No. 61/370,456, titled “SYSTEMS AND METHODS FOR PERFORMING SPINAL FUSION”, filed on Aug. 4, 2010; U.S. Provisional Application No. 61/372,525, titled “SYSTEMS AND METHODS FOR PERFORMING SPINAL FUSION”, filed on Aug. 11, 2010; U.S. Provisional Application No. 61/372,591, titled “SYSTEMS AND METHODS FOR PERFORMING SPINAL FUSION”, filed on Aug. 11, 2010; and U.S. Provisional Application No. 61/392,192, titled “ACCESS AND TISSUE MODIFICATION SYSTEMS AND METHODS”, filed on Oct. 12, 2010. These provisional applications are each incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are methods, devices and systems for removing tissue and/or delivering an implant. In particular, described herein are interspinous fusion systems and methods for performing an interspinous fusion unilaterally.

### BACKGROUND OF THE INVENTION

Minimally invasive surgical techniques typically include accessing the tissue through a small opening or port into the body. Minimally invasive procedures may include laparoscopic devices and remote-control manipulation of instruments with indirect observation of the surgical field through an endoscope or similar device, and may be carried out through the skin or through a body cavity or anatomical opening. This may result in shorter hospital stays, or allow outpatient treatment.

Unfortunately, the use of minimally-invasive techniques has often required a loss in control of the treatment device or implant, as the treatment sites are often deep within the body, proving both difficult to access, as well as difficult to manipulate the device when the body region is minimally invasively accessed. In particular, finding leverage to position or manipulate minimally invasive devices once deployed has proven extremely difficult. For example, most procedures are performed from a single (minimally invasive) opening through the body to access the treatment site. Thus, any devices or implants delivered through this opening must be controlled externally through the single opening. As a result, complex and expensive tools have been created to allow manipulation of distally-positioned devices or implants within the body.

Even in variations of minimally invasive procedures in which a second access port is used, coordination of the two access ports at the target has proven difficult, particularly when one or more devices are inserted through different access ports and required to meet at an internal site. Such minimally invasive techniques often require the additional use of visualization devices to guide and/or confirm device position and operation.

Finally, manipulation of implants and devices using any of these minimally invasive techniques has also proven difficult. For example, when treating small or enclosed body regions such as joints, or regions surrounded by sensitive non-target tissue, manipulation of a device or implant within this space has been limited by the ability to control the distal end of the device from a proximal position. When a single access point is used, the device or implant must generally be ‘pushed’ into position within or along an access device. An elongate member (e.g., a cannula or guide) may be used, and the control of an implant or other device depends on the configuration of the access elongate member. Thus, the application of force by the implant or treatment device may depend on the application of force from the proximal end, at some distance from the distal end where the implant or treatment device is located. This may lead to undesirable and dangerous kinking, bending, and torqueing of the access device and/or implant.

Described herein are methods, devices and systems for spinal fusions. Some variations of the methods, devices and systems described herein may be configured for treating tissue by first placing a guidewire (or “pullwire”) in position within the body, and then using the guidewire to position, anchor and/or treat the tissue. In general, these methods and systems are “bimanual” procedures, in which the implant or tissue modification device is controlled within the body from one or more locations outside of the body, and by manipulating the implant/device from both the distal and proximal ends of the implant/device. The devices, methods and systems described herein may allow precise control and anchoring of one or more devices, and therefore precise treatment of tissue, and may address many of the issues raised above. Although the methods described herein may be particularly suitable for minimally invasive (e.g., percutaneous) treatment of tissue, they may also be used for open or semi-open treatments.

Spinal Fusion is the most used procedure in spinal surgery, and is usually combined with other procedures such as decompressive surgery. In fusion surgery an autograft, an allograft, or a xenograft loaded with a growth factor to induce bone formation, is placed between two bone surfaces to be fused. This section of the spine may also be anchored with screws, such as facet or pedicle screws. Over time the spine segments fuse together with the graft because of new bone growth, and become permanently immobile. There are many procedures and surgical approaches used to accomplish spinal fusion. The most common include Anterior Lumbar Interbody Fusion (ALIF), Posterior Lumbar Interbody Fusions (PLIFs), Minimally Invasive Posterior Lumbar Interbody Fusion (PLIF), eXtreme Lateral Interbody Fusion (XLIF) or Direct Lateral Interbody Fusion (DLIF), Intertransverse Lumbar Interbody Fusion (ILIF), and AxiaLIF.

For an Anterior Lumbar Interbody Fusion (ALIF) the patient is positioned on their back and the section of the spine to be fused is approach through wide incisions on the side of the abdomen. The abdominal organs are gently moved aside and horizontal holes are drilled in the problem discs of the spinal section to be fused. Bone graft material is packed into hollow fusion cages which are sized to fit into the newly drilled holes in the disc. The surgeon may also fix the vertebrae in place using metal screws or plates through the back of the spine to lock the two vertebrae and prevent them from moving. This protects the graft so it can heal better and faster.

In Posterior Lumbar Interbody Fusions (PLIFs), the surgical procedure is performed with the patient's back exposed with minimal abdominal pressure. The paraspinal muscles and tissues just under the skin, and those connected to the vertebrae are separated, and the surgeon can access the section of the spine to be treated. The surgeon can implant interbody cages, perform a decompression, and fix the spine with screws, such as facet or pedicle screws all through the incision through the patient's back. In an eXtreme Lateral Interbody Fusion (XLIF) or Direct Lateral Interbody Fusion (DLIF) the spine is also accessed with the patient lying face down, but rather than dissecting significant muscle tissue and ligaments, the lateral approach touts sparing muscle trauma, and with minimal risk of nerve damage. Retractors can help create a working space through which probes and other instruments can be safely guided, and surgery can be performed.

Transforaminal Lumbar Interbody Fusion (TLIF) is a version of PLIF wherein entry is gained through the foramina Minimally Invasive Posterior Lumbar Interbody Fusion (PLIF) is possible through the use of microsurgical ports and devices such as the METRx device from Medtronic. METRX is a small tube that is used to dilate tissues, thereby potentially eliminating the need for muscle splitting or cutting. The PLIF procedural steps of bone removal, discectomy, and bone graft/interbody implant placement are carried out through the METRx tubes. Once the bone grafts have been placed, the surgeon then may choose to stabilize the spine even further by fixing the area with rods and screws.

In the AxiaLIF technique, the lumbar spine is accessed through a minimally invasive opening adjacent to the sacral bone. This atraumatic tissue plane alleviates the need for the surgeon to cut through soft tissues like muscles and ligaments, thus lessening patient pain and the likelihood of complications.

There are many disadvantages to conventional spinal fusion surgery. For example, in conventional spinal fusion surgery, back muscles have to be moved from their spinal attachments allowing surgeon to place the rods, screws, grafts, and any other components necessary to complete the fusion. Often, incisions must be made and muscles must be dissected on both sides of the midline of the spine (bilateral incisions) to provide access for surgical instruments and implants to both sides of the spine. The dissection of the muscles produces the majority of the perioperative pain, reduced post-op activity, higher use of pain meds, delays to return to normal activity, to name just a few disadvantages. In some cases, the muscles can scar to one another and thereby lose independent function. Furthermore, in some cases there can be a loss of innervations and/or sensation. The methods, devices and systems described herein may address many of the disadvantages described above.

### SUMMARY OF THE INVENTION

Described herein are devices, systems and methods for treating tissue such as spinal tissue. In particular, described herein are methods, devices and systems for performing spinal fusions. For example, described herein are systems, devices and methods for performing interspinous fusions.

In some variations of the methods and systems for precisely placing and/or manipulating devices within the body, the methods and systems may be adapted to first position a guidewire through the body from a first location, around a curved pathway, and out of the body through a second location, so that the distal and proximal ends of the guidewire extend from the body, then pulling a device into position using the guidewire. The device to be positioned within the body is coupled to the proximal end of the guidewire, and the device is pulled into the body by pulling on the distal end of the guidewire that extends from the body. The device may be bimanually manipulated by pulling the guidewire distally, and an attachment to the device that extends proximally, allowing control of both the proximal and the distal ends. In this manner devices (and particularly implants such as innerspinous distracters, stimulating leads, and disc slings) may be positioned and/or manipulated within the body. Devices to modify tissue may also be positioned or manipulated so that a target tissue within the body is modified.

Devices and systems configured to be coupled to the proximal end of a pull guidewire (or “pullwire”) are also described. In general, a system for pulling an implant or tissue modification device into position as described herein may include a probe for positioning a guidewire into position, a guidewire/pullwire, a handle for the guidewire/pullwire, and a device having a distal end configured to couple to the pullwire and be pulled into position by the pullwire. The devices or implants may be adapted for use with the pullwire. For example the distal end of the devices/implants may be configured to releaseably secure to the proximal end of the pullwire. Furthermore, the devices may be adapted so that the connection with the guidewire is sufficient to withstand a substantial amount of pulling force that may be applied when positioning or manipulating the device(s).

For example, the general devices and methods described herein may be used to position and/or manipulate devices involved in the treatment of any of the following conditions: positioning/implanting stimulator leads (including anchoring them) within the body, and especially within the lateral recess or foramen; treatment of chronic total occlusions, including retrograde treatment (e.g., pull through); placement of pedicle screw(s); accessing a facet joint for fusion (e.g., posterior lateral gutters), implantation, etc.; spinal fusions, including percutaneously pulling in a rod between the screws; discectomy; remove or repair of disc herniation; pain management, including delivery of drug depot (e.g., ribbon, pod, electrodes, etc.), and particularly placement within spinal regions such as the facet joint; treatment of spine tumors (e.g., cage); insertion/implantation of stem cells; implantation of interlaminar wires; rapid laminectomy (e.g., in/out technique); treatment of distal clavicle, including shoulder impingement; treatment of entrapment Syndrome (e.g., carpel tunnel); removal of tumors, osteophites, around rib cage, ribs; thoracotomy; treatment of bone spurs; treatment of knees, including positioning/implanting drugs depots (e.g., steroids) and resurfacing of the joint; resurfacing of joints generally (spinal, etc.), including resurfacing of cartilage and preparation of joint for implant(s); removal of adipose (fat) tissue (e.g., liposuction); reconstructive surgeries (e.g., rhinoplasty, etc.); and the like.

Particular examples are described below, including devices adapted for use with these examples that illustrate methods of performing such treatments and therapies. For example, described herein are methods of performing inner spinous distraction. Inner spinous distraction may be performed as part of another procedure, including a spinal decompression procedure, since it may enhance access to regions of the spine requiring decompression.

Also described herein are devices and methods for implanting and anchoring an electrical lead. An electrical lead may be used to help treat chronic pain. The devices and methods described herein may allow precise implantation and anchoring of a lead. Adequate anchoring of implants (such as leads) is critical to prevent migration and eventual failure of these devices.

Also described are methods of treating spinal bone such as facet joints. For example, described herein are methods of resurfacing adjacent facet joints as part of a fusion procedure.

In another variation, method of performing discectomy are also described, which may also be performed as part of a separate procedure, or as part of a decompression procedure.

For example, described herein are methods for placing an inner spinous distractor within a body using a pullwire having a tissue-penetrating distal end and a proximal end. These methods may include: extending a pullwire across an inner spinous ligament between two spinous processes so that the proximal end of the pullwire extends from a first position outside of the body, and the distal end of the pull wire extends from a second position outside of the body; and pulling the distal end of the pullwire to pull a spinous process distractor from the first position into the inner spinous ligament between the two spinous processes.

The method may also include the step of coupling the proximal end of the pullwire to a distal end of the spinous process distractor. For example, the method may include coupling the proximal end of the pullwire to a distal end of a spinous process distractor delivery device. The step of extending the pullwire may include percutaneously passing the pullwire through the body from a first opening in the body at the first position to a second opening in body at the second position.

The method may also include detaching the distal end of the pullwire from the spinous process distractor. The pullwire may then be removed from body; in some variations the pullwire may remain coupled to a portion of the spinous process detractor delivery device, which may be removed with the pullwire.

The method may also include pulling a sizer between the two spinous processes using the pullwire. The sizer may be used to determine the appropriate size spinous process distractor to use.

In some variations the method also includes locking the spinous process distractor in position between the two spinous processes. The method may also include expanding the spinous processes distractor.

The step of extending a pullwire may include inserting a curved, cannulated probe between the spinous processes and passing the pullwire through the cannulated probe to extend from the distal end and out of the second opening out of the body. In some variations, the probe may include an outer cannula and an inner cannula that is configure to be extend from the distal end of the outer cannula in a curved pathway.

Also described herein are methods of placing an inner spinous distractor within a body using a pullwire having a tissue-penetrating distal end and a proximal end, the method comprising: inserting a curved, cannulated probe between two spinous processes so that the tip of the probe extends in a curved pathway through the inner spinous ligament; extending a pullwire through the probe so that a distal end of the pullwire extends out of the body while the proximal end extends from the body proximally; removing the probe while leaving the pullwire in position across the spinous ligament; and pulling the distal end of the pullwire to pull a spinous process distractor between the two spinous processes.

Also described herein are systems for inner spinous distraction, the system comprising: an inner spinous distractor configured to be pulled into position through the inner spinous ligament between two spinous processes and to distract the two spinous processes; a pullwire having a tissue-penetrating distal end and a coupler at the proximal end, the coupler configured to couple to the inner spinous distractor so that the pullwire may be used to pull the inner spinous distractor into position; and a cannulated probe having a curved distal end, the probe configured to position the pullwire between two spinous processes.

In some variations, the system also includes a sizer configured to couple to the proximal end of the pullwire so that it can be pulled between two spinous processes.

The system may also include a distal handle configured to attach to the distal end of the pullwire and to secure the tissue-penetrating distal end of the pullwire.

In some variations the system also includes an inner spinous distractor delivery tool configured to hold the inner spinous distractor for delivery between two spinous processes, wherein the distal end of the delivery tool comprises a coupler for coupling to the proximal end of the pullwire and the proximal end of the inner spinous distractor delivery tool comprises a proximal handle.

The system may also include a lock for securing the inner spinous distractor in position between two spinous processes.

Also described herein are methods of implanting a lead for electrical stimulation adjunct to a target nerve tissue, the method comprising: extending a pullwire adjacent to the target nerve tissue so that the proximal end of the pullwire extends from a first position outside of the body, and the distal end of the pull wire extends from a second position outside of the body; coupling the distal end of the lead to the proximal end of the pullwire; and pulling the distal end of the pullwire to pull an electrical lead from the first position so that the lead is adjacent to the target nerve tissue.

The method may also include the step of anchoring the proximal and distal end of the lead. For example, the step of anchoring the proximal and distal end of the lead may comprise expanding an expandable member, or inflating a balloon.

The method may also include de-coupling the distal end of the lead from the proximal end of the pullwire and withdrawing the pullwire distally from the body.

The step of extending the pullwire may include passing the pullwire over a spinal pedicle. In some variations, the step of extending the pullwire comprises passing the pullwire down the lateral recess between two spinal lamina.

The method may also include confirming the position of the target nerve relative to the path of the guidewire. For example, a nerve localization device (including a plurality of electrodes for stimulating nerves that are immediately near the localization device) may be used, for example, by pulling the neural localization device through the tissue using the pullwire.

Also described herein are electrical leads for pain management that are configured to be pulled into position distally and anchored distally and proximally. For example, such a lead may include: an elongate body having a distal coupling region configured to couple to the proximal end of a pullwire; a first anchoring element at the distal end configured to anchor the lead within the body; a second anchoring element at the proximal end configured to anchor the lead within the body; and a plurality of electrical contacts located between the proximal and distal anchors.

The electrical lead devices may also include a proximally-extending electrical connector configured to connect to an implantable pulse generator for applying energy to the plurality of electrical contacts.

Also described herein are systems for positioning and anchoring an electrical lead relative to a patient's spinal nerves, the system comprising: an electrical lead comprising a distal connector configured to be used to distally pull the lead adjacent to a target spinal nerve tissue; a pullwire having a tissue-penetrating distal end and a coupler at the proximal end, the coupler configured to couple to the distal connector of the electrical lead so that the pullwire may be used to pull the electrical lead into position; and a cannulated probe having a curved distal end, the probe configured to position the pullwire adjacent to the target spinal nerve tissue.

In some variations the system includes a neural localization device having a distal connector configured to couple to the coupler at the proximal end of the pullwire. In some variations the system further comprises a distal handle configured to attach to the distal end of the pullwire and to secure the tissue-penetrating distal end of the pullwire.

Also described herein are methods of fusing a facet joint using a bimanual treatment device. For example, a method of fusing a facet joint using a bimanual treatment device the method may include the steps of: extending a pullwire between two spinous processes so that the proximal end of the pullwire extends from a first position outside of the body, and the distal end of the pull wire extends from a second position outside of the body; coupling the distal end of a facet joint modifying treatment device to the proximal end of the pullwire; pulling the distal end of the pullwire to pull the facet joint modifying treatment device from the first position so that the facet joint modifying treatment device is adjacent to the facet joint; and reciprocating the facet joint modifying treatment device by pulling distally on the pullwire and proximally on the facet joint modifying treatment device.

Many variations of the methods described herein may also include the step of applying a filling material between the facet joint. Filling materials may include cement (e.g., bone cement), graft materials, or the like. The method may also include the step of inserting a support between the facet joint by pulling the cage in distally using the pullwire. For example, the support may comprise a cage, and/or an expandable member.

In some variations the method includes the step of cutting the superior spinous process of the facet.

Any appropriate facet joint modifying treatment device may be used, including a facet joint modifying treatment device having a bone-cutting surface.

Also described herein are methods for precisely placing and/or manipulating devices within the body by first positioning a guidewire through the body from a first location, around a curved pathway, and out of the body through a second location, so that the distal and proximal ends of the guidewire extend from the body, then pulling a device into position using the guidewire. The device to be positioned within the body is coupled to the proximal end of the guidewire, and the device is pulled into the body by pulling on the distal end of the guidewire that extends from the body. The device may be bimanually manipulated by pulling the guidewire distally, and an attachment to the device that extends proximally, allowing control of both the proximal and the distal ends. In this manner devices (and particularly implants such as innerspinous distracters, stimulating leads, and disc slings) may be positioned and/or manipulated within the body. Devices to modify tissue may also be positioned or manipulated so that a target tissue within the body is modified.

Devices and systems configured to be coupled to the proximal end of a pull guidewire (or “pullwire”) are also described. In general, a system for pulling an implant or tissue modification device into position as described herein may include a probe for positioning a guidewire into position, a guidewire/pullwire, a handle for the guidewire/pullwire, and a device having a distal end configured to couple to the pullwire and be pulled into position by the pullwire. The devices or implants may be adapted for use with the pullwire. For example the distal end of the devices/implants may be configured to releaseably secure to the proximal end of the pullwire. Furthermore, the devices may be adapted so that the connection with the guidewire is sufficient to withstand a substantial amount of pulling force that may be applied when positioning or manipulating the device(s).

For example, the some variations of the devices and methods described herein may be used to position and/or manipulate devices involved in the treatment of any of the following conditions: accessing a facet joint for fusion (e.g., posterior lateral gutters), implantation, etc.; spinal fusions, particularly placement of devices or implants within spinal regions such as the facet joint; and the like.

Described below are particular examples, including devices adapted for use with these examples that illustrate methods of performing such treatments and therapies. For example, described herein are methods of performing a spinal fusion. In some embodiments, a spinal decompression procedure may also be performed, since it may enhance success rates of the spinal fusion procedure.

Also described are methods of treating spinal bone such as facet joints. For example, described herein are methods of resurfacing adjacent facet joints as part of a fusion procedure.

Also described herein are methods of fusing a facet joint using a bimanual treatment device. For example, a method of fusing a facet joint using a bimanual treatment device the method may include the steps of: extending a pullwire between two spinous processes so that the proximal end of the pullwire extends from a first position outside of the body, and the distal end of the pull wire extends from a second position outside of the body; coupling the distal end of a facet joint modifying treatment device to the proximal end of the pullwire; pulling the distal end of the pullwire to pull the facet joint modifying treatment device from the first position so that the facet joint modifying treatment device is adjacent to the facet joint; and reciprocating the facet joint modifying treatment device by pulling distally on the pullwire and proximally on the facet joint modifying treatment device.

The method may also include the step of applying a filling material between the facet joint. Filling materials may include cement (e.g., bone cement), graft materials, or the like. The method may also include the step of inserting a support between the facet joint by pulling the cage in distally using the pullwire. For example, the support may comprise a cage, and/or an expandable member.

In some variations the methods include the step of cutting the superior articular process (SAP) and/or inferior articular process (IAP) of the facet.

Any appropriate facet joint modifying treatment device may be used, including a facet joint modifying treatment device having a bone-cutting surface.

Once the joint has been prepared using the device or devices, the device may be removed, and a support structure or material may be added to fuse the joint. The guidewire/pullwire may remain in position, so that it can be used to pull in or apply the material. For example, in some variations the pullwire may be used to position a cage or other mechanical support within the joint. The mechanical support may be coupled to the proximal end of the pullwire directly or indirectly (e.g., via an elongate carrier structure from which it can be released once it is positioned), and pulled into position. In some variations the pullwire may be used to pull a tube or other fluid material delivery device into position in the joint, to apply a filer material such as bone cement, bone graft material, etc. In some variations, the pullwire may be used to pull into position in the joint an expandable or fillable structure that will be implanted in the joint. For example, a mesh or porous “bag” structure may be pulled into position (and decoupled from the pullwire) and filled with appropriate fusing material (e.g., cement, etc.). In some variations a bag or balloon-like structure is pulled into position and filled.

In an alternative variation, the support structure to facilitate fusion may be a cinch or band wrapped around a joint, such as a facet joint. In some embodiments, the support structure may further include a cap or other fixation device to hold the band in place and/or to facilitate and/or promote fusion.

Also described herein are particular examples, including devices adapted for use with these examples that illustrate methods of performing treatments and therapies including spinal fusion surgery, in particular a less invasive posterior column fusion or soft fusion, such as by way of an interspinous fusion or spinous process fixation. Additionally, facet joint fixation may also be performed, either bilaterally or unilaterally. In some embodiments, a spinal decompression procedure may also be performed, since it may enhance success rates of the spinal fusion procedure.

For example, described herein are devices, systems and methods for performing an interspinous fusion unilaterally. The devices and systems described herein are configured to be deployable from a single side of the spine (i.e. unilaterally deployable). In general, the devices and systems described herein include a fixation plate; a coupling screw, sized and configured to couple the fixation plate to a fist lateral side of a spinous process; and a coupling backing, sized and configured to couple to the opposite lateral side of the spinous process. In some embodiments, the devices and systems described herein include a fixation plate sized and configured to couple the fixation plate to a first lateral side of a spinous process; a coupling backing, coupled to the fixation plate and sized and configured to couple to the opposite lateral side of the spinous process, wherein the coupling backing is sized and configured to operate in one of three modes: a non-deployed mode, wherein the coupling backing fits between two adjacent spinous processes, a deployed mode, wherein the coupling backing rotates behind a spinous process, and a compression mode, wherein the coupling backing is compressed against the opposite lateral side of the spinous process.

In general, the methods described herein include the steps of placing a fixation plate adjacent to a first lateral side of a spinous process and deploying a coupling backing such that it couples to the opposite lateral side of the spinous process. In some embodiments, the methods further include the step of placing a coupling screw through the fixation plate from the first lateral side of the spinous process. In some embodiments, the methods described herein include the steps of placing a fixation plate adjacent to a fist lateral side of a spinous process from the fist lateral side of the spinous process; placing a coupling backing in a non-deployed mode between two adjacent spinous processes from the fist lateral side of the spinous process; deploying the coupling backing from the fist lateral side of the spinous process such that it rotates behind the spinous process, adjacent to the opposite lateral side of the spinous process; and compressing the coupling backing against the opposite lateral side of the spinous process.

Also described herein are devices, systems and methods for performing an interspinous fusion unilaterally. As mentioned above, the devices and systems described herein are configured to be deployable from a single side of the spine (i.e. unilaterally deployable). The methods described herein may include the steps of placing a fixation plate adjacent to a first lateral side of a spinous process and deploying a coupling backing such that it couples to the opposite lateral side of the spinous process. In some embodiments, the methods further include the step of placing a coupling screw through the fixation plate from the first lateral side of the spinous process.

In some embodiments, a unilaterally deployable interspinous fusion device includes a fixation plate sized and configured to couple the fixation plate to a fist lateral side of a spinous process; a coupling backing, coupled to the fixation plate and sized and configured to couple to the opposite lateral side of the spinous process, wherein the coupling backing is sized and configured to operate in one of two modes: a non-deployed mode, wherein the coupling backing fits between two adjacent spinous processes, and a deployed mode, wherein the coupling backing rotates behind a spinous process.

In some embodiments, a method of performing an interspinous fusion unilaterally includes the steps of placing a fixation plate adjacent to a fist lateral side of a spinous process from the fist lateral side of the spinous process; placing a coupling backing in a non-deployed mode between two adjacent spinous processes from the fist lateral side of the spinous process; and deploying the coupling backing from the fist lateral side of the spinous process such that it rotates behind the spinous process, adjacent to the opposite lateral side of the spinous process.

In some embodiments, a unilaterally deployable interspinous fusion device includes a fixation plate configured to couple to a first lateral side of a spinous process; a paddle configured to couple to the opposite lateral side of the spinous process; and a post coupled to the fixation plate and configured to receive a paddle extension coupled to the paddle.

In some embodiments, a method of performing an interspinous fusion unilaterally includes the steps of placing a fixation plate adjacent to a fist lateral side of a spinous process from the fist lateral side of the spinous process; placing a paddle from the fist lateral side of the spinous process such that it rotates behind the spinous process, adjacent to the opposite lateral side of the spinous process; and coupling the paddle to the fixation plate.

In some embodiments, a unilaterally deployable interspinous fusion system includes a fixation plate configured to couple to a first lateral side of a spinous process; a fixation screw configured to be implanted into bone on the first lateral side of a spinous process; and an extension configured to couple the fixation plate to the fixation screw.

Also described herein are facet fixation devices and methods. In some embodiments, the fixation device includes a fixation screw configured to be implanted into a facet joint and a facet cap configured to couple to the fixation screw and to couple to a medial aspect and a lateral aspect of a facet joint.

As mentioned, the interspinous fusion devices and systems described herein may be configured to be deployable from a single side of the spine (i.e. unilaterally deployable). The devices and systems described herein may also be configured to also be bilaterally deployable, such that a surgeon (or other user) may deploy the same device either from a unilateral approach or a bilateral approach depending on their preference, the anatomy of the patient, the spinal level to be fused, etc. If a surgeon is fusing multiple spinal levels, the device may be implanted unilaterally for a first level, and bilaterally for a second level, for example. Furthermore, the device may be configured to be deployable via a lateral (e.g. TLIF) approach. The devices and systems described herein may be configured to also be deployable via a midline approach, such that a surgeon (or other user) may deploy the same device either from a lateral approach or a midline approach depending on their preference, the anatomy of the patient, the spinal level to be fused, etc.

In some variations of the methods described herein, the methods include the steps of placing a fixation plate adjacent to a first lateral side of a spinous process and deploying a coupling backing such that it couples to the opposite lateral side of the spinous process. In some embodiments, the methods further include the step of placing a coupling screw through the fixation plate from the first lateral side of the spinous process.

In some embodiments, a unilaterally deployable interspinous fusion device includes a fixation plate sized and configured to couple the fixation plate to a fist lateral side of a spinous process; a coupling backing, coupled to the fixation plate and sized and configured to couple to the opposite lateral side of the spinous process, wherein the coupling backing is sized and configured to operate in one of two modes: a non-deployed mode, wherein the coupling backing fits between two adjacent spinous processes, and a deployed mode, wherein the coupling backing rotates behind a spinous process.

In some embodiments, a unilaterally deployable interspinous fusion device includes a first fixation plate configured to be deployed from a first lateral side of a spinous process and to couple to the opposite lateral side of the spinous process; a rod coupled to the first fixation plate such that the first fixation plate is rotatable with respect to the rod; and a second fixation plate configured to be deployed from the first lateral side of a spinous process and to couple to the first lateral side of a spinous process, wherein the second fixation plate is configured to couple to the rod such that the first and second fixation plates are compressed about the spinous process.

In some embodiments, the device further includes a barrel. In some embodiments, the barrel is configured to receive graft material. In some embodiments, the barrel is configured to lock the first fixation plate in position with respect to the rod.

Some of the methods described herein may include the steps of placing a first fixation plate coupled to a rod from the fist lateral side of the spinous process; rotating the plate with respect to the rod such that the plate couples to the opposite lateral side of the spinous process; placing a second fixation plate adjacent to a fist lateral side of a spinous process from the fist lateral side of the spinous process; and coupling the second the fixation plate to the rod.

As just mentioned, some of the devices, systems and methods for performing an interspinous fusion described herein are adapted for use unilaterally, but may also be configured to also be bilaterally deployable, such that a surgeon (or other user) may deploy the same device either from a unilateral approach or a bilateral approach depending on their preference, the anatomy of the patient, the spinal level to be fused, etc. If a surgeon is fusing multiple spinal levels, the device may be implanted unilaterally for a first level, and bilaterally for a second level, for example. Furthermore, the device may be configured to be deployable via a lateral (e.g. TLIF) approach. The devices and systems described herein may be configured to also be deployable via a midline approach, such that a surgeon (or other user) may deploy the same device either from a lateral approach or a midline approach depending on their preference, the anatomy of the patient, the spinal level to be fused, etc.

In some embodiments, a unilaterally deployable interspinous fusion device includes a fixation plate configured to couple to a first lateral side of a spinous process; a paddle configured to couple to the opposite lateral side of the spinous process; and a spacer configured to receive a paddle extension coupled to the paddle.

In some embodiments, a method of performing an interspinous fusion unilaterally includes the steps of placing a paddle, from a fist lateral side of the spinous process, behind a spinous process adjacent to the opposite lateral side of the spinous process; placing a fixation plate adjacent to the fist lateral side of a spinous process from the fist lateral side of the spinous process; and coupling the paddle to the fixation plate.

In some embodiments, the method further includes the step of placing a spacer between two adjacent spinous processes. In some embodiments, the method further includes the step of separating a first paddle from a second paddle, wherein the two paddles are adjacent to the opposite lateral side of the spinous processes. In some embodiments, the method further includes the step of placing a spacer between two adjacent spinous processes and maintaining the distance between the two paddles. In some embodiments, the method further includes the step of clamping the paddle and the fixation plate against the lateral sides of the spinous processes.

In some variations, the devices for interspinous fusion may be configured to be deployable via a lateral (e.g. TLIF) approach. The devices and systems described herein may be configured to also be deployable via a midline approach, such that a surgeon (or other user) may deploy the same device either from a lateral approach or a midline approach depending on their preference, the anatomy of the patient, the spinal level to be fused, etc.

In some embodiments, a unilaterally deployable interspinous fusion device includes a fixation plate configured to couple to a first lateral side of a spinous process; a paddle configured to couple to the opposite lateral side of the spinous process; and a spacer configured to receive a paddle extension coupled to the paddle.

In some embodiments, a method of performing an interspinous fusion unilaterally includes the steps of placing a paddle, from a fist lateral side of the spinous process, behind a spinous process adjacent to the opposite lateral side of the spinous process; placing a fixation plate adjacent to the fist lateral side of a spinous process from the fist lateral side of the spinous process; and coupling the paddle to the fixation plate.

In some embodiments, the method further includes the step of placing a spacer between two adjacent spinous processes. In some embodiments, the method further includes the step of separating a first paddle from a second paddle, wherein the two paddles are adjacent to the opposite lateral side of the spinous processes. In some embodiments, the method further includes the step of placing a spacer between two adjacent spinous processes and maintaining the distance between the two paddles. In some embodiments, the method further includes the step of clamping the paddle and the fixation plate against the lateral sides of the spinous processes.

Also described herein are devices, systems and methods for deploying a graft implant unilaterally. In some embodiments, the graft implant includes a graft body. In some embodiments, the graft implant further includes a coupling member on the graft body. In some embodiments, the graft implant further includes a first material with a first fusion capability and a second material with a second distinct fusion capability. In some embodiments, the graft implant is configured to receive bone material.

In some embodiments, the graft implant further includes a locking mechanism configured to hold the graft in a portion of a spine. In some embodiments, the locking mechanism comprises a pedicle screw and a connecting rod. In some embodiments, the graft body is configured to receive the connecting rod. In some embodiments, the graft body includes a plurality of graft sections. In some embodiments, the graft body includes a flexible sack.

The methods for interspinous fusion described herein may also include the steps of placing a fixation plate adjacent to a first lateral side of a spinous process and deploying a coupling backing such that it couples to the opposite lateral side of the spinous process. In some embodiments, the methods further include the step of placing a coupling screw through the fixation plate from the first lateral side of the spinous process.

In some embodiments, a unilaterally deployable interspinous fusion device includes a fixation plate sized and configured to couple the fixation plate to a fist lateral side of a spinous process; a coupling backing, coupled to the fixation plate and sized and configured to couple to the opposite lateral side of the spinous process, wherein the coupling backing is sized and configured to operate in one of two modes: a non-deployed mode, wherein the coupling backing fits between two adjacent spinous processes, and a deployed mode, wherein the coupling backing rotates behind a spinous process.

In some embodiments, a unilaterally deployable interspinous fusion device includes a first fixation plate configured to be deployed from a first lateral side of a spinous process and to couple to the opposite lateral side of the spinous process; a rod coupled to the first fixation plate such that the first fixation plate is rotatable with respect to the rod; and a second fixation plate configured to be deployed from the first lateral side of a spinous process and to couple to the first lateral side of a spinous process, wherein the second fixation plate is configured to couple to the rod such that the first and second fixation plates are compressed about the spinous process.

In some embodiments, the device further includes a barrel. In some embodiments, the barrel is configured to receive graft material. In some embodiments, the barrel is configured to lock the first fixation plate in position with respect to the rod. In some embodiments, the device further includes a handle. In some embodiments, the handle includes an internal handle portion that couples to a joint between the first fixation plate and the rod and an external handle portion that couples to the rod. In some embodiments, the internal handle portion functions to lock the fixation plate with respect to the rod.

Some variations of the methods for interspinous fusion described herein include the steps of placing a first fixation plate coupled to a rod from the fist lateral side of the spinous process; rotating the plate with respect to the rod such that the plate couples to the opposite lateral side of the spinous process; placing a second fixation plate adjacent to a fist lateral side of a spinous process from the fist lateral side of the spinous process; and coupling the second the fixation plate to the rod. In some embodiments, the method further includes the step of coupling a handle to the rod. In some embodiments, after the rotating step, the method further includes the step of locking the plate with respect to the rod with the handle.

Also described herein are unilaterally deployable interspinous fusion devices. In general, in some embodiments, a fusion device may include a fixation plate sized and configured to couple the fixation plate to a fist lateral side of a spinous process; a coupling backing, coupled to the fixation plate and sized and configured to couple to the opposite lateral side of the spinous process, wherein the coupling backing is sized and configured to operate in one of two modes: a non-deployed mode, wherein the coupling backing fits between two adjacent spinous processes, and a deployed mode, wherein the coupling backing rotates behind a spinous process.

In some embodiments, a method of performing an interspinous fusion unilaterally may include the steps of placing a fixation plate adjacent to a fist lateral side of a spinous process from the fist lateral side of the spinous process, placing a coupling backing in a non-deployed mode between two adjacent spinous processes from the fist lateral side of the spinous process, and deploying the coupling backing from the fist lateral side of the spinous process such that it rotates behind the spinous process, adjacent to the opposite lateral side of the spinous process.

In some embodiments, a unilaterally deployable interspinous fusion device may include a fixation plate configured to couple to a first lateral side of a spinous process, a paddle configured to couple to the opposite lateral side of the spinous process, and a post coupled to the fixation plate and configured to receive a paddle extension coupled to the paddle.

In some embodiments, a method of performing an interspinous fusion unilaterally may include the steps of placing a fixation plate adjacent to a fist lateral side of a spinous process from the fist lateral side of the spinous process, placing a paddle from the fist lateral side of the spinous process such that it rotates behind the spinous process, adjacent to the opposite lateral side of the spinous process, and coupling the paddle to the fixation plate.

In some embodiments, a unilaterally deployable interspinous fusion device may include a fixation plate configured to couple to a first lateral side of a spinous process, a paddle configured to couple to the opposite lateral side of the spinous process; and a spacer configured to receive a paddle extension coupled to the paddle.

In some embodiments, a method of performing an interspinous fusion unilaterally may include the steps of placing a paddle, from a fist lateral side of the spinous process, behind a spinous process adjacent to the opposite lateral side of the spinous process; placing a fixation plate adjacent to the fist lateral side of a spinous process from the fist lateral side of the spinous process; and coupling the paddle to the fixation plate. The method may further include the step of placing a spacer between two adjacent spinous processes. In some embodiments, the method may further include the step of separating a first paddle from a second paddle, wherein the two paddles are adjacent to the opposite lateral side of the spinous processes. The method may further include the step of placing a spacer between two adjacent spinous processes and maintaining the distance between the two paddles. The method may further include the step of clamping the paddle and the fixation plate against the lateral sides of the spinous processes.

In some embodiments described herein, an interspinous fusion system may include a first fixation plate configured to couple to a first lateral side of a spinous process, a rod extending from the first fixation plate at a joint such that the rod is pivotable with respect to the first fixation plate, and a second fixation plate configured to couple to a second lateral side of a spinous process opposite from the first fixation plate, wherein the second fixation plate is configured to couple to the rod such that the first and second fixation plates are compressed about the spinous process.

In some embodiments, the first fixation plate further includes a slot sized and configured to receive a portion of the rod when the rod is pivoted with respect to the fixation plate such that the rod is not perpendicular to the plate. In some embodiments, the second fixation plate is configured to be deployed from a first lateral side of a spinous process and to couple to the first lateral side of a spinous process and wherein the first fixation plate is configured to be deployed from the first lateral side of a spinous process and to couple to the second opposite lateral side of the spinous process. In some embodiments, at least one of the first and the second fixation plate further includes a coupling member disposed on the inside face of the fixation plate, wherein the coupling member of the inside face are configured to couple to the first or second lateral side of the spinous process. In some embodiments, the coupling member includes at least one protrusion sized and configured to dig into or catch onto bone thereby aiding in the coupling of the fixation plate to the spinous process.

In some embodiments, the second fixation plate further includes a locking member configured to lock the second fixation plate onto the rod. In some embodiments, the rod includes a non-circular cross section. In some embodiments, the rod is threaded along a portion of its length. In some embodiments, the rod includes both internal and external threads along a portion of its length. In some embodiments, the joint is configured such that the rod is slidable with respect to the first fixation plate. In some embodiments, the rod is pivotable with respect to the first fixation plate such that the rod may be positioned into an introduction position such that the angle between the fixation plate and the rod is less than 90 degrees, wherein the introduction position allows the first fixation plate to be deployed from the first lateral side of a spinous process and to couple to the second opposite lateral side of the spinous process. In some embodiments, the angle between the fixation plate and the rod in the introduction position is about 0 degrees. In some embodiments, the rod is pivotable with respect to the first fixation plate such that the rod may be positioned into a locking position having an angle between the fixation plate and the rod about equal to 90 degrees.

In some embodiments, the system further includes a handle configured to couple to the rod. In some embodiments, the handle includes an external handle portion and an internal handle portion disposed within the external handle portion, and wherein the external handle portion couples to external threads of the rod and the internal handle portion couples to internal threads of the rod. In some embodiments, a distal end of the internal handle portion further includes a locking portion, sized and configured to couple to the joint between the plate and the rod and to lock the plate position with respect to the rod. In some embodiments, the external handle portion is configured to couple to a non-circular cross section of the rod.

In some embodiments, the system further includes a barrel sized and configured to be positioned over the rod and between two adjacent spinous processes. In some embodiments, the barrel is sized and configured to be positioned over the rod such that the barrel will not rotate about the rod. In some embodiments, the barrel is configured to receive graft material. In some embodiments, the barrel includes a window sized and configured to allow graft material, disposed within the barrel, to contact an adjacent spinous process. In some embodiments, the barrel is further sized and configured to distract two adjacent spinous processes. In some embodiments, the barrel is configured to lock the first fixation plate in position with respect to the rod. In some embodiments, the barrel includes a tab that extends from the end of the barrel and is sized and configured to couple to a slot in the first fixation plate such that the barrel locks the first fixation plate in position with respect to the rod. In some embodiments, the barrel further includes a rod locking mechanism configured to lock the barrel onto the rod.

In some embodiments, at least one of the first and second fixation plates further include a tapered leading edge configured to dissect tissue from a lateral side of the spinous process.

Also described herein is a method of performing an interspinous fusion unilaterally. In some embodiments, the method may include the steps of placing a first fixation plate, having a rod extending from the fixation plate, between two adjacent spinous processes from a fist lateral side of the spinous processes; pivoting the rod with respect to the first fixation plate such that the plate abuts the second, opposite, lateral side of at least one of the spinous processes; placing a second fixation plate such that it abuts the fist lateral side of at least one of the spinous processes from the fist lateral side of the spinous processes; and coupling the second the fixation plate to the rod.

In some embodiments, pivoting the rod further includes sliding the rod with respect to the first fixation plate. In some embodiments, coupling the second the fixation plate to the rod further includes coupling the second the fixation plate to the rod such that the first and second fixation plates are compressed about the spinous process. In some embodiments, coupling the second the fixation plate to the rod further includes locking the second fixation plate onto the rod. In some embodiments, placing the first fixation plate further includes positioning the rod into an introduction position with respect to the first fixation plate such that the angle between the fixation plate and the rod is less than 90 degrees. In some embodiments, the angle between the fixation plate and the rod is about 0 degrees. In some embodiments, placing the first fixation plate further includes positioning the rod into a locking position with respect to the first fixation plate such that the angle between the fixation plate and the rod is about equal to 90 degrees.

In some embodiments, the method further includes the step of coupling a handle to the rod. In some embodiments, coupling a handle to the rod further includes coupling an external handle portion to external threads of the rod and coupling an internal handle portion to internal threads of the rod. In some embodiments, coupling a handle to the rod further includes locking the rod in position with respect to the first fixation plate with a locking portion of an internal handle portion.

In some embodiments, the method further includes the step of positioning a barrel over the rod and between the two adjacent spinous processes. In some embodiments, the method further includes the step of placing graft material within the barrel. In some embodiments, positioning the barrel over the rod further includes distracting two adjacent spinous processes with the barrel. In some embodiments, positioning the barrel over the rod further includes locking the first fixation plate in position with respect to the rod with the barrel. In some embodiments, the method further includes the step of dissecting tissue from the first or second lateral side of a spinous process with a tapered edge of the first or second fixation plate.

### BRIEF DESCRIPTION OF THE DRAWINGS

FIG. 1 shows a median sagittal section of two lumbar vertebra and their ligaments.

FIG. 2 is a transverse section through the spine, showing the lamina and the superior and transverse processes.

FIGS. 3A-3F illustrate one variation of a system with tools for bimanual treatment of tissue; this variation includes: two variations of a guidewire or pullwire positioning probe tool (3A and 3B), a flexible neural localization tool (3C), a tissue modification tool (3D), a removable guidewire handle (3E), and a guidewire (3F).

FIG. 4A illustrates a facet joint 1005 including the superior and inferior surfaces.

FIG. 4B shows another portion of a spine including a facet joint 1011 that may be fused as described herein.

FIGS. 5A-5E illustrate variations of joint treatment devices. In FIG. 5A, the treatment device includes a front and a back articulating surface that can be drawn across the joint surfaces to roughen them; FIGS. 5B-5D show different cross-sections through joint treatment devices, and FIG. 5E illustrates another variation of a joint treatment device. Any of these joint treatment devices may be facet joint treatment devices.

FIG. 6A illustrates a cross-section through one variation of a facet-joint modifying device that includes two bone-sawing elements.

FIG. 6B illustrates a cross-section through one portion of the device having a breakable spacer.

FIG. 6C shows a top view of one variation of a facet-joint modifying device configured to perform a facetectomy.

FIGS. 7A, 7B and 8A-8H show an embodiment of a system and method for bone plug delivery.

FIG. 9 shows an embodiment of a system and method for bone plug delivery.

FIGS. 10A-10B and 11A-11B show an embodiment of a system and method for support delivery.

FIGS. 12 and 13A-13B show an embodiment of a system and method for delivering a band around a joint.

FIGS. 14-19 show an embodiment of a system and method for delivering a band around a joint.

FIGS. 20 and 21A-21C show embodiments of a band as described herein.

FIG. 22 shows a method of preparing a bone surface for a band.

FIGS. 23A-23B and 24A-24B show alternative embodiments of a band as described herein.

FIGS. 25-29 show three dimensional views of an embodiment of a strap and cap device as described herein. FIG. 27 shows an implanted strap and cap device in cross section.

FIGS. 30A-30E show a method of implanting an embodiment of a strap and cap device as described herein.

FIGS. 31-33B show an exemplary embodiment of a facet fixation system.

FIGS. 34-36D show an exemplary embodiment of a facet fixation system.

FIGS. 37A to 38E show an exemplary embodiment of a facet fixation system.

FIGS. 39-43B show an embodiment of a system and method for delivering a Spinous process fixation system.

FIGS. 44A and 44B show an exemplary embodiment of an interspinous fusion device, specifically one that may be deployable unilaterally. In this example, the fixation device includes a contralateral trans-laminar facet screw.

FIGS. 45A-49B show various exemplary embodiments of an interspinous fusion device, specifically one that may be deployable unilaterally. In these examples, the fixation devices include deployable coupling backings.

FIGS. 50A-51 show an exemplary embodiment of an interspinous fusion device, specifically one that may be deployable unilaterally. In this example, the fixation device may include, for example, an ipsilateral trans-facet interpedicular screw, a trans-facet screw, or a pedicle screw.

FIGS. 52A-54 show an exemplary embodiment of an interspinous fusion device, specifically one that may be deployable unilaterally. In this example, the fixation device may include, for example, an ipsilateral trans-facet interpedicular screw, a pedicle screw, or a trans-facet screw in addition to a contralateral trans-laminar facet screw. FIGS. 52A-54 also show an exemplary method for performing an interspinous fusion unilaterally. In this example, the method may also include the optional step of performing a decompression, as shown in FIG. 30B.

FIGS. 55-57B show an exemplary embodiment of a fusion device, specifically one that may be deployable unilaterally. In this example, the fixation device may be sized and configured to be used in a method for performing an interlaminer fusion unilaterally.

FIGS. 58 to 59B, show an exemplary embodiment of an interspinous fusion device, specifically one that may be deployable unilaterally. In this example, the fixation device may include, for example, an ipsilateral trans-facet interpedicular screw, a pedicle screw, or a trans-facet screw.

FIGS. 60A and 60B show various exemplary embodiments of an interspinous fusion device, specifically one that may be deployable unilaterally. In these examples, the fixation devices include deployable coupling backings.

FIGS. 61-62B show an exemplary embodiment of an interspinous fusion device, specifically one that may be deployable unilaterally.

FIGS. 63A-63C show an exemplary embodiment of an interspinous fusion device, specifically one that may be deployable unilaterally.

FIGS. 64-72 show an exemplary embodiment of an interspinous fusion device, specifically one that includes at least one paddle and that may be deployable unilaterally.

FIGS. 73A-73E show an exemplary embodiment of an interspinous fusion device, specifically one that includes at least one paddle and that may be deployable unilaterally.

FIGS. 74A-76B show an exemplary embodiment of an interspinous fusion device, specifically one that may be deployable unilaterally. In this example, the fixation device includes a slideable fixation plate.

FIG. 77 shows an exemplary embodiment of an interspinous fusion device, specifically one that may be deployable unilaterally. In this example, the fixation device may include, for example, an ipsilateral trans-facet interpedicular screw, a pedicle screw, or a trans-facet screw.

FIGS. 78A-79 show an exemplary embodiment of an interspinous fusion device, specifically one that may be deployable unilaterally. In this example, the fixation device may include, for example, a pedicle screw and a rod.

FIGS. 80A and 80B show a front view and a back views, respectively, of an exemplary embodiment of an interspinous fusion system, specifically one that may be deployable unilaterally.

FIGS. 81A-83E show an exemplary method for performing an interspinous fusion unilaterally using the device of FIGS. 80A and 80B, for example.

FIGS. 84A-86 show an exemplary embodiment of a handle used for performing an interspinous fusion unilaterally using the system of FIGS. 80A and 80B, for example.

FIG. 87 shows an alternative exemplary embodiment of an interspinous fusion device, specifically one that may be deployable unilaterally.

FIGS. 88-90B show an exemplary embodiment of an interspinous fusion device, specifically one that may be deployable unilaterally.

FIGS. 91A-91D and 92 show an exemplary embodiment of an interspinous fusion fixation plate (and method of deploying the plate), specifically one that may be deployable unilaterally.

FIGS. 93A and 93B show an exemplary embodiment of an interspinous fusion fixation device, specifically one that comprises the fixation plate of FIGS. 91A-92.

FIGS. 94A-94E show an exemplary embodiment of an interspinous fusion device, specifically one that may be deployable unilaterally.

FIGS. 95A-96D show exemplary embodiments of interspinous fusion devices, specifically ones that may be deployable unilaterally and include a first and second rotatable fixation plate.

FIGS. 97A-99 show exemplary embodiments of interspinous fusion devices, specifically ones that may be deployable unilaterally and include a first and second fixation bracket.

FIGS. 100A-100C show an exemplary embodiment of an interspinous fusion device, specifically one that may be deployable unilaterally.

FIG. 101 shows an exemplary embodiment of an interspinous fusion device, specifically one that may be deployable unilaterally.

FIG. 102 shows a top view of an exemplary embodiment of an interspinous fusion device, specifically one that includes at least one paddle and that may be adjustable in at least the cephalad/caudad direction.

FIG. 103 shows a top view of an alternative exemplary embodiment of an interspinous fusion device, specifically one that includes at least one paddle and that may be adjustable in at least the cephalad/caudad direction.

FIG. 104 shows an exemplary embodiment of an interspinous fusion device, specifically one that may be deployable unilaterally and that may be adjustable in at least the cephalad/caudad direction.

FIGS. 105A and 105B show an exemplary embodiment of an interspinous fusion device that may be adjustable in at least the cephalad/caudad direction.

FIGS. 106A-106C show an alternative exemplary embodiment of an interspinous fusion device that may be adjustable in at least the cephalad/caudad direction.

FIGS. 107A-107D show an exemplary embodiment of an interspinous fusion device having an adjustable spacer.

FIG. 108 shows an exemplary embodiment of an interspinous fusion device, specifically one that may be deployable unilaterally.

FIG. 109 shows an exemplary embodiment of an interspinous fusion device, specifically one that includes post and that may be deployable unilaterally.

FIG. 110 shows an exemplary embodiment of a tool that may be used to prepare a facet joint and that may be deployable unilaterally.

FIGS. 111-116 show exemplary embodiments of a graft implant.

FIGS. 117A and 117B show a spine having undergone a Facetectomy and exemplary embodiments of an implanted graft implant.

FIGS. 118A and 118B show an embodiment of a graft implant configured to receive a rod.

FIGS. 119A and 119B show an embodiment of a modular graft implant configured to receive a rod.

FIGS. 120A-120C show an embodiment of a modular graft implant configured to couple to a rod.

FIGS. 121A and 121B show an embodiment of a flexible graft implant configured to receive a rod.

FIG. 122 shows an embodiment of a graft implant, a rod, and a locking mechanism.

FIGS. 123-124B show exemplary embodiments of a graft implant with an integrated rod.

FIGS. 125A-125C shows an exemplary embodiment of a modular graft implant with an integrated rod.

FIGS. 126A-127B illustrate methods and devices for introducing spinal fixation elements into a patient's spinal column.

FIG. 128A shows a cross-section though a portion of the spine, indicating the more dense cortical bone regions.

FIGS. 128B and 128C illustrate one variation of a PLIF-type procedure that is made more effective using the pullwire techniques described herein.

FIG. 129 shows an axial view of a vertebra and intervertebral disc. The disc is shown having two anterior quadrants and two posterior quadrants.

FIGS. 130-157B illustrate exemplary methods, devices, and systems for removing tissue and delivering an implant. In particular, the methods, devices, and systems illustrated are for removing spinal disc tissue (e.g. performing a discectomy) and delivering an interbody implant typically as part of a Spinal Fusion Procedure and/or Disc Replacement Procedure. For example, FIGS. 140A-143B and 156A-157B show exemplary devices, such as tissue modification devices.

### DETAILED DESCRIPTION OF THE INVENTION

The systems, methods and devices described herein are generally applicable and adapted for use in treating the spine. Although numerous variations of systems, methods and types of devices for treating the spine are described may be used in one or more spinal procedure without requiring or reference to other aspects of the methods, systems and devices described herein, it is intended that many of the features illustrated may be adapted for use, and/or combined with, one or more features, steps or entire devices or methods described.

For example, some of the devices, systems and methods described herein are adapted for bimanual use, while other devices, systems and methods are adapted for unimanual use. Simialrly, some of the devices, methods and systems described herein are adapted for unilateral insertion/removal, while other devices, methods and systems are adapted for bilateral operation.

Thus, in some variations of the devices, systems and methods for treating tissue described herein, the system and methods is adapted to first place a guidewire (or “pullwire”) in position within the body, and then using the guidewire to position, anchor and/or treat the tissue. For example, the pullwire may be used to pull in interbody cages, fixation screws, autografts, an allografts, or a xenografts, decompression devices, tissue modification devices, or any other suitable component or treatment of a spinal fusion procedure. In general, these methods and systems are “bimanual” procedures, in which the implant or tissue modification device is controlled within the body from two separate locations outside of the body, and by manipulating the implant/device from both the distal and proximal ends.

These systems and methods may be particularly useful for percutaneous treatments of one or more body region. However, it should be understood than any of the devices, methods and systems described herein may be used as part of an “open” surgical procedure in which access to a body region is created through an opening in the tissue (e.g., by removal of tissue). Any of the systems and devices described may be performed as part of a procedure that is at least partially open. Partially percutaneous procedures may also be performed using these devices, systems and methods.

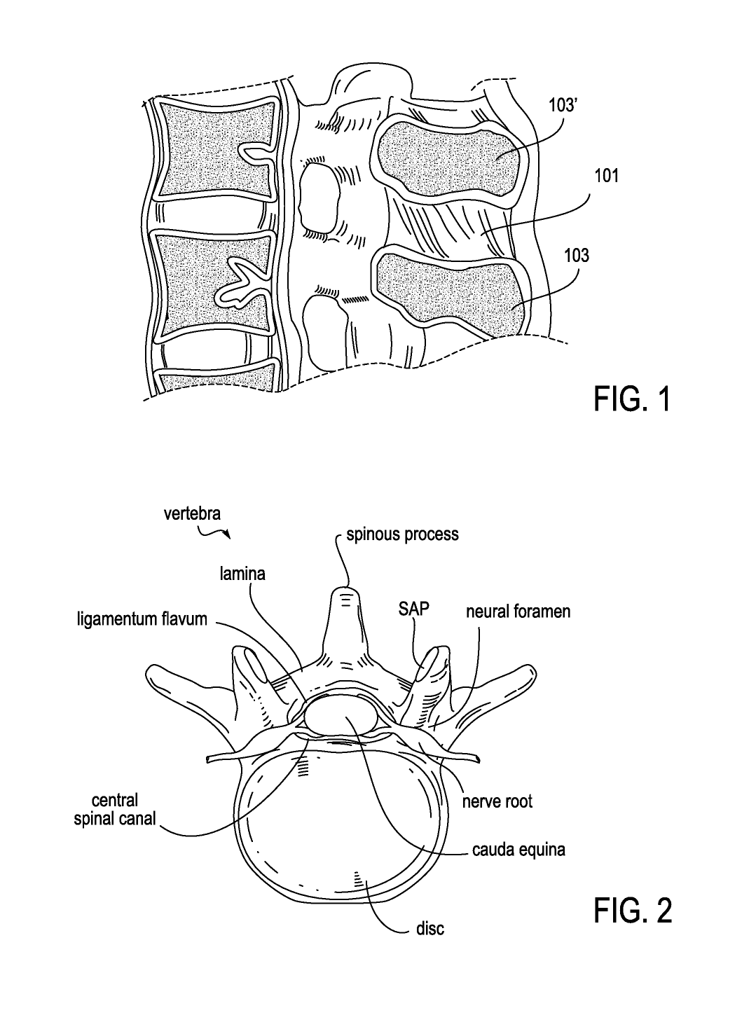

FIGS. 1 and 2 illustrate sections through a normal spine region, including the inter spinous process region. FIG. 1 shows a median sagital section of two lumbar vertebra and their ligaments. In FIG. 1, the section through a region of spine illustrates the inter-spinal ligament 101 connected between two spinous processes 103, 103′. FIG. 2 illustrates a transverse section through the spine, showing the lamina and the superior and transverse processes.

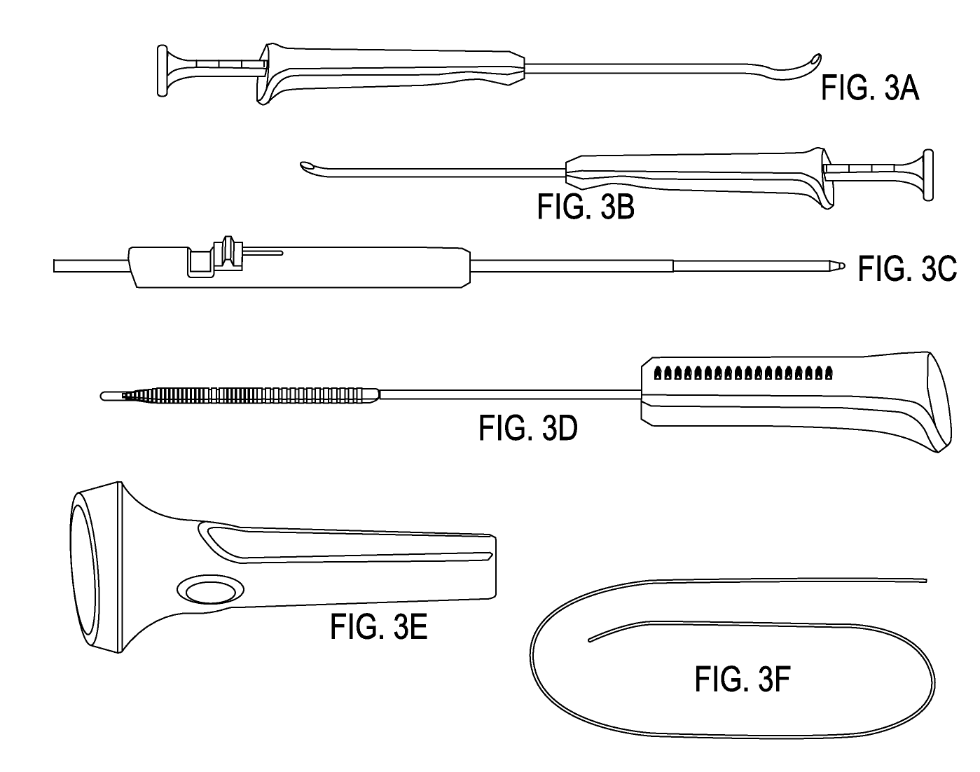

FIGS. 3A-3F illustrate components a system that may be used to treat tissue as described herein. The components illustrated in FIGS. 3A-3F include: two variations of probes (3A and 3B) that may be used to position a guidewire (or pullwire) in the tissue, a neural localization device (FIG. 3C) that is configured to be coupled to the proximal end of a guidewire, a tissue modification device (FIG. 3D) that is configured to scrape or cut tissue and be coupled distally to the proximal end of a guidewire, as well as a guidewire (FIG. 3F) and a handle that may be secured to the distal end of the guidewire (FIG. 3E) allowing manipulation of the distal end of the guidewire/pullwire.

In particular, the guidewire, guidewire handle and placement probes (FIGS. 3A, 3B, 3E and 3F) may be used with one or more additional components to treat a patient, as illustrated in the examples below. In general, these devices may be used to place the guidewire in position within the body so that the (often sharp) distal end of the guidewire extends from the body, and the distal end of the guidewire (which may be adapted to couple to another device so that force can be applied by pulling on the guidewire) extends from a second location in the body.

As mentioned, the proximal end of the guidewire may be adapted to couple to another device or devices. Examples of guidewires that may be used are described, for example, in co-pending applications Ser. No. 11/468,247, titled “TISSUE ACCESS GUIDEWIRE SYSTEM AND METHOD” filed Aug. 29, 2006, and Ser. No. 12/127,535, titled “GUIDEWIRE EXCHANGE SYSTEMS TO TREAT SPINAL STENOSIS” filed May 27, 2008. The distal end of the implant or device to be positioned and/or manipulated may also be adapted to couple to the guidewire as described.

Described herein is a guidewire- or pullwire-based system for distracting a bone or region including bone. These methods may be used to distract bone to treat a compression fracture (e.g., a spinal compression fracture) or to separate bones or bony regions to allow access for further treatment. For example, an access system such as a pullwire-based system can be used to deliver a percutaneous distraction system for distracting the inner spinous process and delivering an inner spinous process distraction device (IPD). Alternatively, a pullwire-based system may be used to position implants, grafts, or other material to facilitate a fusion. Thus, in some variations, described herein are percutaneous inner spinous distraction access and decompression systems, devices and methods of using them.

As described in greater detail below, an inner spinous process distraction device (IPD) may be inserted using the pullwire system. This method of distracting the spinous processes may be used in conjunction with (or as part of) a procedure for decompressing the spine including delivering a transforaminal guide through the foramen. With the IPD holding a foramina open, a decompression procedure can be performed.

Described herein are methods for performing spinal fusion by treating or preparing one or more joints. For example, the devices, systems and methods described herein may be used to resurface a joint, including resurfacing of cartilage and preparation for fusion of the joint. For example, a probe may be used to insert a guidewire between the sides or walls of a joint (e.g., a bone joint). As before, the wire may extend from a first (proximal) site through the body around and/or through the joint, and out of a second (distal) site of the body, allowing bimanual control. A tissue modification device that is configured to resurface the joint may then be coupled to the distal end of the guidewire/pullwire and pulled into position within the joint and used to resurface the joint.

For example, in one variation, the methods and systems described herein include facet joint fusion methods and systems. A facet joint may be fused by first accessing the joint, then preparing the joint and particularly the joint surface(s) (e.g., by roughening or abrading). The joint may then be fixed using a support (e.g., a cage, etc.) or a settable material (bone cement) or graft material. In some variations the fixation step (which may be optional) may include pulling an expandable or fillable material into position and expanding and/or filling it with material.

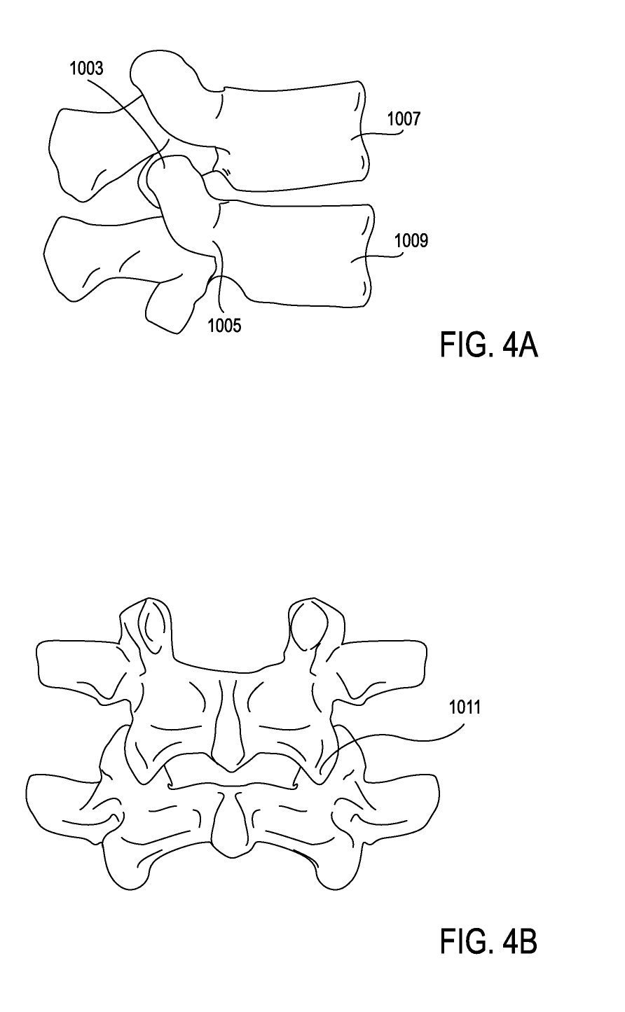

In one variation of a method for fusing a facet joint, a cannulated probe for guiding a guidewire/pullwire is first inserted in and/or around the joint. FIG. 4A illustrates a facet joint 1005 including the superior and inferior surfaces between the lower 1009 and upper 1007 vertebra. A guidewire/pullwire may be threaded through the facet joint as indicated by the line 1003. In some variations, the pathway through the facet joint passes over the top of the superior articulating process (SAP). In some variations, the pathway through the facet joint passes under the SAP giving access to the tip of the SAP. Placement of the guidewire around or through the facet joint may be aided by distraction of the spinous process, as described above. Thus, in some variations, the spinous process may be distracted before performing the procedure. FIG. 4B shows another portion of a spine including a facet joint 1011 that may be fused as described herein.

Once the probe has been used to position the guidewire, it may be removed. As illustrated above, the probe may include one or a plurality of (concentric) cannula including cannulas having different curvatures so that the guidewire may be directed around the joint and pointed toward the appropriate exit site. The guidewire or pullwire may then be pushed through the cannula and out of the patient. A distal handle may then be attached to the distal end of the guidewire to aid in manipulating the guidewire/pullwire from the distal end.

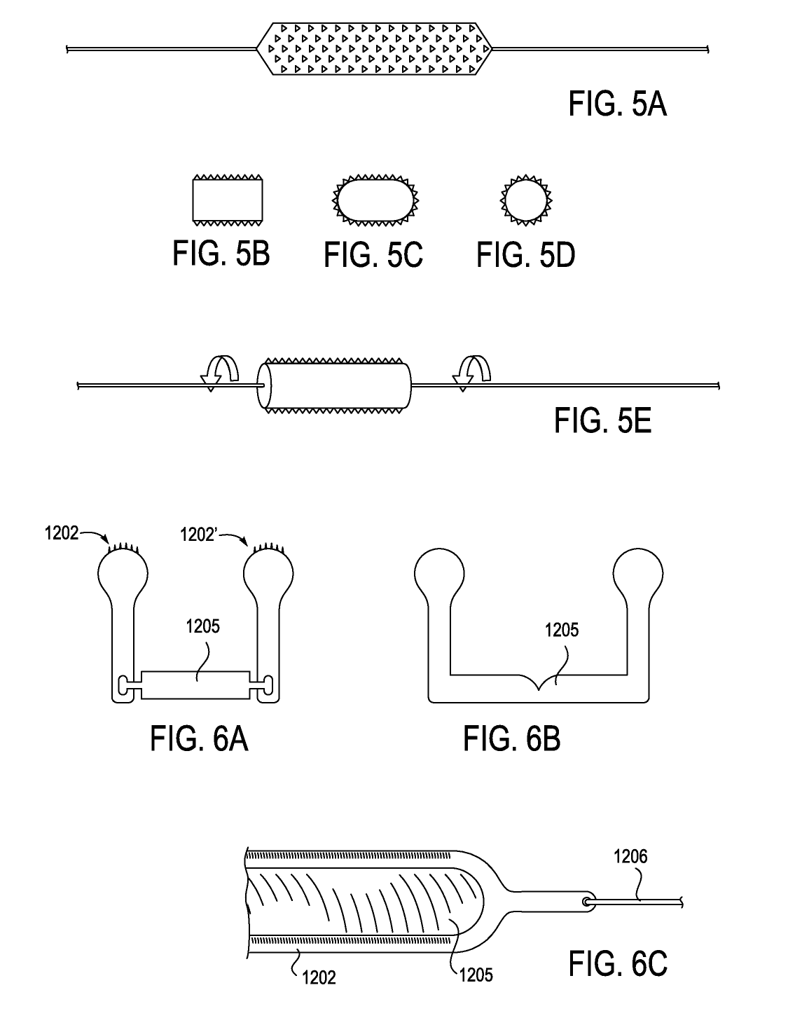

Next, a treatment device may be pulled into position in the joint by coupling the distal end (or end region) of the joint treatment device to the proximal end of the guidewire/pullwire. In some variations the treatment device includes one or more surfaces that are configured to abrade, scratch or otherwise prepare the surface for the fusion. For example, FIGS. 5A-5E illustrate variations of a treatment device. In FIG. 5A, the treatment device includes a front and a back articulating surface that can be drawn across the joint surfaces to roughen them. In this example, the distal end of the device includes an attachment/connector site for the guidewire. The proximal end also includes an elongate member and may have a proximal handle. In some variations the roughening surface is expandable, so that it may be pulled into the joint in a collapsed or condensed form (protecting non-target tissue), and once in the joint it can be expanded to the treatment form. For example, the device may be inflatable; inflation may expand the device so that the contact surface(s) can push against the joint surface(s). Once in position, the device can be moved bimanually within the joint to scrape or otherwise modify the joint surfaces, by pulling distally and proximally (e.g., back and forth).

FIGS. 5B-5E illustrate alternative cross-sections for the joint treatment devices described. For example, in FIG. 5B, the device is substantially flat, having an upper and lower surface. As mentioned, this device may be inflatable/expandable to increase (or decrease) the spacing between the upper and lower surfaces, or to “stiffen” the implant once it is expanded. FIG. 5C shows a device having an oval cross-section, and FIG. 5D shows a device having a round cross-section. In all of these variations the devices include ‘teeth’ or protrusions that are configured to roughen the joint surface, which may help with the fusion. In some variations the devices are configure to abrade, cut, and/or remove cartilage in the joint. In some variations the device is configured to abrade cartilage without substantially cutting or removing bone. In some variations the device surface is configured to abrade cut and/or remove bone from the joint.

The device may be actuated by moving it backwards and forwards (proximally and distally), by bimanual reciprocation. In some variations, such as that shown in FIG. 5E, for example, the device may also or alternatively be articulated by rotating it axially once it is in position in the joint.

In some variations the procedure for fusing the joint (e.g., facet joint) may include the use of more than one facet joint treatment devices. For example, treatment devices having different profiles (e.g., widths) may be used during the treatment. Alternatively, treatment may include selectively removing some of the bone or other tissue from the joint, which may be performed using the treatment device shown or using additional devices, including flexible bone biting devices such as the flexible ronguers described, for example in U.S. patent application Ser. No. 11/405,848, titled “Mechanical Tissue modification devices and methods” (filed Apr. 17, 2006), and herein incorporated by reference in its entirety. The same guidewire/pullwire may be used with multiple devices, as each device typically includes a distal coupler for securely coupling to the proximal end of the guidewire/pullwire, allowing it to be articulated within the joint.

Once the joint has been prepared using the device or devices, the device may be removed, and a support structure or material may be added to fuse the joint. The guidewire/pullwire may remain in position, so that it can be used to pull in or apply the material. For example, in some variations the pullwire may be used to position a cage or other mechanical support within the joint. The mechanical support may be coupled to the proximal end of the pullwire directly or indirectly (e.g., via an elongate carrier structure from which it can be released once it is positioned), and pulled into position. In some variations the pullwire may be used to pull a tube or other fluid material delivery device into position in the joint, to apply a filer material such as bone cement, bone graft material, etc. In some variations, the pullwire may be used to pull into position in the joint an expandable or fillable structure that will be implanted in the joint. For example, a mesh or porous “bag” structure may be pulled into position (and decoupled from the pullwire) and filled with appropriate fusing material (e.g., cement, etc.). In some variations a bag or balloon-like structure is pulled into position and filled.

As mentioned above, in any of the facet joint procedures described herein, all or a portion of the facet (e.g., the superior and/or inferior spinous processes) may be cut. For example, a procedure for fusing or preparing a facet joint may include a facetectomy, particularly for TLIF (Transforaminal Lumbar Interbody Fusion) procedures. The procedure may include a facet joint treatment device that is configured to saw through bone. For example, the device may include one or more cable-type saws including a distal end that is configured to couple to the pullwire as described above. As mentioned, a probe or probes may be used to place the pullwire under the facet joint. A facet joint modifying device may then be pulled in under bimanual control. Pulling the facet joint modifying device dorsally (e.g., by distal/proximal reciprocation) would result in the removal of the entire facet joint. This method may be faster than current methods which involve slow biting with ronguer-type devices.

For example, FIG. 6A illustrates a cross-section through one variation of a facet-joint modifying device that includes two bone-sawing elements 1202, 1202′. The two saw elements (which may be cables or surfaces including blades) may be separated by a spacer 1205. FIG. 6C shows a top view of one variation of a facet-joint modifying device configured to perform a facetectomy. The distal end of the device is configured to couple with the pullwire 1206, as described above. The tissue-contacting portion of the device may include two parallel cutting surfaces (which may be cables) 1202, 1202′ that are separated from each other. These two separate cutting surfaces may allow two cuts to be made through the facet joint simultaneously, permitting removal of a portion of the facet joint. This version of the facet-joint modifying device may also include one or more spacers 1205. Spacers may prevent the cutting surfaces from spreading or contracting towards each other, particularly if the cutting surfaces are cables. In some variations these spacers may be removable or separating, so that as the facet joint modifying device cuts the facet joint, pressure applied as that device is reciprocated against the bone may cause separation, breaking, or removal of the spacer. FIG. 6B illustrates a cross-section through one portion of the device having a breakable (e.g., frangible) spacer 1205.

Other facet joint modifying devices (including those shown above in FIGS. 5A-5E) may include a single tissue-modifying surface, and thus does not need a spacer.

Once the joint has been prepared using the device or devices, the device may be removed, and a support structure or material may be added to fuse the joint. The guidewire/pullwire may remain in position, so that it can be used to pull in or apply the material. For example, in some variations the pullwire may be used to position a cage or other mechanical support within the joint. The mechanical support may be coupled to the proximal end of the pullwire directly or indirectly (e.g., via an elongate carrier structure from which it can be released once it is positioned), and pulled into position. In some variations the pullwire may be used to pull a tube or other fluid material delivery device into position in the joint, to apply a filer material such as bone cement, bone graft material, etc. In some variations, the pullwire may be used to pull into position in the joint an expandable or fillable structure that will be implanted in the joint. For example, a mesh or porous “bag” structure may be pulled into position (and decoupled from the pullwire) and filled with appropriate fusing material (e.g., cement, etc.). In some variations a bag or balloon-like structure is pulled into position and filled.

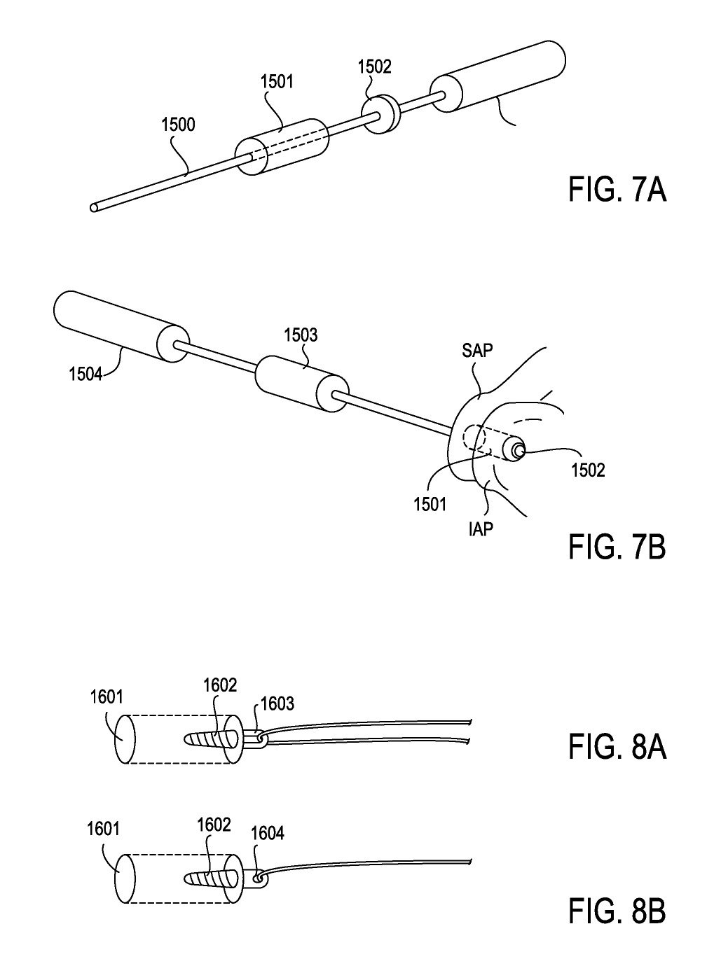

As described above, a support structure or material may be pulled into the joint space to fuse the joint. The joint may include a superior and inferior articular process, such as a facet joint, or may include two or more vertebral bodies, such as replacing or augmenting a veterbral disc. The guidewire/pullwire may be used to pull in or apply the material. In some variations, as shown in FIG. 7A, the pullwire may be used to pull a filer material such as bone cement, bone graft material, etc. into position within a joint. The material be coupled to the proximal end of the pullwire directly or indirectly (e.g., via an elongate carrier structure from which it can be released once it is positioned), and pulled into position. As shown in FIG. 7A, the pullwire 1500 is coupled to a bone graft or bone plug 1501. The method of pulling a bone plug into position via a pullwire is advantageous over conventional methods of implanting a bone plug in that the plug may be safely pulled away from a nerve root and into position, whereas in conventional methods a bone plug is typically hammered into position toward the nerve root.

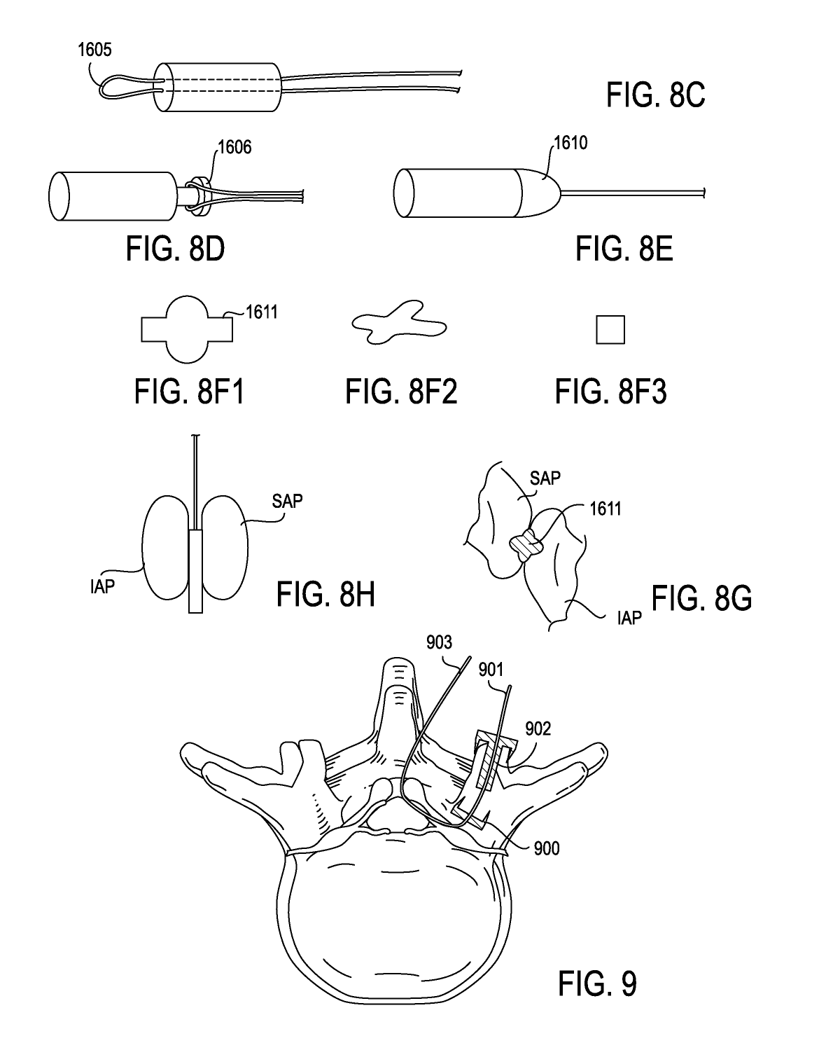

As shown, the bone plug may be cannulated such that it may be thread over the pullwire 1500. As shown, in this embodiment, the pullwire may include a stop 1502 that prevents the plug from sliding further down the pullwire and helps to apply a force to the plug as the plug is pulled into position. The stop 1502 may be crimped onto or otherwise connected to the pullwire. The stop may have a diameter equal to the outer diameter of the bone plug, or may alternatively have a larger or smaller diameter. The stop may be positioned distal to the plug, or may alternatively be positioned proximally to the plug. As described above, the plug may be pulled into position by pulling on the pullwire, thereby applying a force to the plug via the stop coupled to the pullwire. As shown in FIG. 8C, the stop 1605 may be a knot in the pullwire. For example, the pullwire may be made out of a suture or other flexible material. Alternatively, the pullwire may be a metallic material, such as Nitinol. As shown in FIG. 8C, the pullwire may be cut to release the plug from the pullwire.

The plug may be designed such that the diameter of the plug is slightly larger than the hole or slot created for the plug within the joint. Therefore, when the plug is pulled into position, it may be wedged and therefore fixed between the joint surfaces. By wedging the plug between the joint surfaces, the plug will remain in position while the bone grows and the joint fuses together. As shown in FIG. 7B, a sliding impact weight 1503 may also be coupled to the pullwire. As shown, the weight 1503 may be coupled to the pullwire on the opposite side of the plug 1501 from the stop 1502. Once the plug is pulled into position the pullwire may be pulled to pull the plug into position. The weight may be pulled along the pullwire and slammed into a handle 1504 fixed to the pullwire. As the weight impacts the handle, an increased force will be applied to the pullwire and therefore to the plug, pulling the plug into position.

Alternatively, as shown in FIGS. 8A and 8B, the plug 1601 may further include a screw 1602. The screw 1602 may be thread into the bone plug. The screw may be metallic and/or may be an absorbable or bioresorbable material such as PLLA, PGA, or PLDA. The screw may also include a coupling mechanism 1603 or 1604. The pullwire is removably coupled to the coupling mechanism. For example, as shown in FIG. 8A, the pullwire may be wrapped through the loop 1603. Alternatively, the pullwire, as shown in FIG. 8B, may be removably coupled to the coupling mechanism in an end-to-end fashion. For example, the coupling mechanism 1604 may include a geometry adapted to receive the geometry of an end of the pullwire.

As shown in FIG. 8D, the plug may be shaped to include a coupling mechanism 1606. For example, the plug may be molded or machined to include coupling element 1606. The pullwire is removably coupled to the coupling mechanism. For example, as shown, the pullwire may be wrapped around the coupling mechanism. Alternatively, the pullwire may be removably coupled to the coupling mechanism 1606 in an end-to-end fashion. For example, the coupling mechanism may include a geometry (not shown) adapted to receive the geometry of an end of the pullwire.

As shown in FIG. 8E, the plug may be shaped to include a tapered end 1610. The end may be configured to aid in the delivery and centering of the plug between two bones. As shown in FIG. 8F, the plug may be shaped to prevent rotation of the plug between the two bones. For example, as shown in FIG. 8G, the plug has a substantially circular cross section with at least one wing 1611. The wing prevents rotation of the plug while in position.

As shown in FIG. 9, in addition to a dowel pin (e.g. an allograft dowel pin) being thread over the guidewire and pulled into position, the system may also include a bottom cap 900 and a top cap 901. These caps may function to hold the dowel pin 902 into position and/or prevent the dowel pin from working out of the bone. The caps may also function to grip and bind the first joint surface to the second joint surface. For example, the first joint surface may be a superior articular process (SAP) and the second joint surface may be an inferior articular process (IAP) of a facet joint. In some embodiments, the bottom cap may be pulled into position using the guidewire 903 and then subsequently pulled into the bottom (anterior) joint surfaces. The top cap may then be thread over the guidewire in the opposite direction and subsequently pushed into the top (posterior) joint surfaces. The caps may function to restrict or minimize movement of the joint such that the dowel pin may remain in position and successfully fuse the joint surfaces together. As shown, the bone graft may be an integral part of the top cap. Alternatively, the bone graft may be inserted prior to the cap, or after the cap through a hole in the cap. In some embodiments, a band (as described in greater detail below) may be wrapped around the exterior of the joint, in addition to the implant of the top and bottom caps. In this embodiment, the caps may function to fix the band in place around the joint.

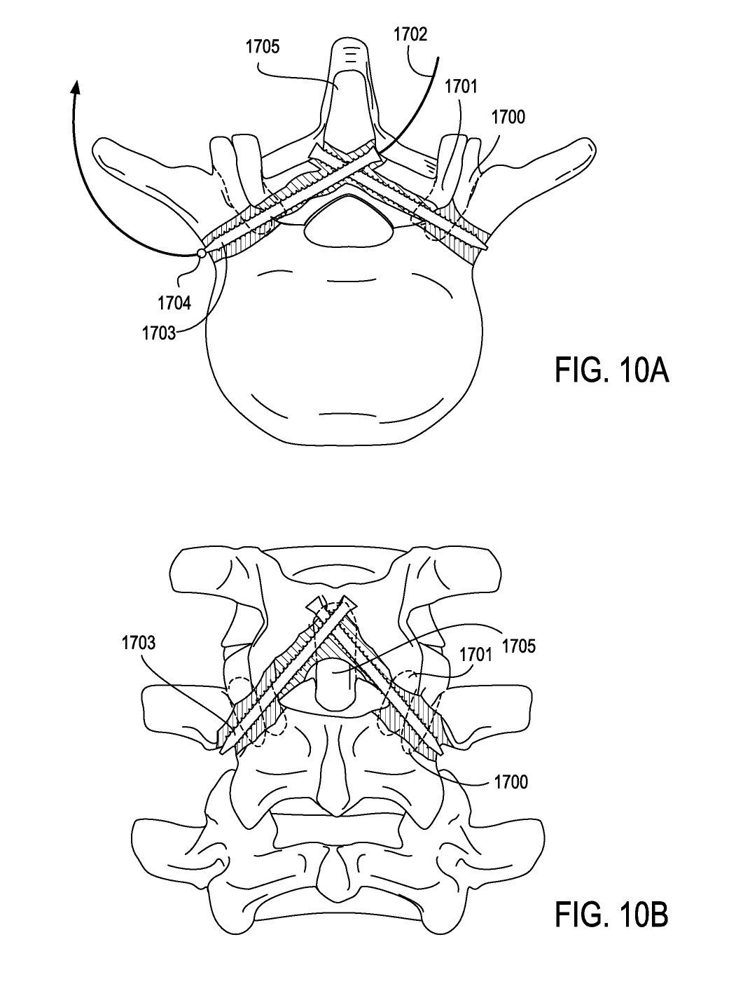

As described above, a support structure, such as a screw or pin, may be pulled into the joint space to fuse the joint. For example, as shown in FIGS. 10A and 10B, the joint may be a facet joint and include a superior articular process 1700 and inferior articular process 1701. The guidewire/pullwire 1702 may be used to pull in a screw 1703 or a pin (i.e. a non-threaded screw). The screw may be coupled to the proximal end of the pullwire directly or indirectly (e.g., via an elongate carrier structure from which it can be released once it is positioned), and pulled into position. As shown in FIG. 10A, the pullwire 1702 is coupled to screw 1703 at point 1704 such that the screw may be pulled down into the facet joint via the spinous process 1705 or lamina. Screw 1703 is a translaminar facet screw, which are typically used in a TLIF procedure. As described below, the screw may alternatively be a transfacet or transpedicular screw, or any other suitable screw or pin. In some variations, the screw may be cannulated such that it may be fed over the pullwire. Alternatively, the screw may be crimped onto or otherwise coupled to the pullwire. The screw and/or pullwire may include a coupling mechanism as described above.

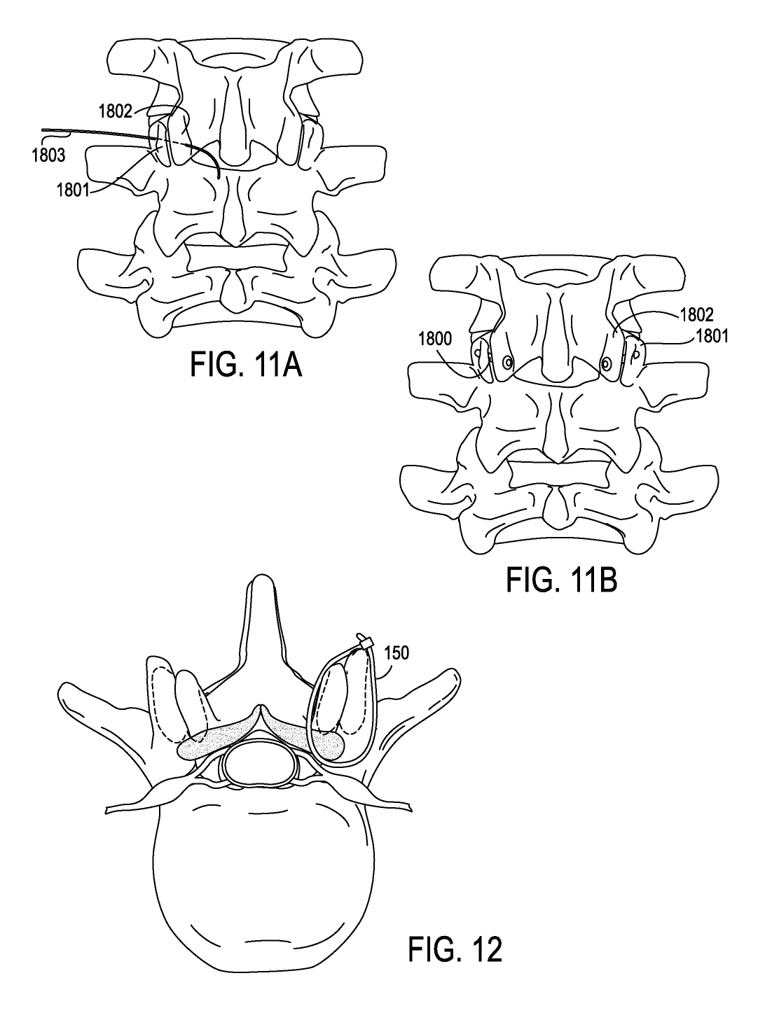

A support structure, such as a transfacet screw or transfacet pin 1800, may be pulled into the joint space to fuse the joint. For example, as shown in FIGS. 11A and 11B, the joint may be a facet joint and include a superior articular process 1801 and inferior articular process 1802. The guidewire/pullwire 1803 may be used to pull in pin 1800. The pin may be coupled to the proximal end of the pullwire directly or indirectly, as described above, and pulled into position. As shown in FIG. 11A, the pullwire 1803 is fed into position into an inferior articular process 1802 and then into a superior articular process 1801. Alternatively, the pullwire may be thread in the opposite direction. The pin 1800 may then be coupled to the pullwire and pulled into the joint. The pin or screw may be fixed to the bone via mechanical structures and/or the geometry of the pin for example. Alternatively the pin may be fixed with glue or cement.

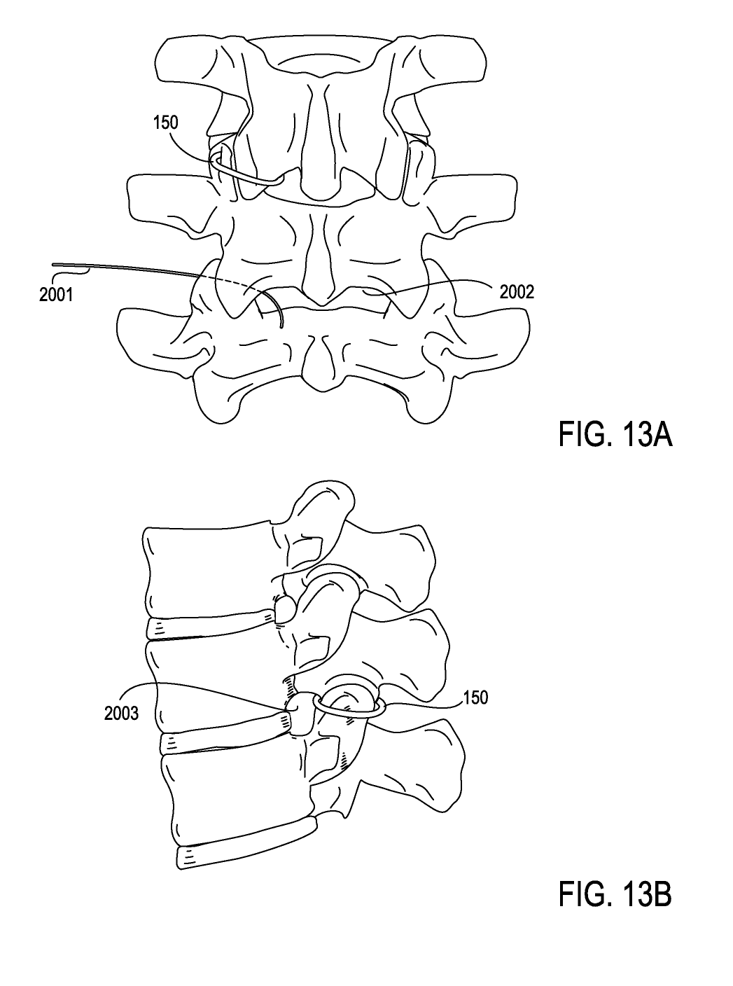

In an alternative variation, the support structure may be a cinch or band wrapped around a joint, such as a facet joint, as shown in FIG. 12. For example, a thin flat element 150 may be pulled tightly around the facet complex. Other variations include a totally implanted and completely biodegradable element 150. In some embodiments, the joint may be prepared (i.e. abraded) for fusion as described above and then the wrap 150 may be coupled to the facet joint to compress the bony (and in some instances bloody) surfaces against one another to promote fusion of the joint. In some embodiments, a tissue modification device may first be fed around the facet joint by pullwire 2001, for example, as shown in FIG. 13A through the interlaminar window 2002, around the facet joint, and out through a spinal foramen 2003 (FIG. 13B). Once in position, the tissue modification device may be bimanually reciprocated against the tissue of the facet joint to modify and/or remove tissue. The tissue modification device may perform an entire decompression or alternatively may create a trough around the outer surface of the facet joint and prepare the outer surface of the facet joint to receive band 150. Additionally, a bone plug, as described above may be pulled into the facet joint prior to the placement of band 150. Band 150 may be coupled to a pullwire and pulled into position as with the other devices as described above. A probe, as described herein, may be used to position the pullwire and/or the band 150. The probe may be used in an ipsilateral approach and/or from a contralateral approach.

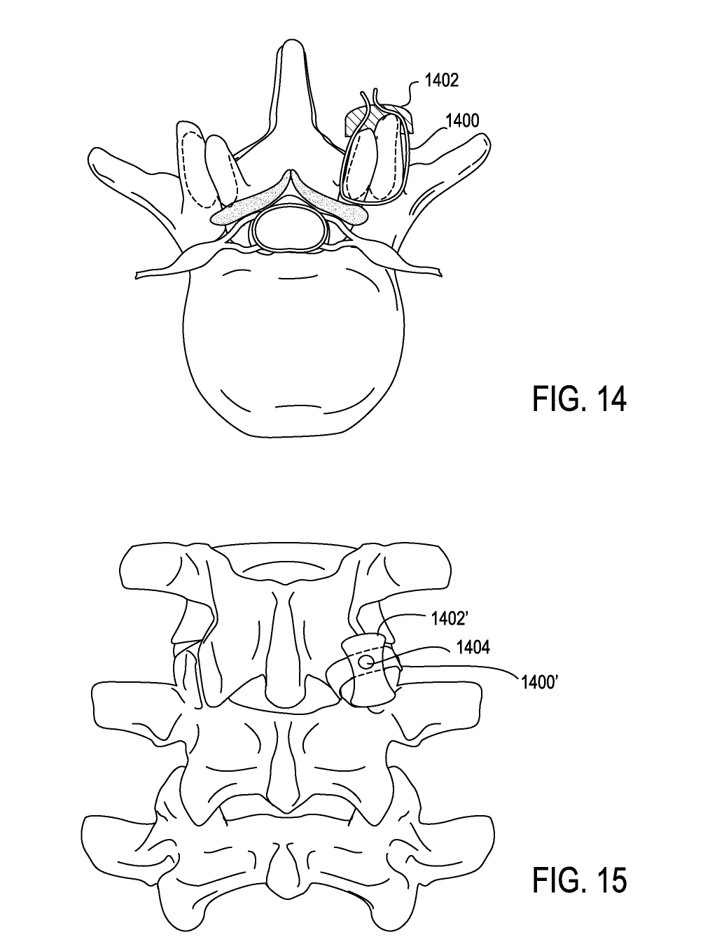

FIG. 14 illustrates an embodiment of a strap device having a cap. Various embodiments of strap and cap devices will be described in detail below. As shown, the strap 1400 may function to wrap tightly around a joint, such as a facet joint. In these embodiments, the devices function to prevent or minimizes movement of the spine in at least one of the following directions: flexion/extension, torsion, and lateral bending. The strap and/or cap may function to restrict or minimize movement of the joint in order to facilitate or promote fusion of the joint. The strap may be a metallic material, such as titanium, stainless steel, nitinol, etc. Alternatively, the strap may be a plastic (such as nylon or PEEK) or made or a textile or mesh fabric or braided material. In some embodiments, the strap may be a biodegradable material such as PLA or PGA. The cap 1402 may also be made from any of the materials described above. Alternatively, the cap may be machined or formed out of bone, allograft, or other suitable biologic material.

As described above, the strap may be fed around the facet joint and pulled into position using a guidewire as described in detail above. The strap may be pulled into position from either an ipsilateral approach or a contralateral approach. Prior to pulling in the strap, a shaver or other suitable tissue modification device may be pulled into position using the guidewire. The tissue modification device may be used to shave away ligament and/or bone on the anterior surface of the facet joint. In some embodiments, a groove, trough, and or channel may be cut. The trough may be sized and configured to receive the strap. Once the strap is pulled into position, the cap may be thread over the guidewire and/or over the free ends of the strap. The cap may function to cinch the strap tightly around the joint and fix the strap into position and hold the tension of the strap. In some embodiments, a tensioning device may be used to pull the ends of the strap tight and tension the strap around the facet joint. The cap may prevent the strap from loosening overtime. In some embodiments, once the free ends of the strap are pulled through the cap, the excess length may be cut, or otherwise removed, from the strap.

FIG. 15 illustrates an alternative embodiment of a strap device having a cap. As shown in FIG. 15, the cap 1402′ is placed across the facet joint in an alternative orientation. In this embodiment, the cap is positioned in the superior/inferior direction. The cap in this orientation may function to prevent the movement of the joint in the flexion/extension directions. The combination of the strap 1400′ and the cap preferably prevent movement of the spine in each possible direction, including: flexion/extension, torsion, and lateral bending. In some embodiments, the facet joint may require preparation, such as shaping, prior to the placement of the cap. For example, in some embodiments, the joint may be large or hypertrophied. Bone may be drilled, shaved, cut, or bitten away. For example, a Rongeur or other bone cutting or biting device may be used to remove bone and/or ligament to shape and prepare the facet joint for the cap and/or the strap. In some embodiments, the cap and/or the strap may further include a hole positioned over the center of the joint. This hole may be sized and configured to receive a dowel pin and or screw. The pin or screw 1404 may function to secure the strap and/or cap to the bone and/or may facilitate and/or promote fusion of the joint. Alternatively, a bone paste, such as one including bone morphogenetic proteins, may be placed between the surfaces of the joint to promote fusion of the joint. The paste may be applied prior to positioning the strap or may alternatively be applied through the hole in the strap and/or cap as described above.

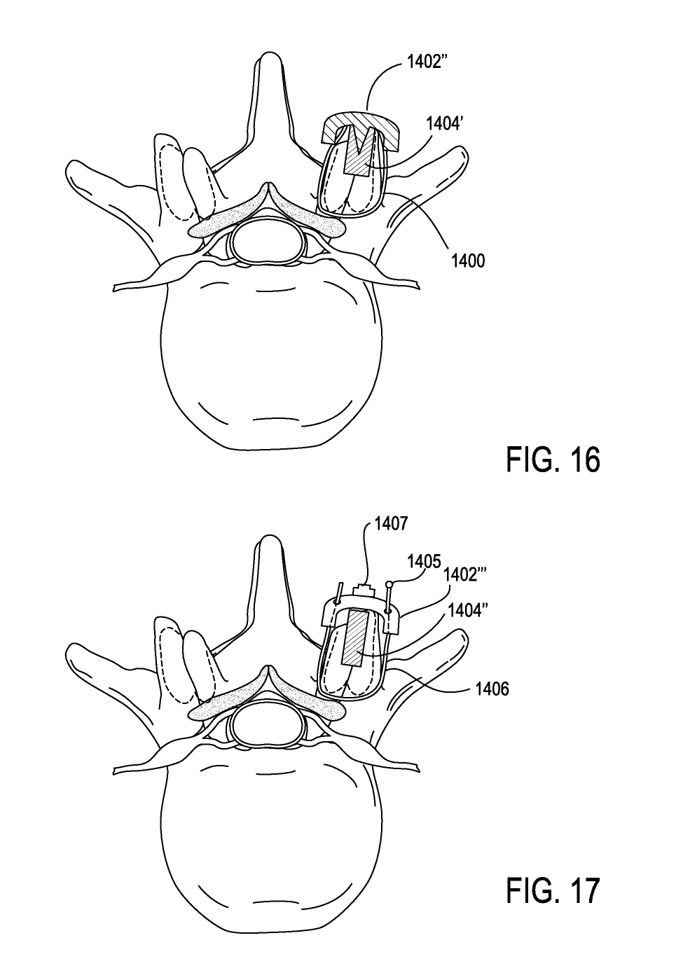

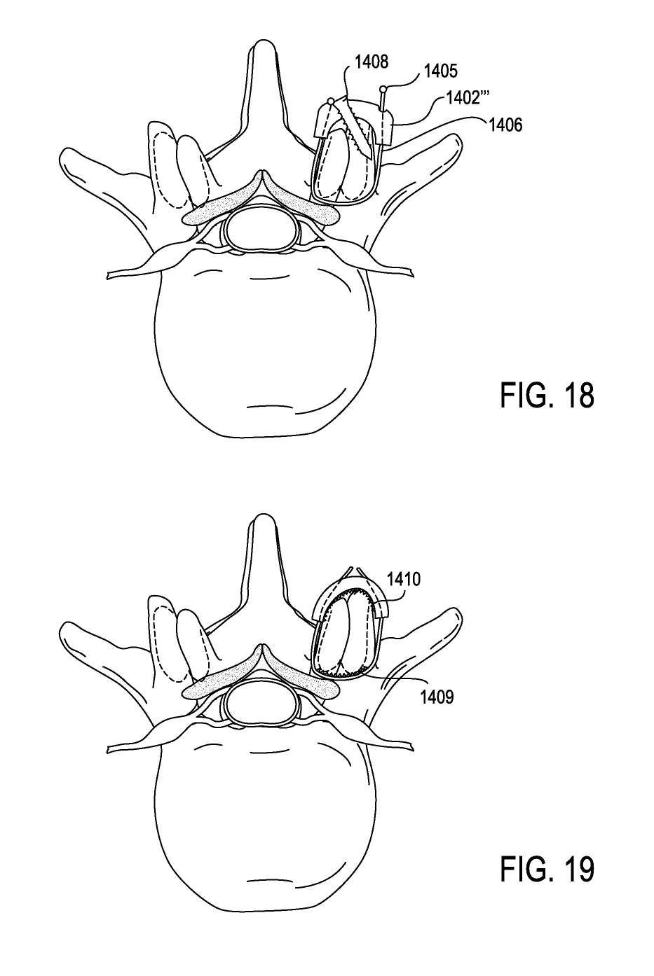

FIG. 16 illustrates an alternative embodiment in which the cap 1402″ is configured to secure to a dowel or bone graft 1404′. As shown, the cap may include a spike or screw configured to receive and secure a dowel bone, such as a bone graft. FIG. 17 illustrates an alternative embodiment of the cap including features to lock the ends of the strap into place. For example, as shown, a first end of the strap includes an enlarged end 1405. The enlarged end is configured to be received by a notch in the cap. As the opposite end of the strap 1406 is pulled taught around the joint, the enlarged end will be pulled into the notch and fixed into position. The opposite end of the strap may be fixed into place by an eccentric cam locking mechanism 1407, for example. The opposite end of the strap may be pulled through the cap, and then locked down into place by rotating the cam. In an alternative variation, as shown in FIG. 18, the cap includes a locking screw 1408 to lock the opposite end of the strap into position. The locking screw may further function to screw into the joint and fix the cap and/or strap into position. In some embodiments, the screw may be positioned across the joint to fix the joint and/or promote or facilitate fusion.

FIG. 19 illustrates an alternative embodiment of a strap and cap device. As shown, the strap includes a first anchor mechanism 1409. The first anchor may be positioned on the strap such that it is pulled into position and grabs into the ligament and/or bone at the anterior portion of the facet joint. As shown in FIG. 19, the cap may also include an anchor mechanism 1410. The cap anchor mechanism may grab onto the superior articular process alone, or onto both the inferior articular process and the superior articular process. The anchor mechanisms may include any combination of blades, teeth, or other suitable abrasive surfaces. In some embodiments, the anchor mechanisms may alternatively include an adhesive material or solution such as bone cement.

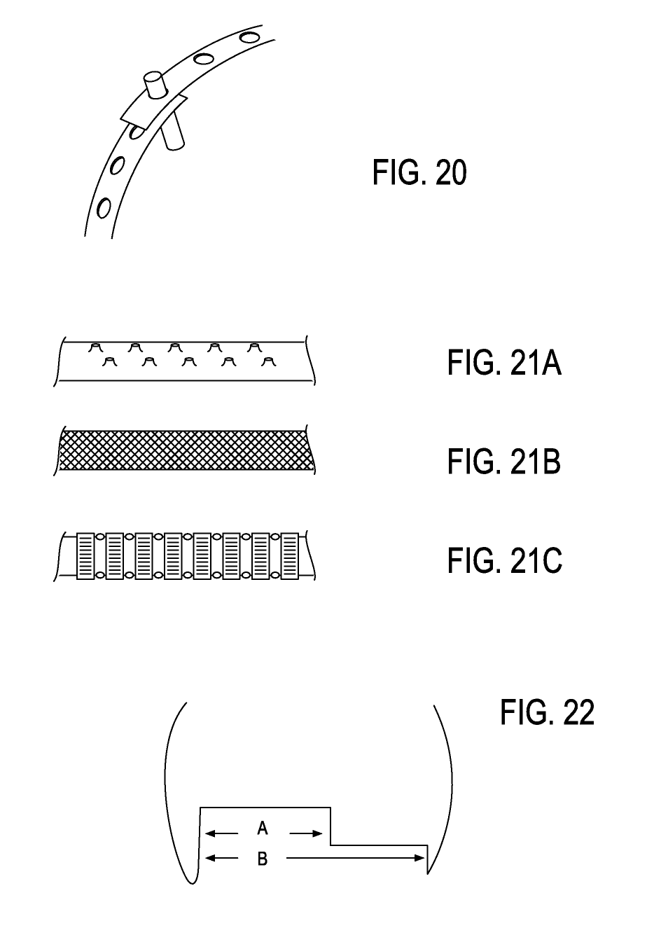

FIG. 20 illustrates an alternative embodiment of a strap. In this embodiment, as shown the strap may be configured to include a series or holes or perforations. As the strap is tightened around the joint, a hole on the first end of the strap may be placed over a bone dowel or screw. Then, as the strap is tightened further, a hole on the second end of the strap may also be placed over the bone dowel or screw. In this embodiment, the end of the bone dowel or screw protrudes from the joint such that the strap may be placed over and fixed onto the dowel. In this embodiment a cap or screw (not shown) may be placed over the straps and dowel and/or fixed to the strap and dowel to fix them into position.

FIGS. 21A-21C illustrate various embodiments of the strap material. As described above, the strap may include anchor mechanisms such that the strap is fixed to and/or “grabs” onto the bone of the joint and/or ligament adjacent to the joint. In a first embodiment, as shown in FIG. 21A, the strap (or a portion thereof) is serrated. For example, the strap may include “volcano” shaped blades. In an alternative embodiment, as shown in FIG. 21B, the surface of the strap may be knurled or otherwise abrasive. For example, the surface of the strap may include abrasive particles. Alternatively, as shown in FIG. 21C, the strap may be a runged device. In some embodiments, the rungs, or a portion thereof, may include blades. The blades may function to cut into bone and/or ligament and fix the strap into position.

FIG. 22 shows a method of preparing a bone surface for a band or strap. In some embodiments, multiple tissue modification devices, such as rasps or shavers, may be pulled into position around the joint. As shown, a first device having a width A, for example, may be used to create a trough in the bone having a width A. A second device having a width B, may then be used to create a second, wider trough having a width B. The devices may be used in any suitable order. As shown, the troughs may be positioned toward a single side of the bone (left side, as shown). Alternatively, the troughs may be centered over one another. In some embodiments, trough B may function to decompress and alleviate a nerve root, while trough A may function to receive the band or strap fusion device and hold the device in place. As described above, in some embodiments, a tissue modification device may first be fed around the facet joint by pullwire through an interlaminar window, around the facet joint, and out through a spinal foramen. Once in position, the tissue modification device may be bimanually reciprocated against the tissue of the facet joint to modify and/or remove tissue. The tissue modification device may perform a decompression and/or may create a trough around the outer surface of the facet joint and prepare the outer surface of the facet joint to receive band.

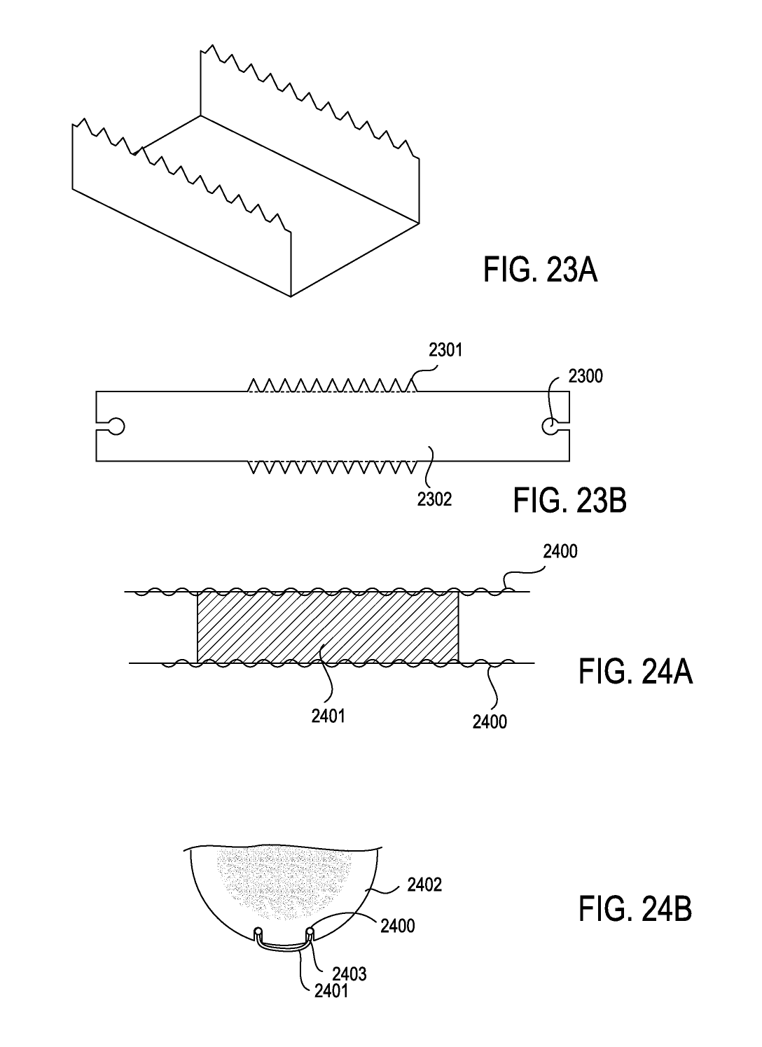

FIGS. 23A and 23B illustrate a method of manufacturing a strap device having an anchoring mechanism. In this embodiment, a material such as metal or plastic may be stamped, wire EDM'ed, or otherwise machined into a shape, as shown in FIG. 23B. The teeth or serrated edges 2301 of the strap may then be bent or folded such that they are perpendicular to the base 2302 of the strap. Alternatively, the teeth may be at any other suitable angle to the base of the strap. As shown in FIG. 23B, one or both of the ends of the strap may include a guidewire coupler 2300. The coupler may function to couple the strap to a guidewire or pullwire such that the guidewire may pull the strap into position around the facet joint.

FIG. 24A illustrates an alternative embodiment of a strap device. In this embodiment, the device includes two cutting wires 2400, such as Gigli saws. The strap 2401 may be coupled to the cutting saws such that it is positioned between them. As shown in FIG. 24B, the cutting saws are pulled into position at the base of the joint 2402. As the saws are reciprocated in a bimanual fashion (as described above) the saw will cut grooves 2403 into the anterior portion of the facet joint. As the cutting saws move into the bone, they will pull the strap in behind them as shown. This may function to lock the strap into place around the joint.

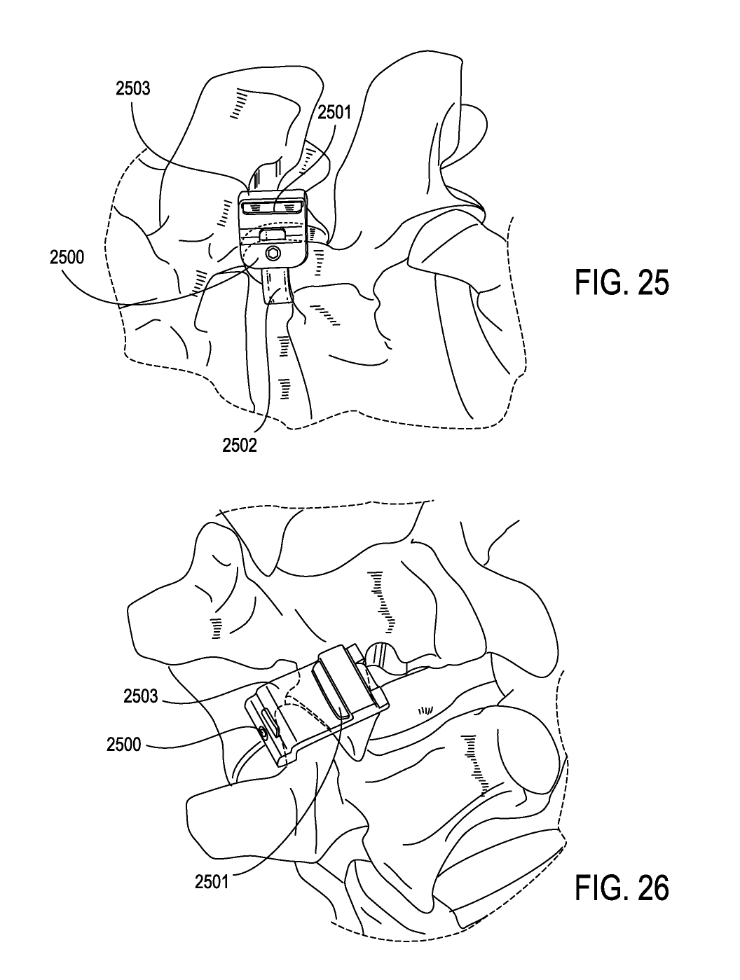

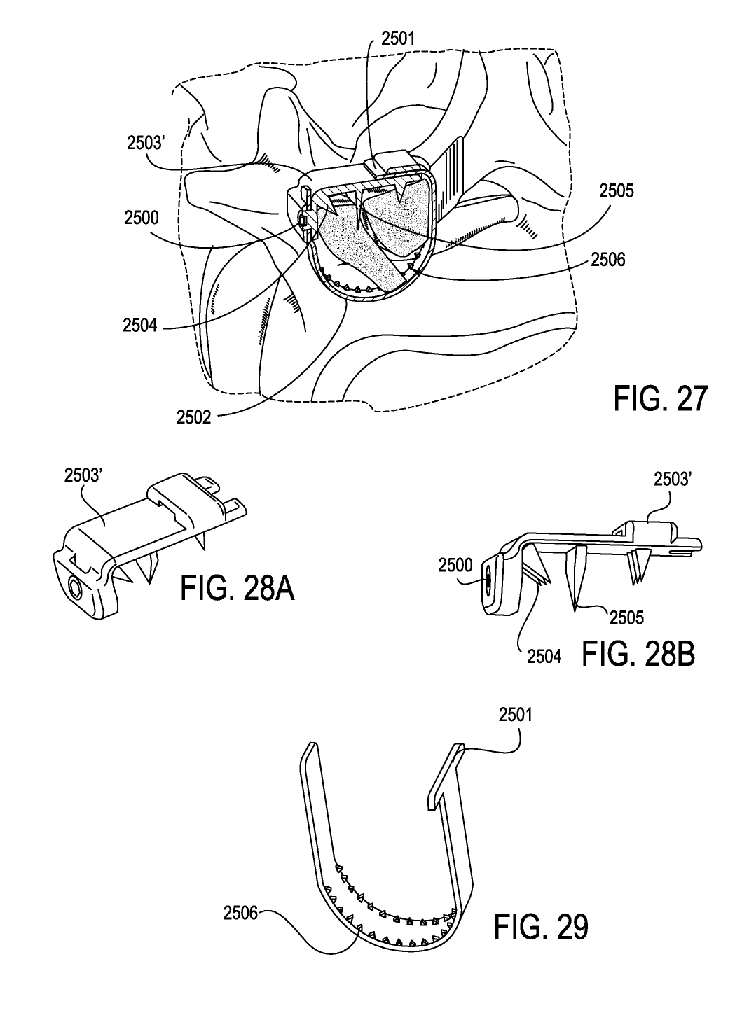

FIGS. 25-29 show three dimensional views of an embodiment of a strap and cap device as described herein. In this embodiment, as shown in FIG. 25, the cap 2503 includes an active locking mechanism 2500 and a passive locking mechanism. The passive locking mechanism may function to catch the shaped end 2501 of the strap 2502 as the strap is pulled and tensioned around the facet joint. The strap may be pulled and tensioned around the facet joint by pulling on the opposite end of the strap (not shaped end). The strap may be pulled on directly, or a guidewire may be coupled to the strap. A user may pull and tension the guidewire to pull and tension the strap around the facet joint. Once the shaped end of the strap is locked in place by the passive locking mechanism of the cap, the active locking mechanism may be locked onto the opposite end of the strap. FIG. 27 shows an implanted strap and cap device in cross section. As shown, the cap 2503′ may further include a spike 2504 or a plurality of spikes that may function to pierce and/or “grab onto” the bony surfaces of the facet joint. Optionally, the cap may further include a joint spike 2505 that may be placed between the articulating surfaces of the joint. In some embodiments, the joint spike may be an allograft material or may include a bone paste or cement on the surface of the spike to promote fusion of the articulating surfaces of the facet joint. Furthermore, as shown in FIG. 27, the strap may include an anchoring mechanism 2506. In this embodiment, the strap may include a plurality of teeth that may function to pierce and/or “grab onto” the bony surfaces of the facet joint. In some embodiments, the anchoring mechanism may be anchored to ligament or other soft tissue adjacent to the joint. FIGS. 28A-29 further illustrate the features of the strap and cap device as described above.

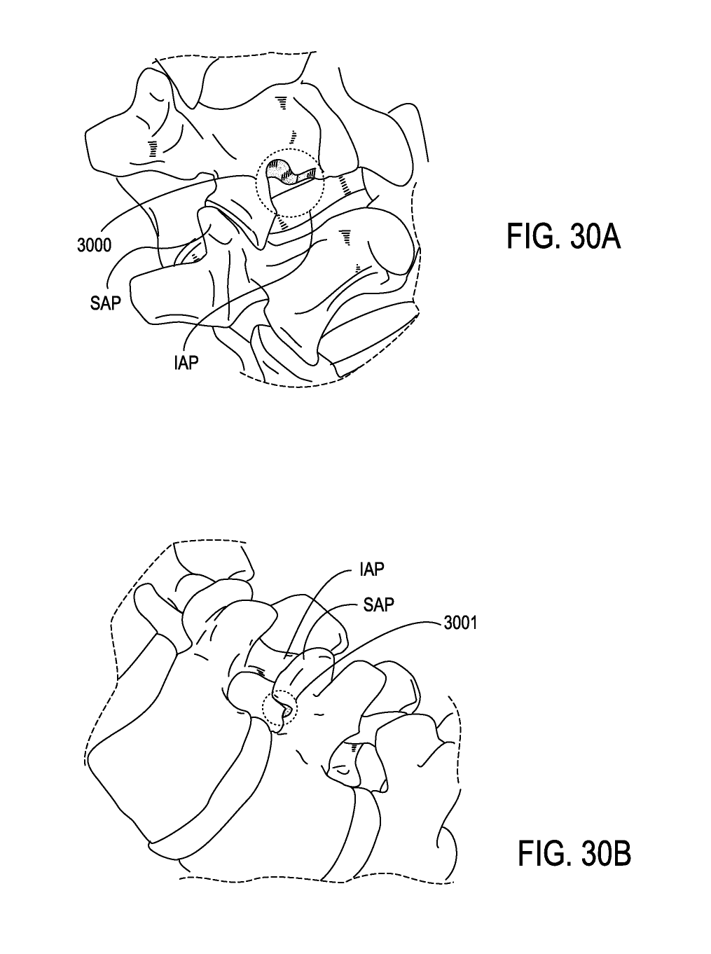

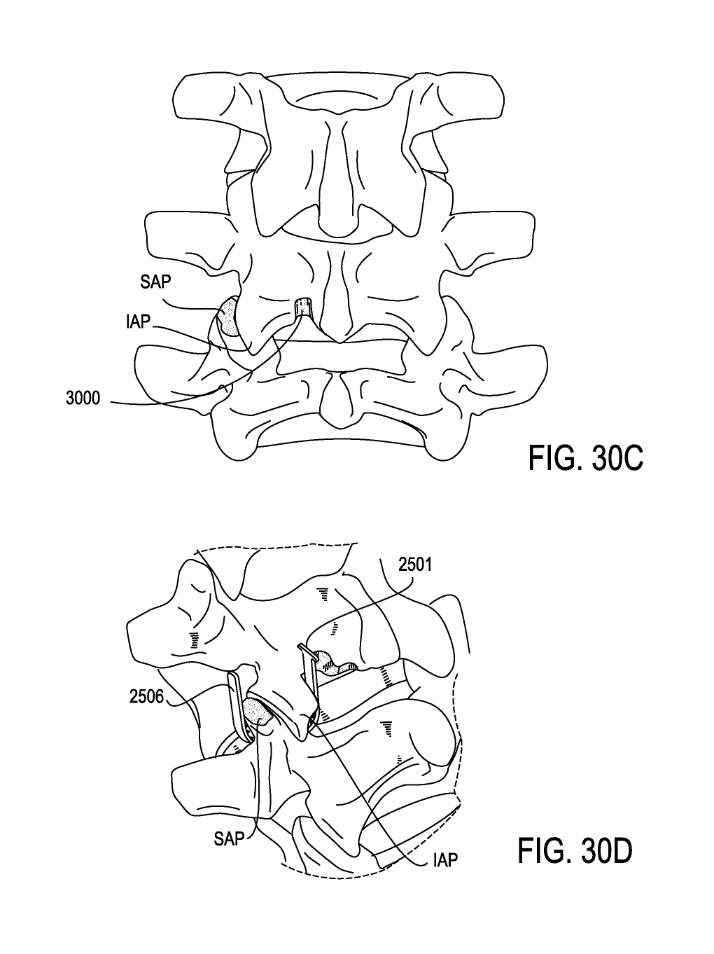

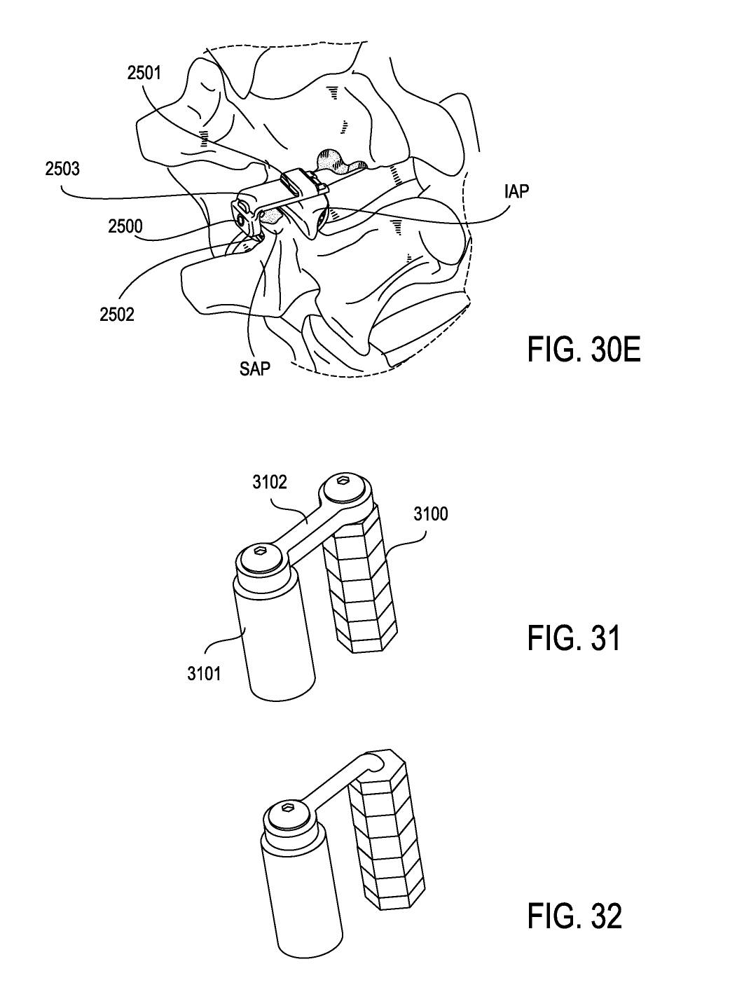

FIGS. 30A-30E show a method of implanting an embodiment of a strap and cap device as described herein. In some embodiments, as shown in FIG. 30A, a laminotomy 3000 may be performed. This step includes removing bone from the lamina of a vertebra. The laminotomy may be performed using a Rongeur, such as a Karrison Rongeur, or may alternatively be performed using any other suitable bone cutting, drilling, or biting device. In some embodiments, as shown in FIG. 30B, a decompression 3001 may be performed. As described above, in some embodiments, a tissue modification device (not shown) may fed around the facet joint by a pullwire through an interlaminar window, around the facet joint, and out through a spinal foramen. Once in position, the tissue modification device may be bimanually reciprocated against the tissue of the facet joint to modify and/or remove tissue. The tissue modification device may perform a decompression and/or may create a trough around the outer surface of the facet joint (as shown in FIG. 30B) and prepare the outer surface of the facet joint to receive the strap and cap device. In some embodiments, as shown in FIG. 30C, the posterior surface of the joint may be prepared to receive the strap and cap device. Specifically, as shown, portions of the superior articular process (SAP) may be removed to shape and prepare the surface of the joint to receive the strap and cap device. In some embodiments, additionally or alternatively, the inferior articular process (IAP) may be modified and/or prepared. In some embodiments, the articulating surfaces of the joint may be roughened or otherwise modified to promote fusions of the surfaces. For example, the outer cancellous bone or the articulating cartilage of the joint may be removed. By removing this bone from each articulating surface and thereby approximating a bloody bone surface to bloody bone surface, fusion is more likely to occur. Additionally bone cement or bone paste (including BMPs) may be placed within the joint.

As shown in FIG. 30D, once the joint and surrounding areas of the spine have been adequately prepared, the strap may be implanted. As described above, the strap may be pulled around the facet joint directly or by way of a guidewire. As shown, the strap may be implanted through the laminotomy and/or interlaminar window, around the facet joint, out through the neural foramen and then back up (posterior). Once the strap is positioned, the cap may be placed, as shown in FIG. 30E. Alternatively, in some embodiments, the cap may be placed first, and the strap may be fed through the cap. As shown in FIG. 30E, as the strap is tensioned around the facet joint, the shaped end of the strap may be fixed in position by the passive locking mechanism of the cap. The opposite end of the strap may then be locked into place by screwing down or otherwise locking the active locking mechanism. In some embodiments, the excess free length of the strap may be cut and removed.

As described herein are devices, systems and method for performing spinal fusion surgery, in particular a less invasive posterior column fusion or soft fusion, by way of an interspinous fusion or spinous process fixation, for example. Additionally, facet joint fixation may also be performed—either bilaterally or unilaterally. In some embodiments, a spinal decompression procedure may also be performed, since it may enhance success rates of the spinal fusion procedure.

These systems and methods may be particularly useful for percutaneous treatments of one or more body region. However, it should be understood than any of the devices, methods and systems described herein may be used as part of an “open” surgical procedure in which access to a body region is created through an opening in the tissue (e.g., by movement or removal of tissue). Any of the systems and devices described may be performed as part of a procedure that is at least partially open. Partially percutaneous procedures may also be performed using these devices, systems and methods.

FIGS. 31 to 33B show an exemplary embodiment of a facet fixation system. FIG. 31 illustrates a first embodiment of a facet fixation device. As shown, the device includes a first plug or screw 3100 that is sized and configured to be implanted in a first portion of the spine and a second plug or screw 3101 that is sized and configured to be implanted in a second portion of the spine. The device may also include an optional interconnect element 3102 that connects the first plug or screw to the second plug or screw. In some embodiments, as shown in FIG. 31, the interconnect may be coupled to the plugs via a screw that couples an end of the interconnect to the plug. Alternatively, as shown in FIG. 32, the interconnect may comprise an L-shaped member, wherein one arm of the “L” may be coupled to a first plug, while the end of the second arm of the “L” may be coupled to the second plug with a screw as described above. In some embodiments, the plugs (or screws) may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the plugs (or screws) may be metallic or plastic. For example, the plugs (or screws) may be made out of polyetheretherketone (PEEK). As shown, the plugs (or screws) may define each a channel that are sized and configured to receive a screw or other coupling mechanism. In some embodiments, the plugs may be made out of the same material, while in alternative embodiments, they may be made out of different materials. For example, the first plug may be made out of an allograft material, while the second plug may be made out of a plastic material. In some embodiments, at least one of the plugs may function as a bumper or distraction element. In this embodiment, the plug may be made out of a plastic material for example. The bumper may function to hold a joint or portion of the spine in a specific configuration. For example, the bumper may hold a facet joint in a distracted or modified position such that the spine is “flattened” out, for example.

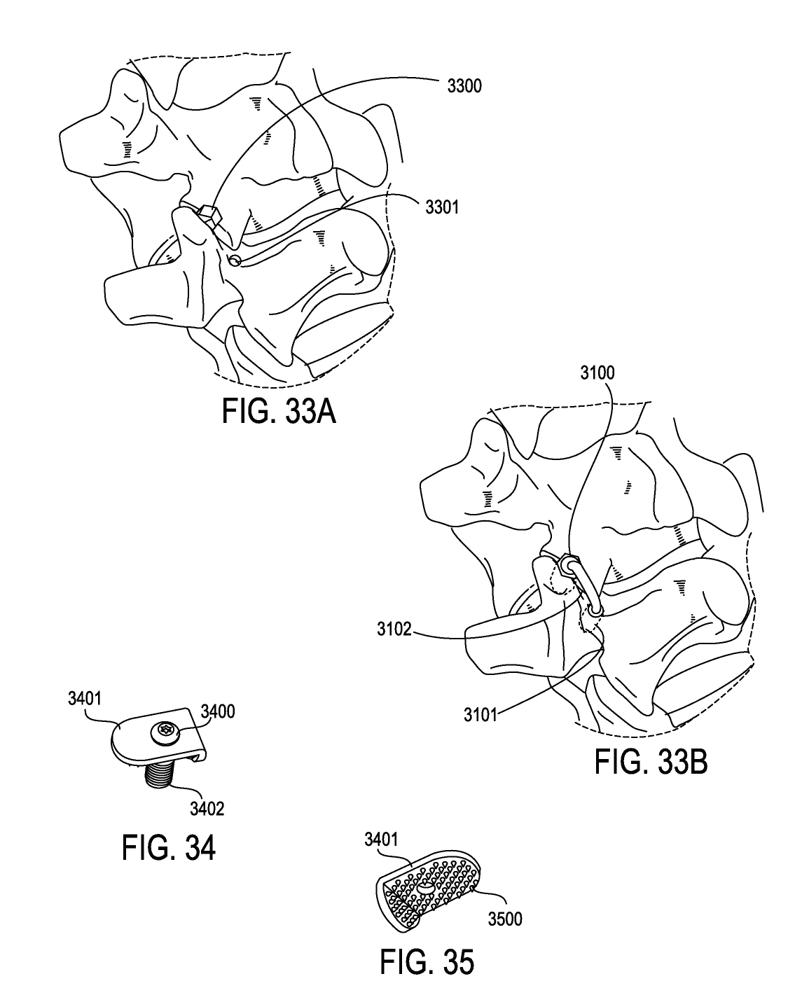

FIGS. 33A and 33B illustrate an exemplary method of implanting a facet fixation device. As shown in FIG. 33A, holes may be drilled or punched into the spine. The holes may be sized and configured to receive the first and second plugs or screws of the fixation device. For example, as shown, a first inter-facet hole 3300 may be created within the face joint. The hole may be drilled, punched, or broached. As shown, the hole may have a hexagonal cross-section. A noncircular cross section may prevent the implanted plug or screw from rotating within the hole. Alternatively, the hole may have a rectangular, oval, circular, or any other suitable geometry cross section. A second hole 3301 may be created through the lateral edge of the pars and into the medical wall of the pedicle, for example. Alternatively, the second hole may be created in any other suitable location. In some embodiments, as shown in FIG. 33B, a first plug 3100 comprising an allograft material may be implanted into the inter-facet hole and a second plug 3101 may be implanted into the inter-pedicular hole. In some embodiments, the second plug may be a bumper as described above. The position and/or configuration of the second plug may move and/or hold the spine in extension, for example. An optional interconnect 3102 may then be coupled to the first and second plugs. The interconnect may function to prevent the inter-facet plug from “backing-out” of the hole and/or the spine. The interconnect may also help to reduce bending, torsion, flexion, and/or extension of the spine or a portion of the spine. In some embodiments, the length of the interconnect may move and/or hold the spine in extension, for example.

In some embodiments, the fixation device includes a fixation screw configured to be implanted into a facet joint and a facet cap configured to couple to the fixation screw and to couple to a medial aspect and a lateral aspect of a facet joint. FIGS. 34 and 35 show an exemplary embodiment of such a facet fixation system. As shown in FIG. 34, the facet fixation device includes a fixation screw 3402 sized and configured to be implanted into or adjacent to a facet joint, a facet cap 3401 configured to couple to the fixation screw and to couple an inferior acricular process (IAP) and a superior articular process (SAP) of a facet joint, and an optional coupling screw 3400 configured to couple the facet cat to the fixation screw. In some embodiments, the fixation screw and/or the facet cap may be made out of a bone graft or allograft material. Alternatively, the fixation screw and/or facet cap may be made out of plastic or metal as described above. As shown in FIG. 35, in some embodiments, the facet cap may include coupling members 3500. For example, as shown, the coupling members may be spikes or protrusions sized and configured to dig into or catch onto bone thereby aiding in the coupling of the facet cap to the facet joint. Alternatively, the coupling members may be an abrasive surface, abrasive particles, or may include material such as bone cement or bone graft (including BMP for example).

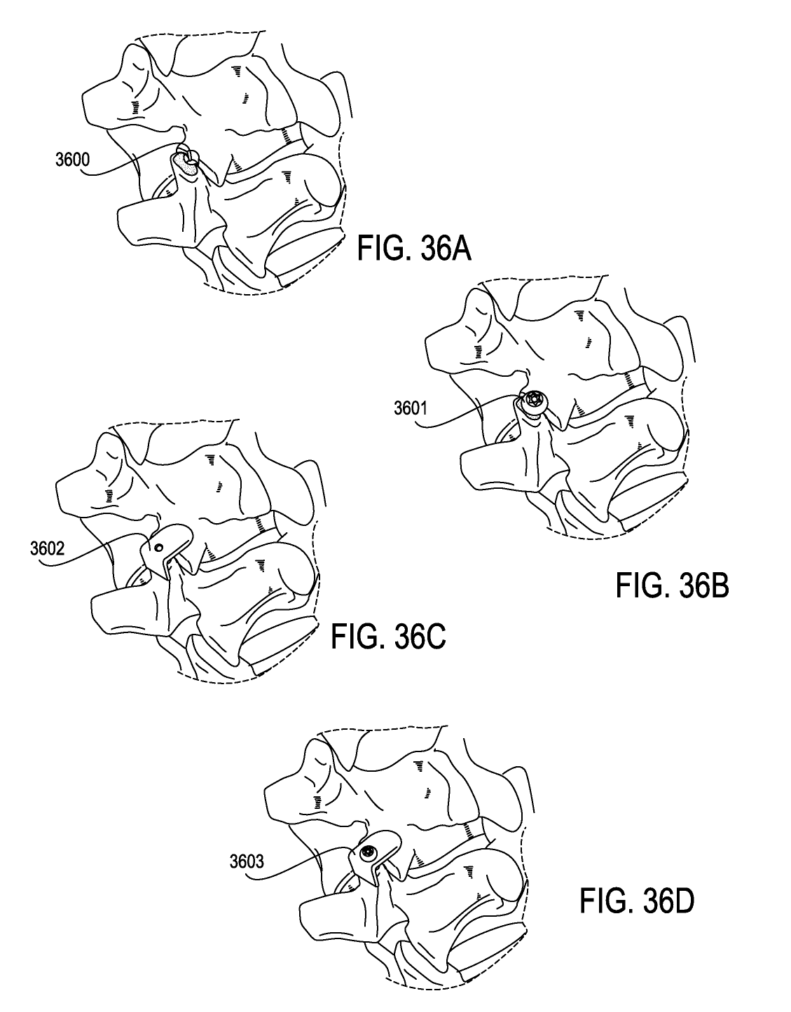

FIGS. 36A-36D illustrate a method of implanting the facet fixation device as described in reference to FIGS. 34 and 35. As shown in FIG. 36A, a hole 3600 may be created within a facet joint. The hole may be drilled, punched, broached, etc. As described above, the hole may have a circular or noncircular cross section. As shown in FIG. 36B, a fixation screw 3601 may be implanted into the inter-facet hole. As shown in FIG. 36C, a facet cap 3602 may be placed over the fixation screw and coupled to at least two portions of the facet joint. For example, the facet cap may be coupled to a portion of the IAP and a portion of the SAP. In some embodiments, the facet joint, or a portion thereof, may be prepared (e.g. resurfaced) as described in reference to FIG. 30C. As shown in FIG. 36D, an optional coupling screw 3603 may be implanted. The coupling screw may function to connect the facet cap to the fixation screw. In some alternative embodiments, a coupling screw may be configured to couple the facet cap to a portion of the facet joint.

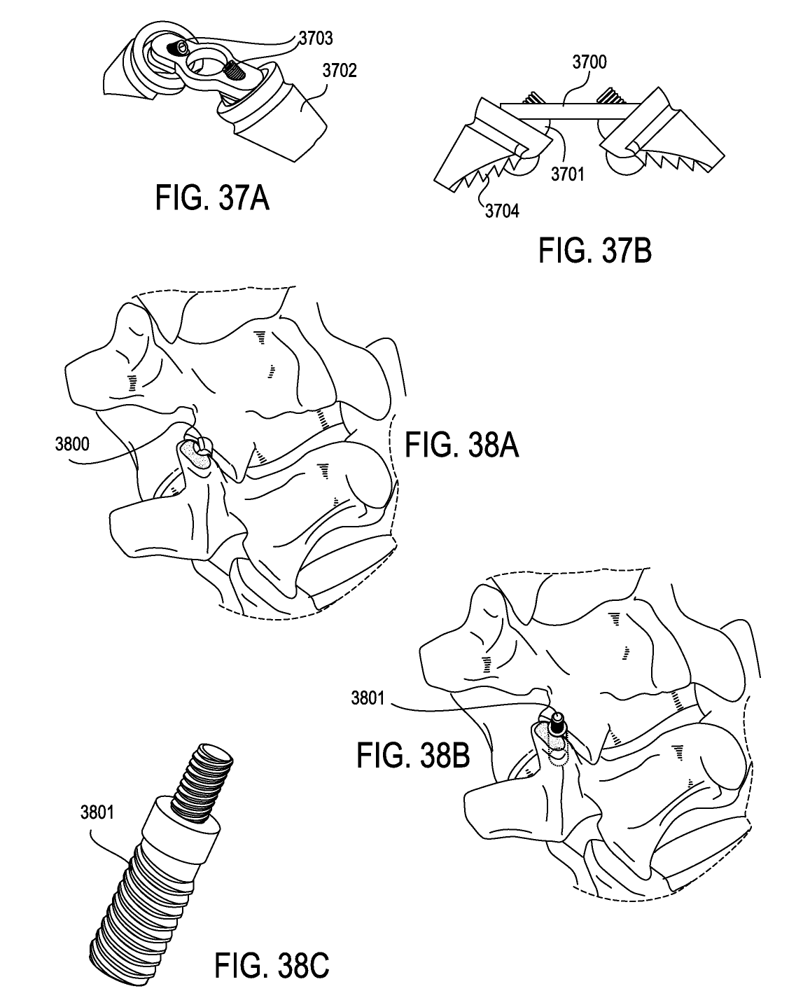

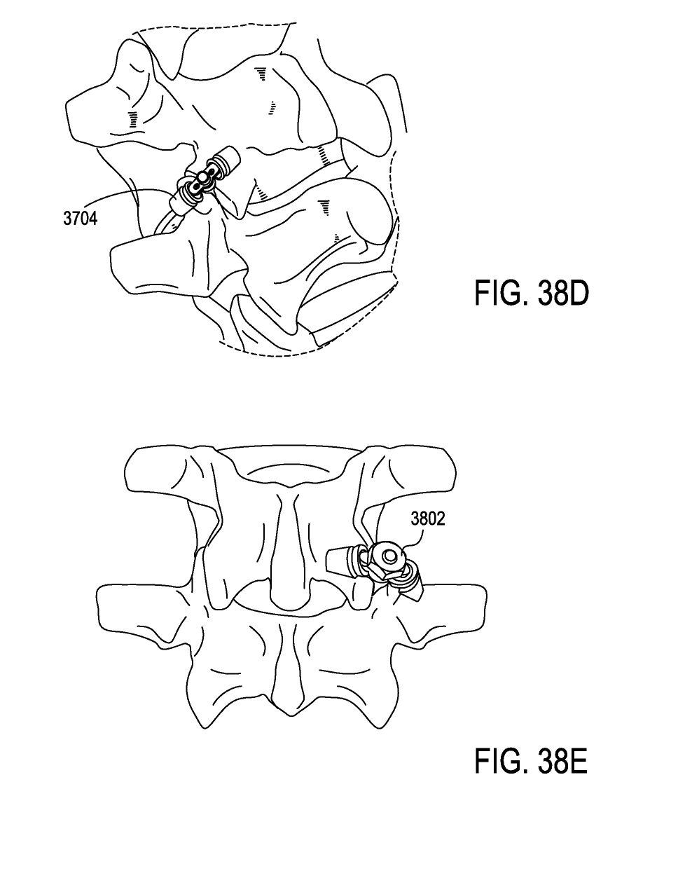

FIGS. 37A to 38E show an exemplary embodiment of a facet fixation system. The fixation device and system described in reference to FIGS. 37A to 38E may be similar to the device described in reference to FIGS. 34-36D with alternative variations of the facet cap. In this variation, the facet cap may be an articulating cap that comprises a plate 3700, a joint 3701, and wing 3702. As shown in FIGS. 37A and 37B, the facet cap in this embodiment includes a first wing that couples to a first portion of the facet joint and a second wing that couples to a second portion of the facet joint. The first and second wings are coupled to a plate of the facet cap via a joint. The joint may be a ball joint, a gimbal joint, or any other suitable joint. The facet cap may further include a set screw 3703. The set screw may function to lock the position of the wings with respect to the plate. In some embodiments, the wings may include coupling members 3704 such as teeth. Alternatively, the coupling members may be spikes or protrusions sized and configured to dig into or catch onto bone thereby aiding in the coupling of the wing to the facet joint. Alternatively, the coupling members may be an abrasive surface, abrasive particles, or may include material such as bone cement or bone graft (including BMP for example). FIGS. 38A-38E illustrate a method of implantation for the facet fixation device described in reference to FIGS. 37A and 37B. This method may be similar to the method described in reference to FIGS. 30A-30D. As shown in FIG. 38A, a hole 3800 may be created within a facet joint. The hole may be drilled, punched, broached, etc. As shown in FIG. 38B, a fixation screw 3801 may be positioned (e.g. threaded) into the hole. As shown in FIG. 38D, the facet cap may be placed over the fixation screw and the facet joint. A tool may be used to move the adjustable wings onto/into the facet joint. Once the wings are in position, a set screw may be used to fix the wings in place. As shown in FIG. 38E a nut 3802 or other suitable fastener may be coupled to the end of the fixation screw thereby securing the plate of the facet cap to the fixation screw.

### Unilateral Interspinous Fusion

As mentioned above, described herein are devices, systems and methods for performing an interspinous fusion unilaterally. In many variations, the devices and systems described herein are configured to be deployable from a single side of the spine (i e unilaterally deployable). In some embodiments, the devices and systems may be configured to be deployable to a single side of the spine through a single incision, e.g. a midline incision or a paramedian incision. Also described herein are facet fixation devices and methods. In some embodiments, the fixation device includes a fixation screw configured to be implanted into a facet joint and a facet cap configured to couple to the fixation screw and to couple to a medial aspect and a lateral aspect of a facet joint.

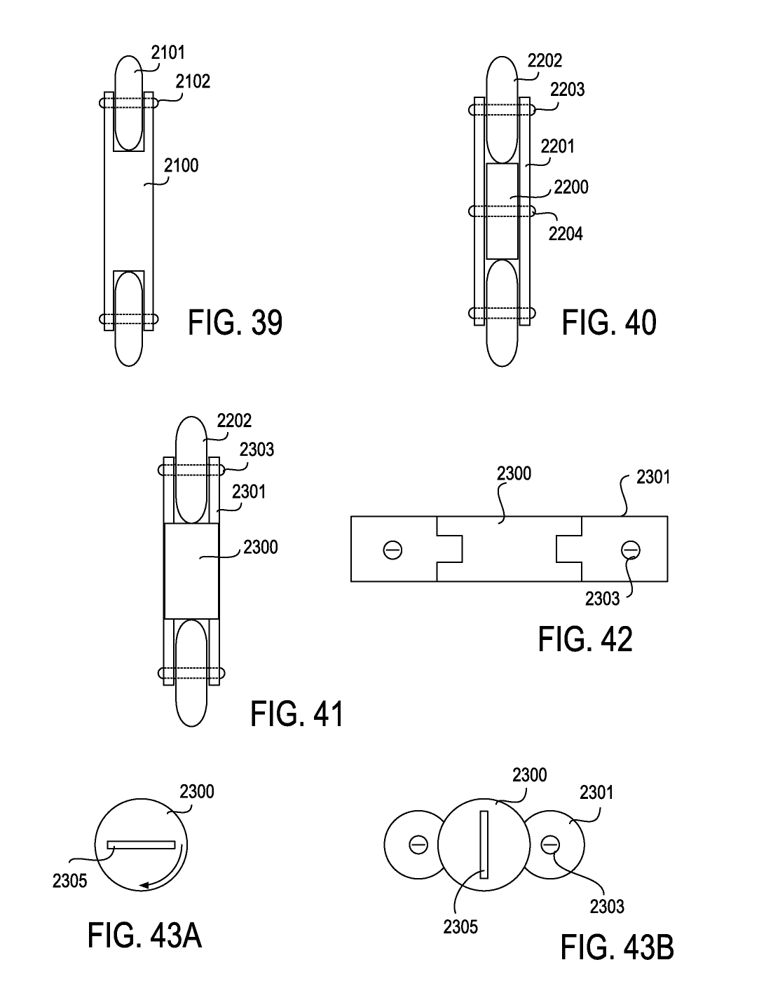

For example, described herein are methods for performing spinal fusion by way of a spinous process fixation system, such as the one shown in FIG. 39, which couples to two adjacent spinous processes 2101. The device 2100 (which may be used as part of a system) is coupled to each of the spinous processes via a screw 2102 or other suitable fixation element such as a pin. The device 2100 may function to fix a portion of the spine in place and may also distract the two spinous processes. As shown in FIG. 40, the Spinous process fixation system comprises a center distraction portion 2200 and two coupling portions 2201. The distraction portion 2200 may function to distract the two spinous processes 2202. The coupling portion 2201 may function to couple to the two spinous processes 2202 via screws 2203. In order to deliver the Spinous process fixation system, in some embodiments, the center portion 2200 may first be delivered between the spinous processes. In some embodiments, the center portion 2200 may be positioned via pull wire as described above. Once portion 2200 is in place, a first coupling portion 2201 may be delivered laterally from a first side, and then the second coupling portion may be delivered laterally from a second side. The coupling portions may then be coupled to the spinous processes via screws 2203. In some variations, the coupling portions may be coupled to the center portion via screw 2204.

As shown in FIG. 41, a spinous process fixation system/device may include a center distraction portion 2300 and four coupling portions 2301. The distraction portion 2300 may function to distract the two spinous processes 2302. The coupling portion 2301 may function to couple to the two spinous processes 2302 via screws 2303. In order to deliver the spinous process fixation system, in some embodiments, the center portion 2200 may first be delivered between the spinous processes. In some embodiments, the center portion 2200 may be positioned via pull wire as described above. Once portion 2300 is in place, a first coupling portion 2301 may be delivered laterally from a first side to a first spinous process 2302 and then a second coupling portion may be delivered laterally to the first spinous process 2302 from a second side. Next, a third coupling portion may be delivered laterally from a first side to a second adjacent spinous process and then a fourth coupling portion may be delivered laterally to the second spinous process from a second side. The coupling portions may then be coupled to the spinous processes via screws 2203. In some embodiments, each of the coupling portions may be delivered via pullwire. As shown in FIG. 42, the coupling portions 2301 may be configured such that they are slidably connected to the center portion 2300. As shown in FIGS. 43A and 43B, the coupling portions may be configured such that they are stowed within the center portion 2300, as shown in FIG. 43A, while the device is delivered. Once the device is in place, the coupling portions 2301 may be released such that they extend from the center portion 2300, as shown in FIG. 43B. In some embodiments, the coupling portions 2301 may be rotated out of the center portion 2300. For example, a user may couple a tool to slot 2305 and rotate the slot such that the coupling portions are rotated out from the center portion.

As mentioned, described herein are devices, systems and method for performing spinal fusion surgery, in particular a less invasive posterior column fusion or soft fusion, by way of an interspinous fusion or spinous process fixation, for example. Additionally, facet joint fixation may also be performed—either bilaterally or unilaterally. In some embodiments, a spinal decompression procedure may also be performed, since it may enhance success rates of the spinal fusion procedure.

These systems and methods may be particularly useful for percutaneous treatments of one or more body region. However, it should be understood than any of the devices, methods and systems described herein may be used as part of an “open” surgical procedure in which access to a body region is created through an opening in the tissue (e.g., by movement or removal of tissue). Any of the systems and devices described may be performed as part of a procedure that is at least partially open. Partially percutaneous procedures may also be performed using these devices, systems and methods.

Described herein are devices, systems and methods for performing an interspinous fusion unilaterally. The devices and systems described herein are configured to be deployable from a single side of the spine (i e unilaterally deployable). In some embodiments, the devices and systems may be configured to be deployable to a single side of the spine through a single incision, e.g. a midline incision or a paramedian incision.

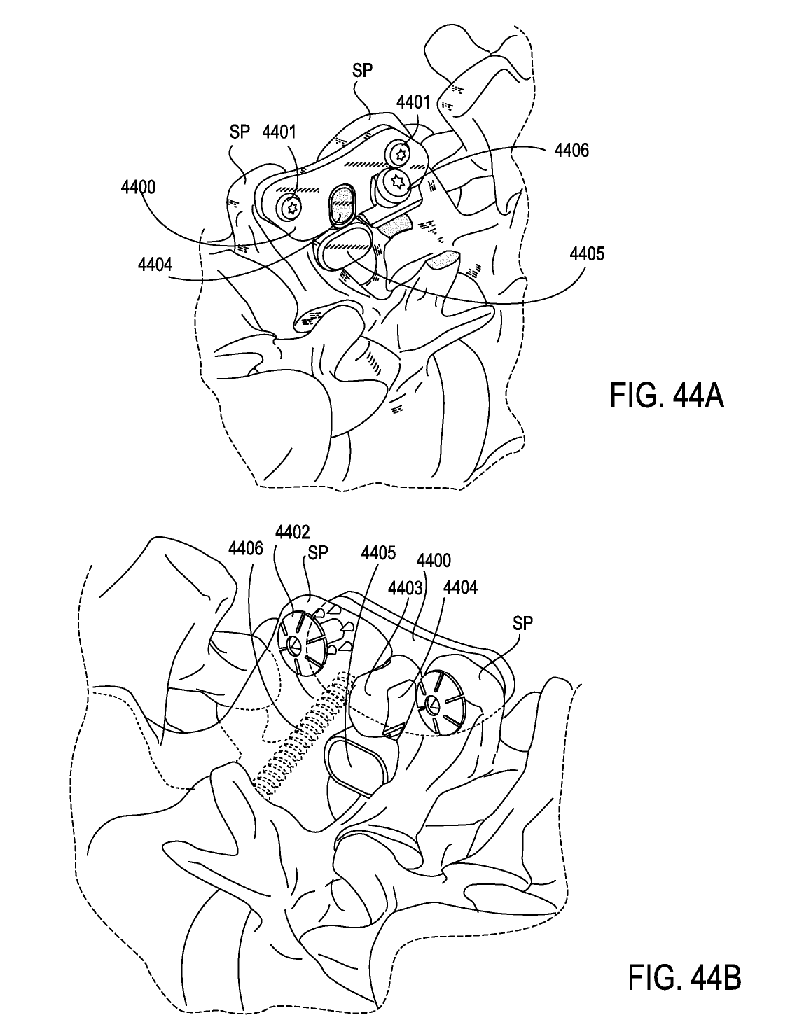

FIGS. 44A and 44B show an exemplary embodiment of an interspinous fusion device, specifically one that may be deployable unilaterally. As shown, the devices and systems may include a fixation plate 4400; a coupling screw 4401, sized and configured to couple the fixation plate to a fist lateral side of a spinous process (SP); and a coupling backing 4402, sized and configured to couple to the opposite lateral side of the spinous process. As shown, the fixation plate may be positioned within the spine such that it is coupled to two adjacent spinous processes. The fixation plate may be positioned on a single lateral side of the spinous processes. The fixation plate may be attached to both spinous processes to stabilize the segment of the spine and promote fusion. The fixation plate may be coupled to the spinous processes by way of coupling screws for example. The screws may be fed through holes in the fixation plate and then into each of the spinous processes. The screws may be deployed into the fixation plate (and into the spine) from the single lateral side of the spinous processes—e.g. through the same incision as the fixation plate. In some alternative variations, the screws may be dowels or bone dowels, for example. The fixation plate may be coupled to the spinous processes in any suitable location. For example, the fixation plate may be couple to the mid to posterior portion of the spinous processes, as shown in FIGS. 44A and 44B. Alternatively, the fixation plate may be placed more ventrally (anterior), near the lamina, rather than mid-high on the spinous process. For example, although not shown, the fixation plate may be placed in the more anterior position such that the post may be positioned about where the spacer is located in FIG. 44A, thereby replacing the spacer.

In some embodiments, as shown in FIG. 44B, the fixation plate may further include a post 4403. The post may be coupled to the back side of the fixation plate (i.e. the side facing the spinous processes while implanted) and may be sized and configured to be positioned between two adjacent spinous processes. In some embodiments, the post may further function to distract the two spinous processes. As shown in FIGS. 44A and 44B, the post may also be sized and configured to receive a graft material 4404. The graft material may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the post may be filled with a filer material such as bone cement. As shown in FIG. 44B, the post is configured such that is has a window or windows through which, the bone graft may contact the spinous processes and, in some instances, promote fusion. In some embodiments, the spinous processes may be decorticated prior to the implantation of the fixation plate and/or bone graft to also, in some instances, promote fusion.

In some embodiments, as shown in FIGS. 44A and 44B, the devices and systems described herein may further include a spacer 4405. The spacer may be metallic or plastic. For example, the spacer may be made out of polyetheretherketone (PEEK). Alternatively, the spacer may be a bone graft material as described above. The spacer may be implanted between the two spinous processes. In some embodiments, the spacer may be placed from the same lateral side of the spinous processes as the fixation plate and/or bone graft material. The spacer may function to distract the spinous processes, thereby distracting areas that are pressing on the spinal cord and/or nerves; promote fusion between the spinous processes to provide long-term spine stabilization; and/or provide a protective cover for the spinal cord to help prevent scar tissue from pressing on the spinal cord and/or nerves. The spacer may be placed in the spine before or after the fixation plate is placed within the spine. In some embodiments, the spacer may be removed once the fixation plate is connected to the spinous process(es). As shown, the spacer may be positioned anterior to the fixation plate. This may be advantageous due to the fact that there may be more substantial bone surfaces for the spacer to contact in this area, furthermore, these portions of the spinous processes may be stronger and more stable to receive forces from the spacer. Alternatively, the spacer may be positioned posterior to the fixation plate. In some alternative embodiments, the spacer may replace the post of the fixation plate.

As shown in FIGS. 44A and 44B, the fixation system in this embodiment may include a fixation screw 4406. For example, as shown, the fixation screw may be a contralateral trans-laminar facet screw. As shown, the trans-laminar facet screw may be deployed from the first lateral side of the spinous process through the fixation plate, into the fist lateral side of a spinous process, and into a facet joint on the opposite (contralateral) side of the spinous process from the fixation plate. This is shown in FIG. 44B, which shows the cephalad (left in this figure) vertebra to be translucent. FIG. 44B also shows the coupling backing, sized and configured to couple to the opposite lateral side of the spinous process. In some embodiments, the coupling backing is the distal end of the coupling screw that is configured to expand or flare out and couple to the opposite lateral side of the spinous process upon exiting from the spinous process. Alternatively, the coupling backing may be implanted through the incision and coupled to the distal end of the coupling screws after the screws have been deployed through the spinous processes. In some alternative embodiments, the fixation system or device may not include a coupling backing. For example, the coupling screw may be a dowel, such as a bone dowel. Alternatively, the screw may be an expanding or otherwise self securing screw that may be fixed to the spinous processes without a coupling backing.

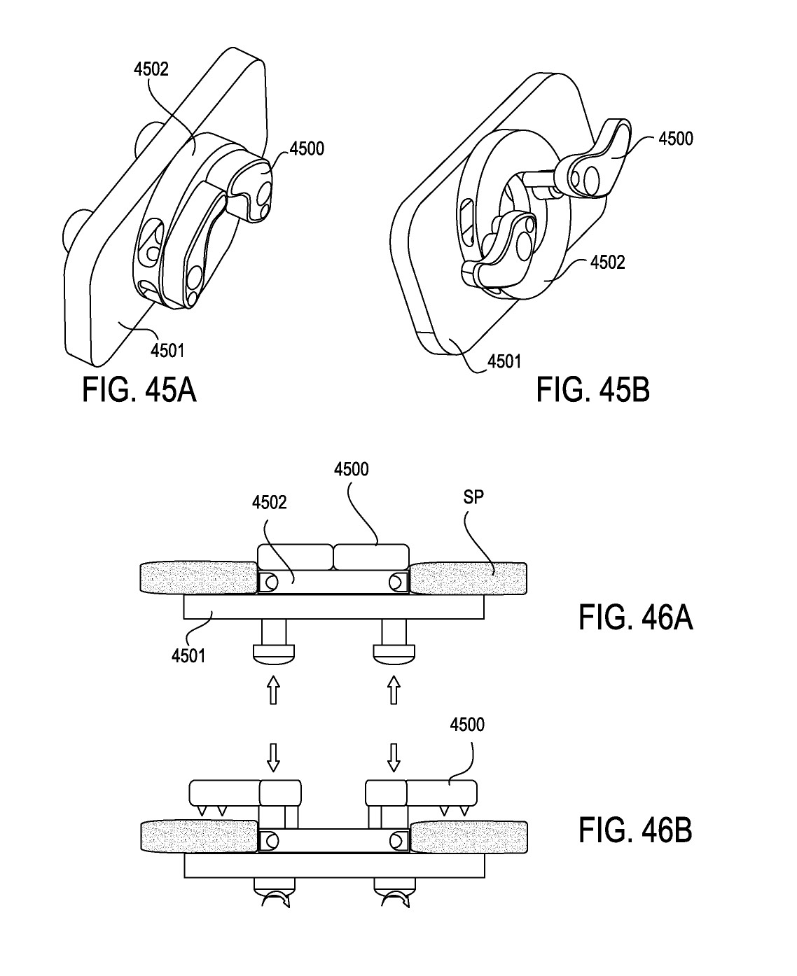

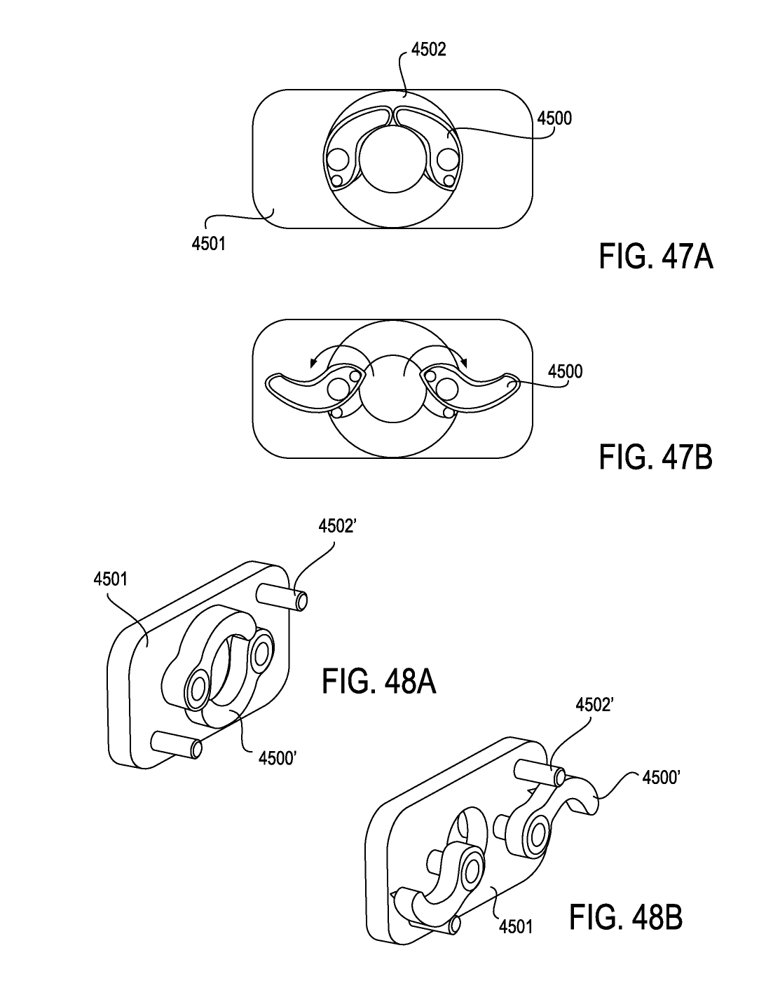

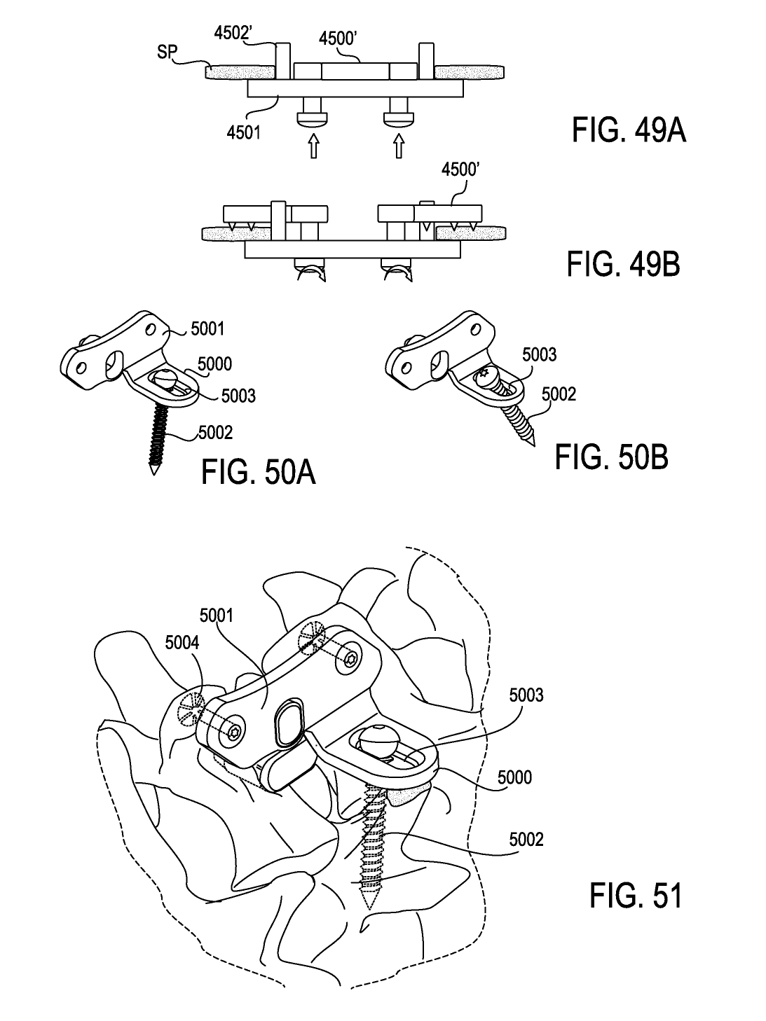

FIGS. 45A to 49B show various exemplary embodiments of an interspinous fusion device having alternative embodiments of deployable coupling backings. FIGS. 45A, 45B, 48A, and 48B show isometric views of the device. FIGS. 46A, 46B, 49A, and 49B show top views of the device, looking down at the spine—posterior to anterior. FIGS. 47A and 47B illustrate side views of the device. As shown, these coupling backings 4500 may be coupled to the fixation plate 4501 and may be sized and configured to couple to the opposite lateral side of the spinous process (SP). The coupling backing may be sized and configured to operate in one of three modes: a non-deployed mode, wherein the coupling backing fits between two adjacent spinous processes (as shown in FIGS. 45A, 46A, 47A, 48A, and 49A); a deployed mode, wherein the coupling backing rotates behind a spinous process (as shown in FIGS. 45B, 46B, 47B, 48B, and 49B); and a compression mode, wherein the coupling backing is compressed against the opposite lateral side of the spinous process (as shown in FIGS. 45B, 46B, 47B, 48B, and 49B). The spinous process fixation devices in these embodiments may further include a coupling screw(s). As shown in FIGS. 45B, 46B, 47B, 48B, and 49B, the coupling backing may transition from the non-deployed mode (FIGS. 45A, 46A, 47A, 48A, and 49A) to the deployed mode and from the deployed mode to the compression mode by rotating the coupling screw. The coupling screw may be coupled to the coupling backing such that as the screw is rotated, it rotates the arms of the coupling backing. The arms will be rotated by the screw until they reach a certain position. At that point, the rotation of the screw will begin to advance the arms of the coupling backing toward the fixation plate and the spinous processes. The more the screws are rotated the more the arms of the coupling backing will advance, thereby placing the coupling backing and the fixation plate in compression about the spinous processes. As shown in FIGS. 48A and 48B, the arms of the coupling backing will rotated until they reach the post 4502′. Once they reach the post, the post will prevent further rotation, and the rotation of the screw will advance the arms toward the fixation plate. In some embodiments, as shown in FIGS. 46A, 46B, 49A, and 49B, the coupling screws may also advance the arms of the coupling backing further between the spinous processes.

FIGS. 50A to 51 show an alternative exemplary embodiment of an interspinous fusion device. This device may be similar to the device described in reference to FIGS. 44A and 44B with the addition a bracket 5000 coupled to the front side of the fixation plate 5001 (i.e. the side facing away from the spinous processes while implanted). As shown, the bracket may be at an angle to the front face of the fixation plate. For example, as shown, the bracket may be about 90 degrees from the fixation plate such that the bracket and fixation plate are “L-shaped”. Alternatively, the bracket may be coupled to the fixation plate at any suitable angle. As shown in FIGS. 50A to 51, the fixation system in this embodiment may include a fixation screw 5002. The bracket may define a slot 5003 through which the fixation screw may be implanted. The slot may be sized and configured to receive the fixation screw at any suitable angle (compare FIGS. 50A and 50B) and at any suitable distance from the fixation plate. This slot may therefore allow the same fixation device and system to be used on patients with varying anatomy, for example the distance from the lateral side of the spinous process to facet joint may vary from spine level to spine level within a single spine and from patient to patient. As shown in FIG. 51, the system may include an ipsilateral trans-facet interpedicular screw. Alternatively, it may include a trans-facet screw, a pedicle screw, a facet dowel, or any other suitable fixation device, or combination thereof. As shown, the trans-facet interpedicular screw may be deployed through the slot of the bracket and into a facet joint on the same (ipsilateral) side of the spinous process as the fixation plate. FIG. 51 also shows the coupling backing 5004 coupled to the caudal spinous process. In some embodiments, instead of or in addition to a fixation screw, a facet dowel and/or a facet fixation strap may be used. In some embodiments, the bracket may be sized and configured to receive and/or secure a facet fixation strap around the facet joint. The facet fixation strap may be used instead of or in addition to a contralateral fixation screw and/or a ipsilateral fixation screw.

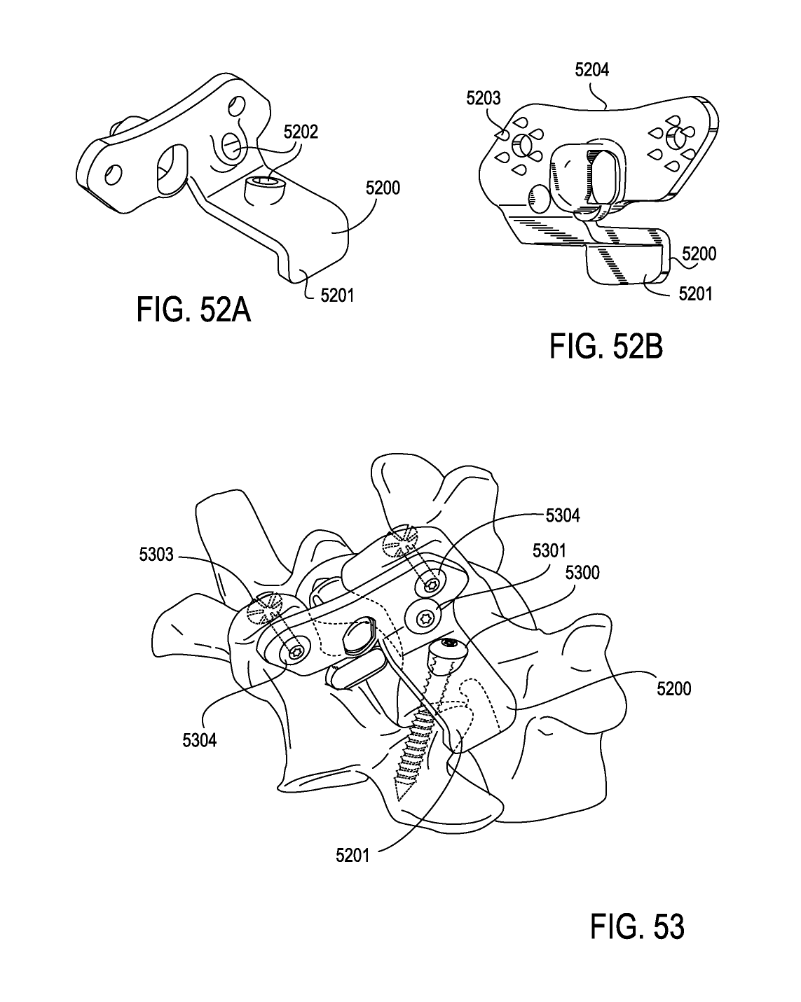

FIGS. 52A to 54 show an exemplary embodiment of an interspinous fusion device, specifically one that may be deployable unilaterally. This device may be similar to the device described in reference to FIGS. 50A to 51 with an alternative variation of the bracket 5200 coupled to the front side of the fixation plate (i.e. the side facing away from the spinous processes while implanted). In this variation, the bracket includes an lip portion 5201 that is sized and configured to wrap around a portion of the facet joint (FJ). In some embodiments, the bracket width may be adjustable such that it may be adjusted to fit various anatomical sizes and structures.

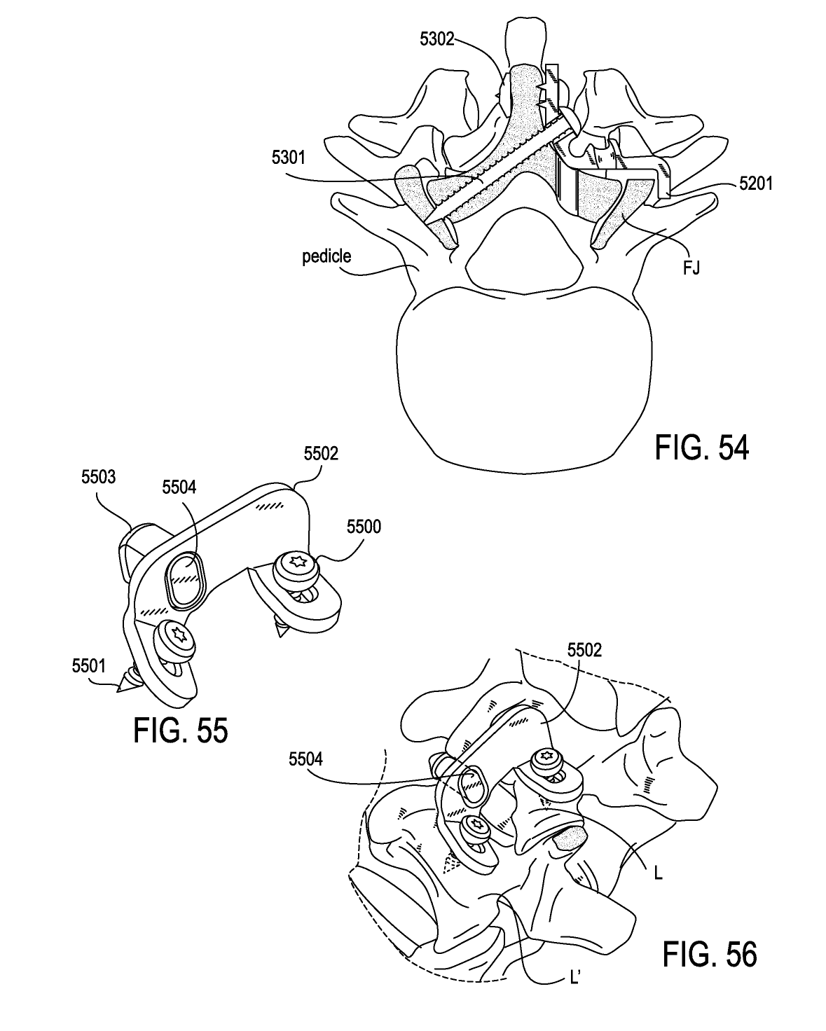

Also in this embodiment, as shown in FIG. 53, the fixation device may include both an ipsilateral fixation screw 5300 and a contralateral fixation screw 5301. For example, the fixation device or system may include both a contralateral trans-laminar facet screw in addition to an ipsilateral trans-facet interpedicular screw. Alternatively, the ipsilateral fixation device may be an ipsilateral trans-facet screw, a pedicle screw, a facet dowel, or any other suitable fixation device deployable ipsilaterally or combination thereof. Referring back to FIGS. 52A and 52B, the bracket may define a hole or slot 5202 through which the fixation screws may be implanted. The hole may be sized and configured to receive the fixation screw at any suitable angle and at any suitable distance from the fixation plate. For example, as shown in cross section in FIG. 54, the fixation screw hole defined by the fixation plate may be angled to receive and position a contralateral fixation screw 5301. For example, as shown, the contralateral fixation screw may be a contralateral trans-laminar facet screw. As shown in cross section in FIG. 54, the trans-laminar facet screw may be deployed from the first lateral side of the spinous process through the fixation plate, into the fist lateral side of a spinous process, and into a facet joint on the opposite (contralateral) side of the spinous process from the fixation plate. The fixation screw hole defined by the bracket, as shown in FIGS. 54 and 52A, may be angled to receive and position an ipsilateral fixation screw. As shown in FIG. 53, the system may include an ipsilateral trans-facet interpedicular screw. Alternatively, it may include a trans-facet screw, a pedicle screw, a facet dowel, or any other suitable fixation device. As shown, the trans-facet interpedicular screw may be deployed through the slot of the bracket and into a facet joint on the same (ipsilateral) side of the spinous process as the fixation plate. FIG. 53 also shows the coupling backing 5303 coupled to the caudal spinous process.

Also shown in FIG. 52B are coupling members 5203 positioned on the back side of the fixation plate 5204 (i.e. the side facing the spinous processes while implanted). Coupling members may be incorporated into any of the various embodiments, and variations thereof, described herein. For example, as shown, the coupling members may be spikes or protrusions sized and configured to dig into or catch onto bone thereby aiding in the coupling of the fixation plate to the spinous process(es). Alternatively, the coupling members may be an abrasive surface, abrasive particles, or may include material such as bone cement or bone graft (including BMP for example). In some embodiments, the anterior side of the bracket may also include coupling members.

An exemplary method for performing an interspinous fusion unilaterally (i.e. through a single incision and/or from a single side of the spine) is also described herein. As described throughout, the devices and systems may be configured to be deployable to a single side of the spine through a single incision, e.g. a midline incision or a paramedian incision. In this example, the method may also include the optional step of performing a decompression, as shown in FIG. 30B. FIGS. 30A-30C show the steps of preparing a portion of the spine for fusion. In some embodiments, some or all of these steps may be optional. In some embodiments, as shown in FIG. 30A, a laminotomy may be performed. This step includes removing bone from the lamina of a vertebra. The laminotomy may be performed using a Rongeur, such as a Karrison Rongeur, or may alternatively be performed using any other suitable bone cutting, drilling, or biting device. In some embodiments, as shown in FIG. 30B, a decompression may be performed. For example, a tissue modification device (not shown) may fed around the facet joint by a pullwire through an interlaminar window, around the facet joint, and out through a spinal foramen. Once in position, the tissue modification device may be bimanually reciprocated against the tissue of the facet joint to modify and/or remove tissue. The tissue modification device may perform a decompression and/or may create a trough around the outer surface of the facet joint (as shown in FIG. 30B) and prepare the outer surface of the facet joint to receive a facet fixation strap. Using a probe access system and a pullwire deployment system as described in the references listed above and incorporated by reference in their entirety, a decompression may be performed on the ipsilateral side, on the contralateral side, or both. Once a decompression is performed on the contralateral side, a contralateral facet fixation strap as described in the references listed above and incorporated by reference in their entirety, may be placed around the contralateral facet joint. This facet fixation strap may be used in lieu of or in addition to a contralateral fixation screw.

In some embodiments, as shown in FIG. 30C, the posterior surface of the joint may be prepared to receive a bracket portion of the fixation device. Specifically, as shown, portions of the superior articular process (SAP) may be removed to shape and prepare the surface of the joint to receive the bracket portion of the fixation device. In some embodiments, additionally or alternatively, the inferior articular process (IAP) may be modified and/or prepared. In some embodiments, the articulating surfaces of the joint may be roughened or otherwise modified to promote fusions of the surfaces. For example, the outer cancellous bone or the articulating cartilage of the joint may be removed. By removing this bone from each articulating surface and thereby approximating a bloody bone surface to bloody bone surface, fusion is more likely to occur. Additionally bone cement or bone paste (including BMPs) may be placed within the joint. Additionally, in some embodiments, the surfaces of the spinous processes may be roughened or otherwise modified to promote fusions of the surfaces. The surfaces that may be roughened may include the outer lateral surfaces where the fixation plate is coupled and or the inner surfaces facing the post of the fixation device and/or the spacer. For example, the outer cortical bone may be removed e.g. decorticated. By removing this bone from each surface and thereby approximating a bloody bone surface to bloody bone surface via a bone graft material (in the spacer or post of the fixation device, for example), fusion is more likely to occur. Additionally bone cement or bone paste (including BMPs) may be placed on a surface of the spinous process(es).

As shown in FIG. 53, once areas of the spine have been adequately prepared, the fixation device may be implanted. In this example, a fixation plate having a bracket 5200 with a lip 5201 is implanted from the first lateral side of the spine. As shown, two coupling screws 5304 may be placed into the fixation plate from the same lateral side of the spine. The coupling screws may fix the fixation plate to the spinous process(es) and/or a coupling backing may be implanted. A graft material may also be implanted with the fixation plate. The graft may be inserted into the fixation plate prior to implantation or may be positioned once the fixation plate is positioned and/or secured to the spinous processes. Additionally, a spacer may be implanted as described above. In some embodiments, the spacer may be implanted and may distract the two adjacent spinous processes prior to the implantation of the fixation plate. As shown in FIG. 53, the fixation screws are implanted as described above. In this embodiment, the fixation system may include both a contralateral trans-laminar facet screw 5301 and an ipsilateral trans-facet interpedicular screw 5300 or an ipsilateral trans-facet screw. Alternatively, the ipsilateral fixation device may be a pedicle screw, a facet dowel, or any other suitable fixation device. FIG. 53 illustrates the implantation of the contralateral trans-laminar facet screw from the first lateral side of the spinous process through the fixation plate, into the fist lateral side of a spinous process, and into a facet joint on the opposite (contralateral) side of the spinous process from the fixation plate. As described above, an ipsilateral fixation screw may also be implanted through the fixation plate (not shown). Alternatively, a facet dowel and/or a facet fixation strap may be used to fix and/or fuse the ipsilateral joint.

FIGS. 55 to 57B show an exemplary method for performing fusion unilaterally. In this example, the fixation device may be sized and configured to be used in a method for performing an interlaminar fusion unilaterally. As shown, this fixation device includes a bracket 5500 that is sized and configured to be coupled to a first lamina (L) of the spine via a coupling screw. The first lamina is located on a first lateral side of the spinous process. In some embodiments, the fixation device includes a second bracket 5501 that is sized and configured to be coupled to a second, adjacent lamina (L′) of the spine via a second coupling screw. The second lamina is located on the same first lateral side of the spinous process as the first adjacent lamina. The fixation device may be attached to both laminas, thereby connecting two adjacent vertebra, to stabilize the segment of the spine and promote fusion.

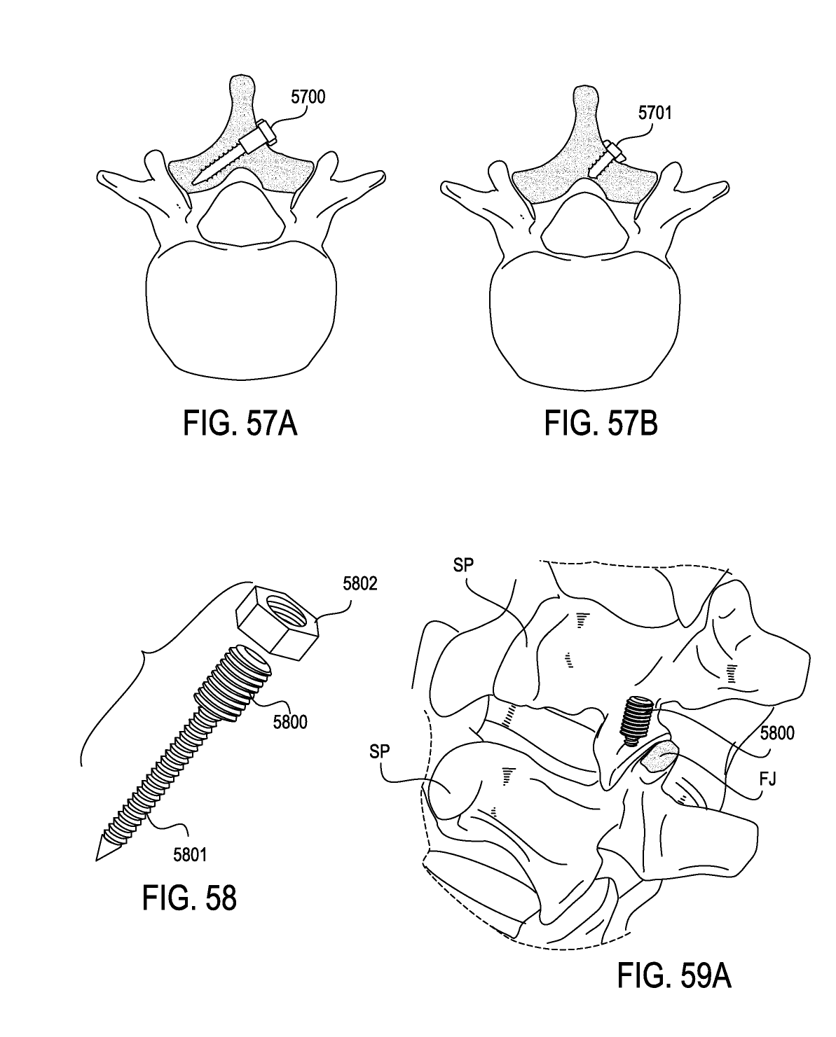

As shown, the fixation device may also include a fixation plate 5502 that is sized and configured to couple the first bracket to the second bracket. As shown, the fixation plate may be positioned within the spine such that it is adjacent and in some cases coupled to two adjacent spinous processes. The fixation plate may be positioned on a single lateral side of the spinous processes, i.e. the same side as the brackets. The fixation plate may be attached to both spinous processes to stabilize the segment of the spine and promote fusion. The fixation plate may be coupled to the spinous processes by way of coupling screws for example. Alternatively, the fixation plate may be directly coupled to the brackets (not shown) and may not be positioned adjacent to the spinous processes. As shown in FIGS. 57A and 57B, the coupling screw 5700 or 5701 may couple the fixation plate through the lamina contralaterally. As shown, any suitable size and configuration of screw may be used and it may be deployed at any suitable angle. The screw may be deployed such that it does not damage vascular or neural tissue such as the spinal cord or cauda equina.

In some embodiments, as shown in FIGS. 55A to 55C, the fixation device may further include a post 5503 or barrel. The post may be coupled to the back side of the fixation plate (i.e. the side facing the spinous processes while implanted) and/or may be coupled directly to one or more of the brackets. The post may be sized and configured to be positioned between two adjacent spinous processes. In some embodiments, the post may be positioned toward the anterior portion of the spinous process and/or between two adjacent laminas. This may be advantageous due to the fact that there may be more substantial bone surfaces for the post to contact in this area, furthermore, these portions of the spinous processes/lamina may be stronger and more stable to receive forces from the post. In some embodiments, the post may receive the majority of the loads from the spinous processes. In some embodiments, the post of the fixation plate may be placed more ventrally (anterior), near the lamina, rather than mid to high between the spinous processes. For example, although not shown, the post of the fixation plate may be placed in the more anterior position where the spacer is located in FIG. 44A, thereby replacing the spacer.

In some embodiments, the post may further function to distract the two spinous processes. As shown in FIGS. 55A and 56, the post may also be sized and configured to receive a graft material 5504. The graft material may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the post may be filled with a filer material such as bone cement. As shown, the post is configured such that is has a window or windows through which, the bone graft may contact the spinous processes and/or lamina and, in some instances, promote fusion. In some embodiments, the spinous processes and/or lamina may be decorticated prior to the implantation of the fixation plate and/or bone graft to also, in some instances, promote fusion.

In some embodiments, the devices and systems described herein may further include a spacer rather than (or in addition to) the post (described above). The spacer may be metallic or plastic. For example, the spacer may be made out of polyetheretherketone (PEEK). Alternatively, the spacer may be a bone graft material as described above. The spacer may be implanted between the two spinous processes and/or lamina. In some embodiments, the spacer may be placed from the same lateral side of the spinous processes as the fixation plate and/or bone graft material. The spacer may function to distract the spinous processes, thereby distracting areas that are pressing on the spinal cord and/or nerves; promote fusion between the spinous processes to provide long-term spine stabilization; and/or provide a protective cover for the spinal cord to help prevent scar tissue from pressing on the spinal cord and/or nerves. The spacer may be placed in the spine before or after the fixation plate is placed within the spine. In some embodiments, the spacer may be removed once the fixation plate is connected to the spinous process(es).

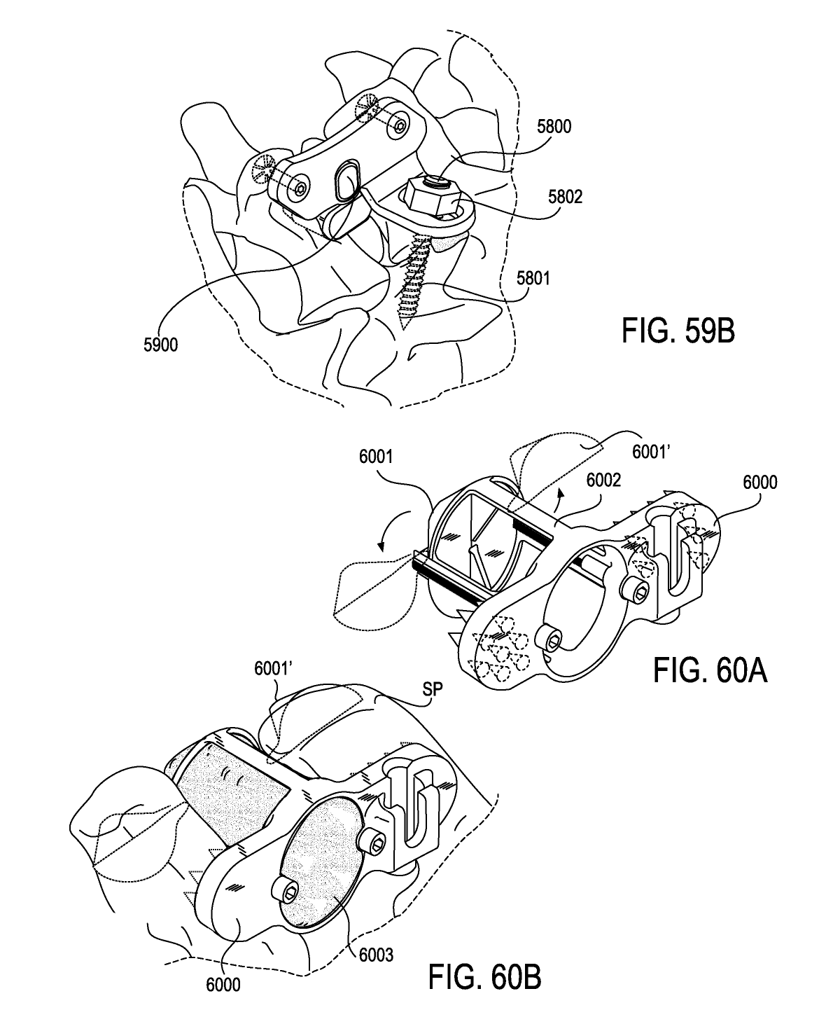

FIGS. 59A and 59B, show an exemplary method for performing an interspinous fusion unilaterally (i.e. through a single incision and/or from a single side of the spine). The fixation device and system described in reference to FIGS. 58 to 59B may be similar to the device described in reference to FIGS. 50A to 51 with an alternative variation of the bracket coupled to the front side of the fixation plate (i.e. the side facing away from the spinous processes while implanted). In this variation, the bracket includes an alternative slot that is sized and configured to be placed over a fixation screw head 5800 that may be implanted into the spine.

As shown in FIG. 58, the fixation screw 5801 of this embodiment includes a fixation screw head 5800. The screw head is sized and configured to receive a coupling member, such as a fixation nut 5802 (FIG. 58) for example. As shown, the screw head may be threaded or may alternatively receive a coupling member in any other suitable variation. FIG. 59A illustrates the fixation screw implanted within the spine. In some embodiments, the fixation screw may be a trans-facet interpedicular screw. Alternatively, the fixation screw may be a trans-facet screw, a pedicle screw, a facet dowel, or any other suitable fixation device. As shown, the trans-facet interpedicular screw may be implanted into a facet joint (FJ) and then into a pedicle (not shown) on a first lateral side of the spinous process. In some embodiments, FIG. 59A illustrates the first step of an alternative method for performing an interspinous fusion unilaterally. As shown in FIG. 59B, once the fixation screw is implanted into the spine, a fixation device may be placed over the fixation screw and into position from the same first lateral side of the spinous process. As shown, the slot of the bracket is sized and configured to be placed over the head of the fixation screw. In some embodiments, the slot may larger than the fixation screw head such that the fixation plate may be moved and positioned even after it is placed over the fixation screw head.

As shown in FIG. 59B, once the fixation plate is implanted the graft material 5900, the fixation nut 5802, and the coupling screws may be implanted. The fixation nut and the coupling screws may be coupled loosely to the fixation screw and the fixation plate, respectively, and then they may all be tightened down once the fixation plate and bracket are properly positioned. Alternatively, the fixation screw, the fixation plate, the graft, and the coupling screws may be placed and/or fixed into position in any suitable order. In some embodiments, the slot and the fixation nut may be replaced by any alternative coupling mechanism sized and configured to couple the fixation plate to the fixation screw head. For example, the bracket may include an integrated fastener.

In some alternative embodiments, the fixation plate may not include a bracket portion. In this embodiment, the fixation screw (such as a trans-facet interpedicular screw, a trans-facet screw, or a pedicle screw) may be implanted as shown in FIG. 59A and the fixation plate (without a bracket) may also be implanted as described herein (see FIG. 44A, for example). In this embodiment, the fixation plate may be implanted before the fixation screw or vice versa. Once the fixation screw and the fixation plate are implanted (from the same lateral side of the spinous processes), a connecting element may be implanted to connect the fixation screw to the fixation plate. The connecting element may be a rigid or flexible rod for example. The connecting element may be provided in one of several different sizes. This connecting element may therefore allow the same fixation device and/or system to be used on patients with varying anatomy, for example the distance from the lateral side of the spinous process to facet joint may vary from spine level to spine level within a single spine and from patient to patient. Alternatively, the connecting element may be adjustable. It may be adjusted prior to or following implantation.

FIGS. 60A and 60B show an alternative exemplary embodiment of an interspinous fusion device. This device may be similar to the device described in reference to FIGS. 45A to 49B. As described above, the interspinous fusion device of this embodiment may include a fixation plate 6000 sized and configured to couple to a first lateral side of a spinous process and a coupling backing 6001, coupled to the fixation plate and sized and configured to couple to the opposite lateral side of the spinous process. As described above, the coupling backing is sized and configured to operate in one of two modes: A non-deployed mode, labeled 6001 in FIG. 60A, in which the coupling backing fits between two adjacent spinous processes such that the device may be placed and deployed unilaterally; and a deployed mode, labeled 6001′ in FIG. 60A, in which the coupling backing rotates behind a spinous process (SP) (FIG. 60B). As described above, the device may further include a post 6002 and/or a graft material 6003.

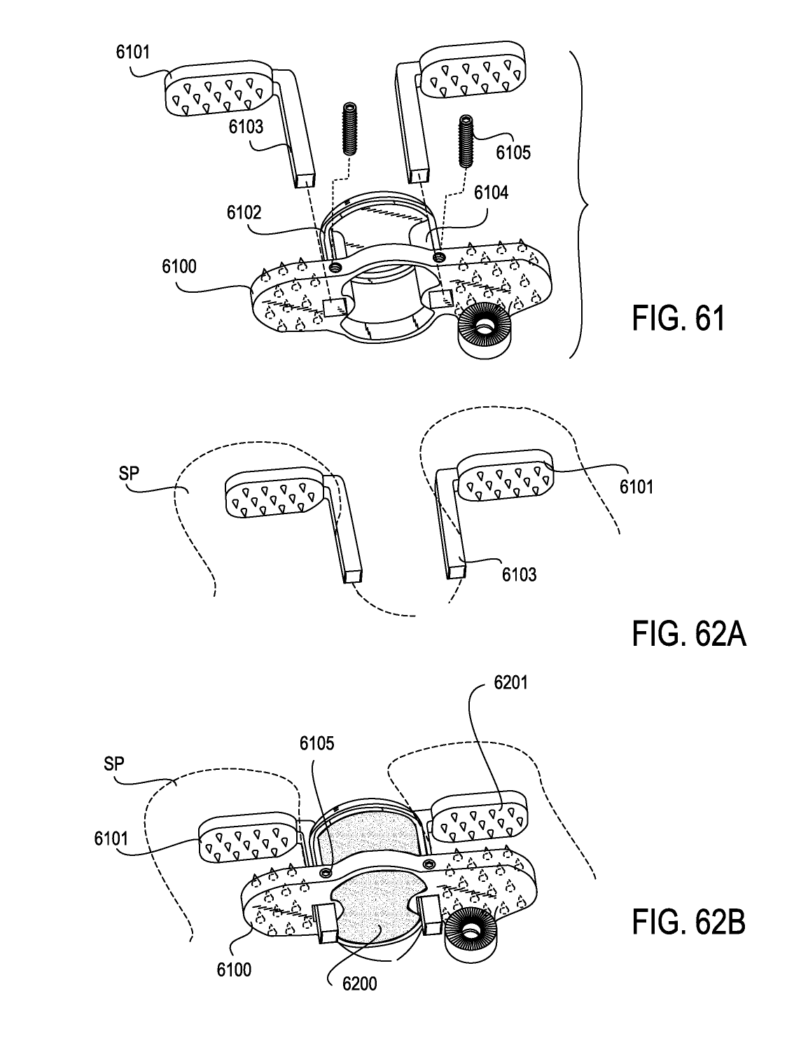

FIGS. 61 to 62B show an alternative exemplary embodiment of an interspinous fusion device. This device may be similar to the devices described above in that it is unilaterally deployable. As described above, the interspinous fusion device of this embodiment may include a fixation plate 6100 sized and configured to couple to a first lateral side of a spinous process and a coupling backing, coupled to the fixation plate and sized and configured to couple to the opposite lateral side of the spinous process. In this embodiment, the coupling backing comprises a paddle 6101. In some embodiments, as shown, the coupling backing comprises two paddles, one coupled to each of the two adjacent the spinous processes (SP) on opposite lateral side of the spinous processes. As shown, the paddle(s) may have a uniform thickness. Alternatively, the paddle(s) may be tapered along their length, width, and/or thickness. In some embodiments, as shown in FIG. 61, the fixation plate may further include a post 6102. The post may be coupled to the back side of the fixation plate (i.e. the side facing the spinous processes while implanted) and may be sized and configured to be positioned between two adjacent spinous processes. In some embodiments, the post may further function to distract the two spinous processes. As shown in FIGS. 62A and 62B, the post may also be sized and configured to receive a graft material 6200. The graft material may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the post may be filled with a filer material such as bone cement. As shown in FIG. 61, the post is configured such that is has a window or windows through which, the bone graft may contact the spinous processes and, in some instances, promote fusion. In some embodiments, the spinous processes may be decorticated prior to the implantation of the fixation plate and/or bone graft to also, in some instances, promote fusion. Furthermore, as illustrated in FIG. 61, the post may be configured to receive paddle extension(s) 6103 thereby coupling the paddle(s) to the fixation plate. For example, the post may include two grooves 6104 disposed along the length of the post. The grooves may be positioned on opposite sides of the post. Alternatively, the post and/or fixation plate may be coupled to the paddle extensions in any other suitable manner.

As shown, the paddle extensions of the current embodiment are sized such that they may pass between two adjacent spinous processes (FIG. 62A) and may extend from the fixation plate on the first lateral side of a spinous process to the paddle on the opposite lateral side of the spinous process (FIG. 62B). As shown in FIG. 62B, the paddle extensions may extend beyond the fixation plate in some configurations. In these instances, the end portion of the paddle extension may be bent, folded, removed, etc. Alternatively, the paddle extensions in this embodiment may couple to the post of the fixation plate without extending through the post to the fixation plate. Alternatively, the paddle may couple directly to the post and/or fixation plate without paddle extensions.

FIGS. 62A and 62B show an exemplary method for performing fusion unilaterally. In this example, the fixation device as described above may be sized and configured to be used in a method for performing an interlaminar fusion unilaterally. As shown in FIGS. 62A and 62B, an exemplary method for performing a unilateral spinal fusion may include the step of deploying a paddle having a paddle extension from a first lateral side of a spinous process. In some embodiments, a tool may be used to cut an opening through the interspinous ligament such that the paddle may be passed through the ligament from a first lateral side to the opposite lateral side of the spinous process. Alternatively, the paddle may be configured to be pushed through the interspinous directly. The interspinous ligament is a thin and membranous ligament that connects adjoining spinous processes and extends from the root to the apex of each process. In some embodiments, the opening may be created below the supraspinous ligament. The supraspinous ligament is a strong fibrous cord, which connects together the apices of the spinous processes from the seventh cervical vertebra to the sacrum. At the points of attachment of the supraspinous ligament to the tips of the spinous processes fibrocartilage is developed in the ligament. In some embodiments, an additional tool (or the paddle itself) may be used to dissect paraspinous muscle (muscle that runs next to, and roughly parallel with, the spine) from the lateral side of the spinous processes such that the paddle may couple to the bone of the spinous process.

As shown in FIG. 62A, in some embodiments, the inside of the paddle (i.e. the side facing the spinous processes while implanted) may include coupling members 6201. For example, as shown, the coupling members may be spikes or protrusions sized and configured to dig into or catch onto bone thereby aiding in the coupling of the paddle to the spinous process. Alternatively, the coupling members may be an abrasive surface, abrasive particles, or may include material such as bone cement or bone graft (including BMP for example).

As shown in FIG. 62B, an exemplary method for performing a unilateral spinal fusion may include the step of deploying a fixation plate having a post from a first lateral side of a spinous process. As described above, the post may be configured to receive and hold a graft material. The graft material may be implanted before, along with, or after the fixation plate. As shown in FIG. 62B, the post of the fixation plate is deployed between two adjacent spinous processes and over the paddle extensions, such that the paddle extensions are fed into the grooves of the post. The inside of the fixation plate (i.e. the side facing the spinous processes while implanted) may also include coupling members to aid in the coupling of the fixation plate to the spinous process. Once the fixation plate is coupled to the paddle and/or paddle extensions, the fixation plate and paddles may be approximated and/or coupled to the spinous processes. As described above, the coupling members of the fixation plate and/or paddle(s) may dig into or catch onto bone as the fixation plate and/or paddle(s) are approximated and/or moved toward the bone of the spinous processes. In some embodiments, a tool may inserted from the first lateral side of the spinous process and may be used to pull the paddle extensions from the first lateral side of the spinous process such that the paddle is pulled into the opposite side of the spinous process and/or a tool may be used to push the fixation plate into the first lateral side of the spinous process. A separate tool or the same tool may be used for each action or single tool may perform both actions simultaneously. As shown in FIGS. 61 and 62B a set screw may be inserted (from the first lateral side of the spinous process) into the fixation plate and/or paddle extensions to fix the position of the paddle extensions with respect to the fixation plate (or vice versa). Alternatively, the fixation plate and paddles may be connected in any other suitable fashion.

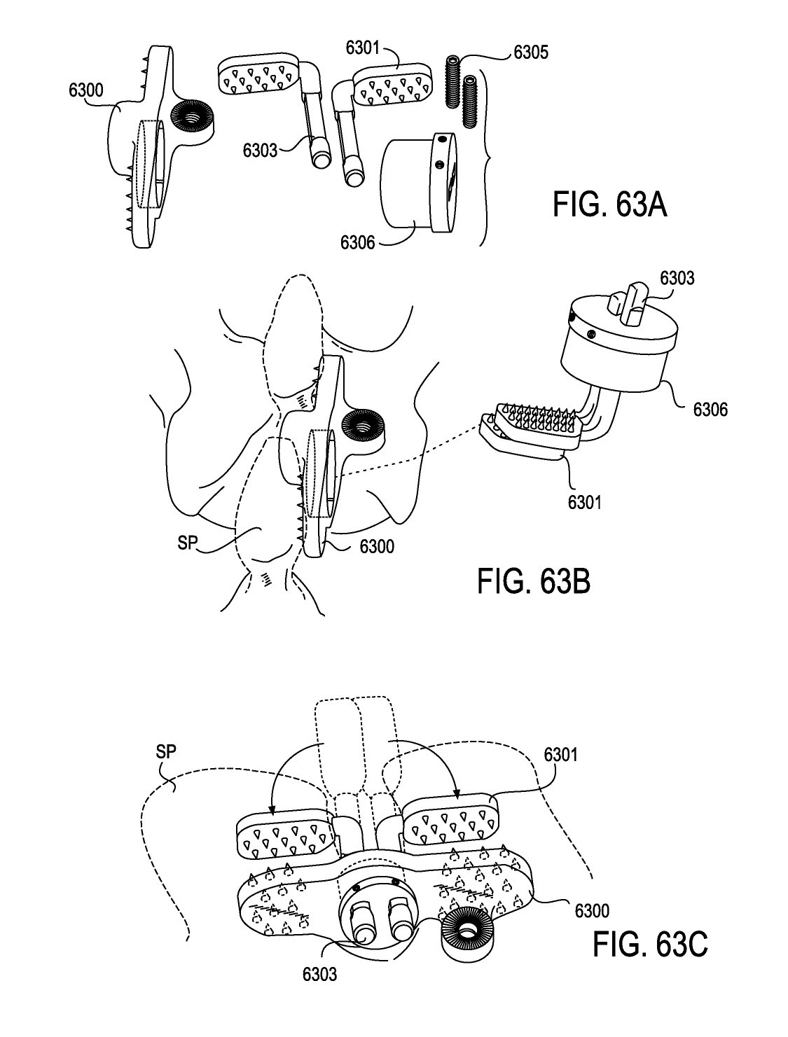

FIGS. 63A to 63C show an alternative exemplary embodiment of an interspinous fusion device and method for performing a unilateral spinal fusion. This device and method may be similar to the devices described above in reference to FIGS. 61 to 62B with the addition of a body element 6306 that may be coupled to the paddle extensions prior to implantation, such that the paddles may be deployed with the body through the fixation plate. As shown in FIG. 63A an exemplary method for performing a unilateral spinal fusion may include the step of deploying a fixation plate from a first lateral side of a spinous process. As shown, the fixation plate is configured to couple to a first lateral side of two adjacent spinous processes. The fixation plate defines an opening between the two spinous processes such that a body and/or a graft may be implanted through the fixation plate and between the spinous processes. As shown in FIG. 63B, the fusion device may include a body. The body is sized and configured to be implanted through the opening of the fixation plate and between the spinous processes. The body may include a lip or edge toward one end, such that the body cannot be pushed all the way through the fixation plate. In some embodiments, the body may be made out of a bone graft material. The bone graft material may be machined or otherwise shaped. Alternatively, the bone graft material may be a mass of bone, such as chips or bone pieces, flexible cancellous, materials containing DBM, and/or materials containing bone morphogenetic proteins (BMP). The bone graft may contact the adjacent spinous processes and, in some instances, promote fusion. Alternatively, the body may be metallic or plastic. For example, the body may be made out of PEEK. As shown, the body defines a channel or channels that are sized and configured to receive the paddle extensions as described above with reference to FIGS. 61 to 62B. As shown in FIG. 63B, the paddles and paddle extensions may be positioned within the body such that the paddles are in a first orientation. In the first orientation the paddles are in a vertical position such that they may be passed between the spinous processes (for example, through an opening in the interspinous ligament as described above) from the first lateral side of the spinous processes. Once the paddles (and body) are in position between the adjacent spinous processes, the paddle extensions may be rotated within the body such that the paddles move to a second orientation, as shown in 63C. In the second orientation, the paddles are in a horizontal configuration such that they may each couple to an opposite lateral side of a spinous process. The paddle extensions may be rotated (using a tool for example) from the first lateral side of the spinous processes. Once the paddles are in the second orientation, the paddle extensions may be pulled from the first lateral side of the spinous process such that the paddle is pulled into the opposite side of the spinous process, as shown in FIG. 63C. Alternatively, a tool may inserted from the first lateral side of the spinous process and may be used to push the fixation plate into the first lateral side of the spinous process. A separate tool or the same tool may be used for each action or single tool may perform both actions simultaneously. A set screw may be inserted (from the first lateral side of the spinous process) into the body and/or the fixation plate and/or paddle extensions to fix the position of the paddle extensions with respect to the fixation plate (or vice versa). Alternatively, the fixation plate and paddles may be connected in any other suitable fashion.

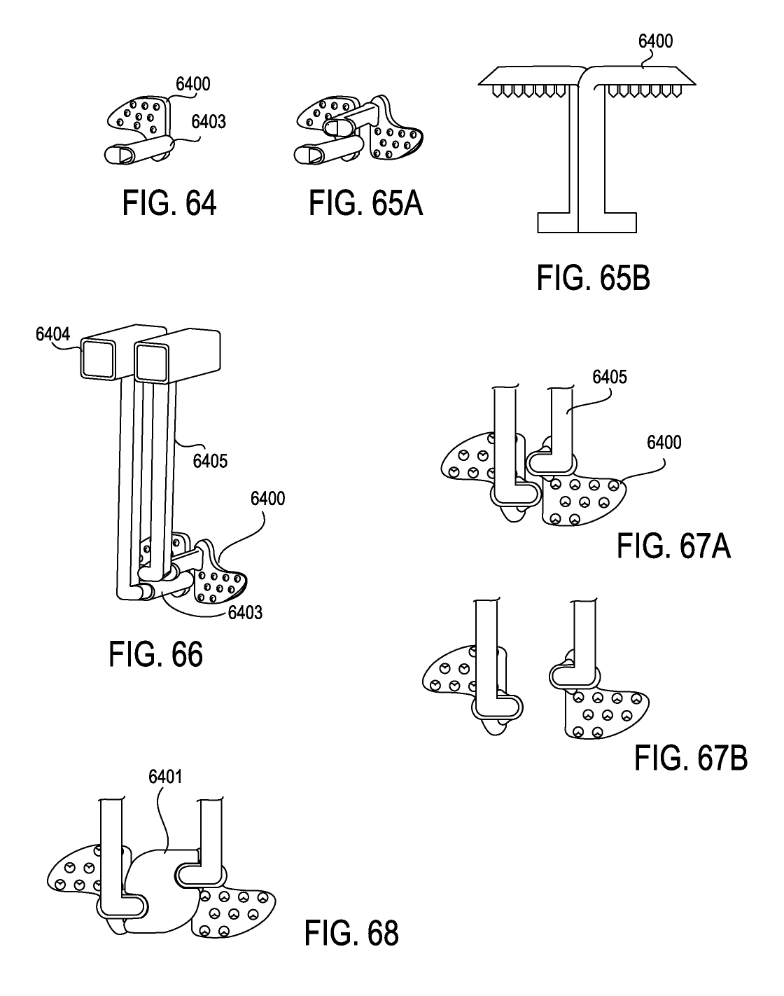

FIGS. 64 to 72 show an exemplary embodiment of an interspinous fusion device, specifically one that includes at least one paddle 6400 and that may be deployable unilaterally. This device and method may be similar to the devices described above in reference to FIGS. 61 to 62B, however the fixation plate may not have an integrated post—alternatively, the device may include a separate spacer 6401 (optional) and fixation plate 6402. As described above, the interspinous fusion device of this embodiment may include a fixation plate sized and configured to couple to a first lateral side of a spinous process and at least one paddle, coupled to the fixation plate and sized and configured to couple to the opposite lateral side of the spinous process. In some embodiments, as shown, the device comprises two paddles, one configured to couple to each of the two adjacent the spinous processes on opposite lateral side of the spinous processes. As shown, the paddle(s) may have a uniform thickness. Alternatively, as shown in FIG. 65B, the paddle(s) may be tapered along their length, width, and/or thickness. This taper may facilitate tissue dissection. Alternatively, an additional tool may be used to dissect paraspinous muscle (muscle that runs next to, and roughly parallel with, the spine) from the lateral side of the spinous processes such that the paddles (and/or fixation plate) may couple to the bone of the spinous processes. As shown in FIGS. 64 and 65A, the paddle may have a geometry configured such that multiple devices may be implanted in adjacent levels. For example, the left paddle in FIG. 65A, has a recess toward the top of the paddle. This recess is sized and configured to receive a right paddle implanted at the adjacent level, which has a recess toward the bottom of the paddle that is sized and configured to receive the left paddle. As shown in FIG. 65A, the device may include two paddles (a left and a right). In some embodiments, the left and the right paddle may have different sizes or configurations. Alternatively, as shown, the left and right paddle may be the same but rotated 180 degrees from one another while implanted. As shown in FIGS. 65A and 65B, once implanted, the paddle extensions 6403 of the left and right paddle may overlap one another along the length of the extensions. This may be beneficial in spines where the two adjacent spinous processes are close to one another and there is not room for two paddle extensions to be implanted between the spinous processes side by side. As shown in FIGS. 64 to 65B, the paddle extensions of the current exemplary embodiment are sized such that they may pass between two adjacent spinous processes and may extend from the fixation plate on the first lateral side of a spinous process to the paddle on the opposite lateral side of the spinous process.

As shown in FIGS. 66 to 67B, the paddles and/or paddle extensions may be coupled to a tool 6404 that may facilitate the distraction (separation) of the paddles from one another. In some embodiments, the paddles will be separated from one another such that they separate or distract two adjacent spinous processes from one another. This separation of the spinous processes may reduce intradiscal pressure, stretch apart a collapsed disc space thereby enlarging the spinal canal, decompress the spinal cord or spinal nerves, relieve back pain, treat symptomatic spinal stenosis (such as lumbar spinal stenosis), and/or perform any combination thereof. As shown in FIG. 66, the distraction arms 6405 may be coupled to the paddle extensions. The arms may be coupled to the paddles before, during, or after implantation. As shown in FIG. 66, the distraction arms may include a coupling member such that the arms may be coupled to a conventional instrument such as a McCulloch Retractor, for example. As the arms are moved apart from one another, the paddle(s) and paddle extension(s) will be moved from one another such that the adjacent spinous processes are separated. As described below, the device may further include a spacer. As shown in FIG. 68, the spacer may function to maintain the distraction of the spinous processes; alternatively, the distraction may be released once the spacer and/or the fixation plate are implanted. Alternatively, rather than by using distraction arms, the spacer may be sized and configured to separate the paddles and/or paddle extensions as the spacer is pushed into place over the paddle extensions. For example, the spacer may be tapered along its length (bullet shaped, for example).

In some embodiments, as shown in FIG. 68, the device may further include a spacer. The spacer may be sized and configured to be positioned between two adjacent spinous processes. The spacer may be made out of a graft material or alternatively may be sized and configured to receive a graft material. The graft material may be made out of a bone graft material. The bone graft material may be machined or otherwise shaped. Alternatively, the bone graft material may be a mass of bone, such as chips or bone pieces, flexible cancellous, materials containing DBM, and/or materials containing bone morphogenetic proteins (BMP). Alternatively, the spacer may be filled with a filer material such as bone cement. The bone graft may contact the spinous processes and, in some instances, promote fusion. In some embodiments, the spinous processes may be decorticated prior to the implantation of the fixation plate and/or bone graft to also, in some instances, promote fusion. Furthermore, as illustrated in FIG. 68, the spacer 6401 may be configured to slide over the paddle extension(s) thereby holding the paddle(s) in a distracted configuration as described above. For example, the spacer may include two grooves disposed along the length of the spacer. The grooves may be positioned on opposite sides of the spacer. Alternatively, the spacer and/or fixation plate may be coupled to the paddle extensions in any other suitable manner. The spacer may function to distract the paddles and/or to maintain a distraction of the paddles (and/or spinous process). Therefore, a spacer may be selected from a plurality of spacers having the desired size for the distraction desired. For example, the spinous processes may be distracted anywhere from 0 mm to 16 mm. For example, they may be distracted 5 mm, 6 mm, 8 mm, 10 mm, 12 mm, 14 mm, 16 mm, or to any other suitable distance.

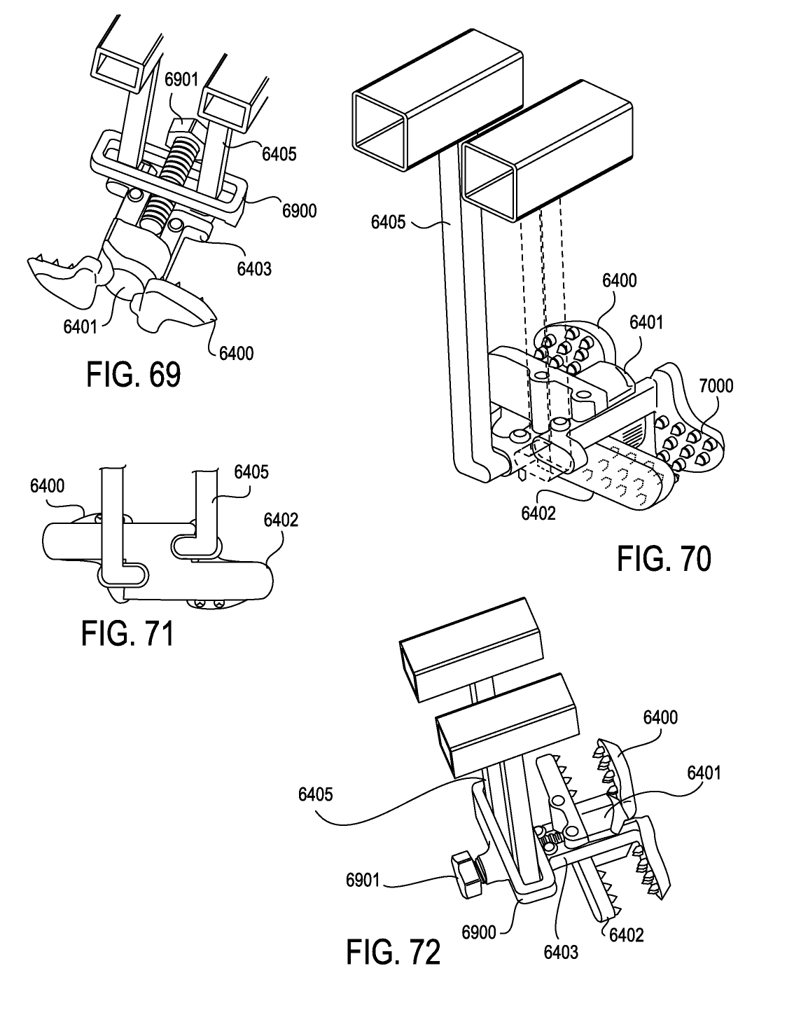

As shown in FIG. 69, a tool may be used to push the spacer onto the paddle extensions. For example, as shown, a guide 6900 may be coupled to the distraction arms. The slot of the guide may be sized to allow the arms to be distracted to any suitable width. In some embodiments, the arms may be distracted anywhere from 0 mm to 16 mm. For example, the arms may be distracted 5 mm, 6 mm, 8 mm, 10 mm, 12 mm, 14 mm, 16 mm, or to any other suitable distance. Alternatively, the guide may have an open end such that the arms may be distracted out of the slot of the guide. The pusher 6901 may be coupled to the guide and may function to move the spacer with respect to the guide. For example, the pusher may be a threaded rod or screw. As the pusher is rotated, it will move against the spacer and push the spacer toward the paddles and the opposite lateral side of the spinous processes.

As shown in FIGS. 70 to 72, an exemplary method for performing a unilateral spinal fusion may include the step of deploying a fixation plate from a first lateral side of a spinous process and coupling it to the paddles and/or extensions. As shown in FIG. 71, the fixation plate is deployed between the two distraction arms and over the paddle extensions, such that the paddle extensions are fed into the grooves of the fixation plate. The fixation plate may be fed between the two arms at an angle, and then rotated such that the inside face of the fixation plate is adjacent to the lateral side of the spinous processes. This may also be in an orientation that is substantially parallel to the inside face of the paddles. Alternatively, the distraction arms may be removed (temporarily or for the remainder of the procedure) prior to the implantation of the fixation plate.

As shown in FIG. 70, the inside of the fixation plate (i.e. the side facing the spinous processes while implanted) may also include coupling members 7000 to aid in the coupling of the fixation plate to the spinous process. Once the fixation plate is coupled to the paddle and/or paddle extensions, the fixation plate and paddles may be approximated and/or coupled to the spinous processes, as shown in FIG. 70. As described above, the coupling members of the fixation plate and/or paddle(s) may dig into or catch onto bone as the fixation plate and/or paddle(s) are approximated and/or moved toward the bone of the spinous processes. In some embodiments, as shown in FIG. 72, a tool such as a pusher and guide as described above may inserted from the first lateral side of the spinous process and may be used to pull the paddle extensions from the first lateral side of the spinous process such that the paddle is pulled into the opposite side of the spinous process and/or push the fixation plate into the first lateral side of the spinous process. As shown in FIG. 72, the paddle extensions may be pinned to the distraction arms and/or the guide. The paddles may be held substantially fixed against the opposite lateral side of the spinous processes by the distraction arms, while the fixation plate is pushed against the first lateral side of the spinous processes by the pusher. A separate tool or the same tool may be used for each action or single tool may perform both actions simultaneously. Alternatively, a tool such as a pliers or scissor may be placed between the guide and the fixation plate and may be opened such that a first arm of the tool pushes the fixation plate away from the guide and toward the spinous processes. The second arm of the tool may push the guide away from the fixation plate, thereby pulling the paddles into the opposite side of the spinous processes.

As shown in FIG. 72 a locking mechanism, such as a set screw, may be inserted (from the first lateral side of the spinous process) into the fixation plate and/or paddle extensions to fix the position of the paddle extensions with respect to the fixation plate (or vice versa). Alternatively, the fixation plate and paddles may be connected in any other suitable fashion.

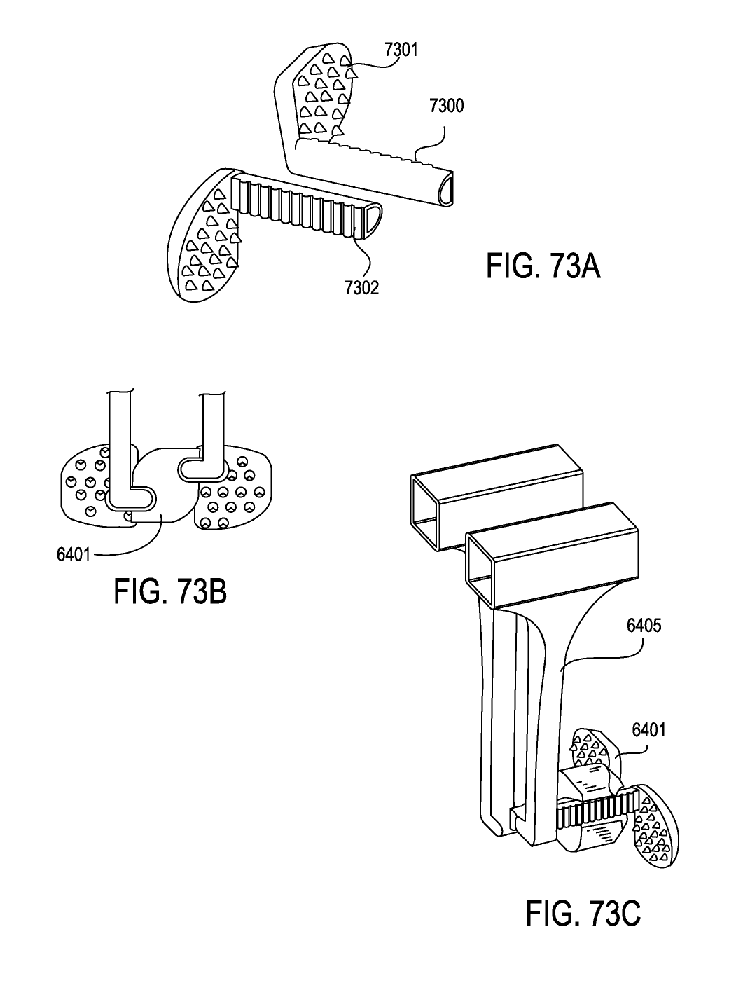

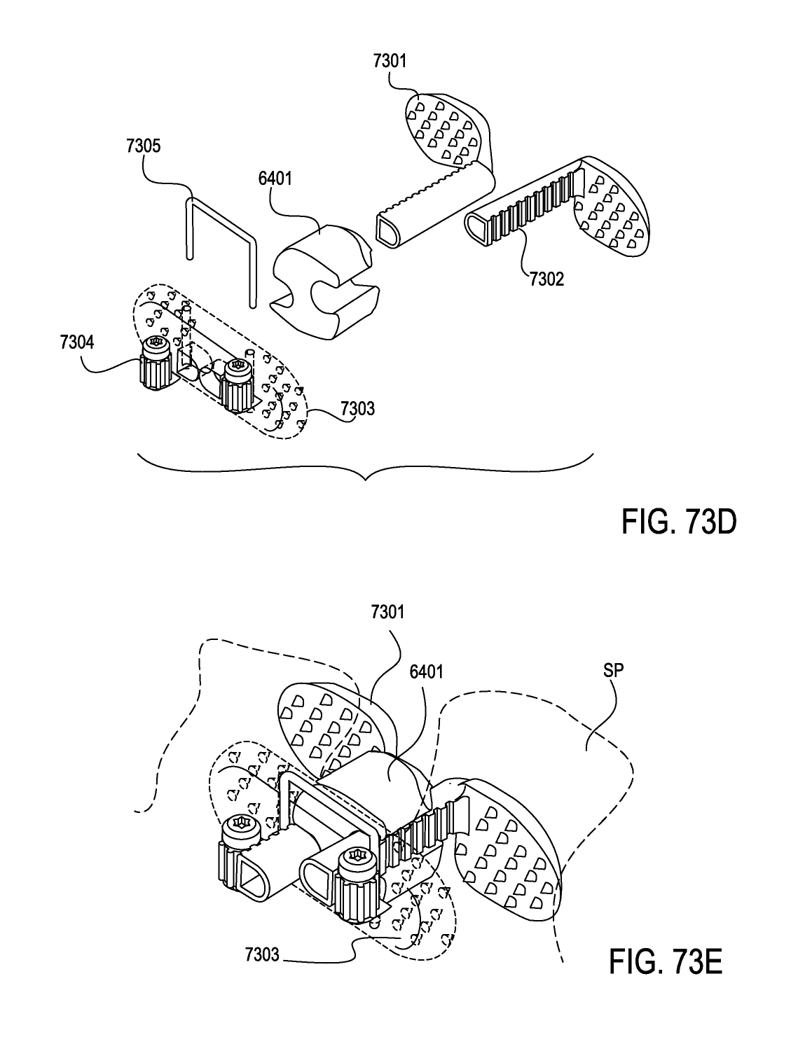

FIGS. 73A to 73E show an exemplary embodiment of an interspinous fusion device, specifically one that includes at least one paddle and that may be deployable unilaterally. This device and method may be similar to the devices described above in reference to FIGS. 64 to 72, with an alternative embodiment of the mechanism that may be used to pull the paddle extensions 7300 from the first lateral side of the spinous process such that the paddle 7301 is pulled into the opposite side of the spinous process and/or push the fixation plate 7303 into the first lateral side of the spinous process. As shown in FIGS. 73A and 73C, the paddle extensions include teeth 7302 distributed along the length of the extension. In this embodiment, the paddle extensions may function as a rack, of a rack and pinion gear combination. As shown in FIGS. 73C and 73D, the fixation plate may comprise the pinion gears 7304 of the clamping mechanism of the device. As the pinion gears are rotated, the paddle extensions will pull the paddles into the opposite lateral side of the spinous processes and/or the fixation plate will move toward the first lateral side of the spinous process. Once the fixation plate and the paddles are clamped against the spinous processes, a locking mechanism, such as a U-shaped pin 7305, as shown in FIG. 73D, may be implanted such that the fixation plate is pinned to the paddle extensions.

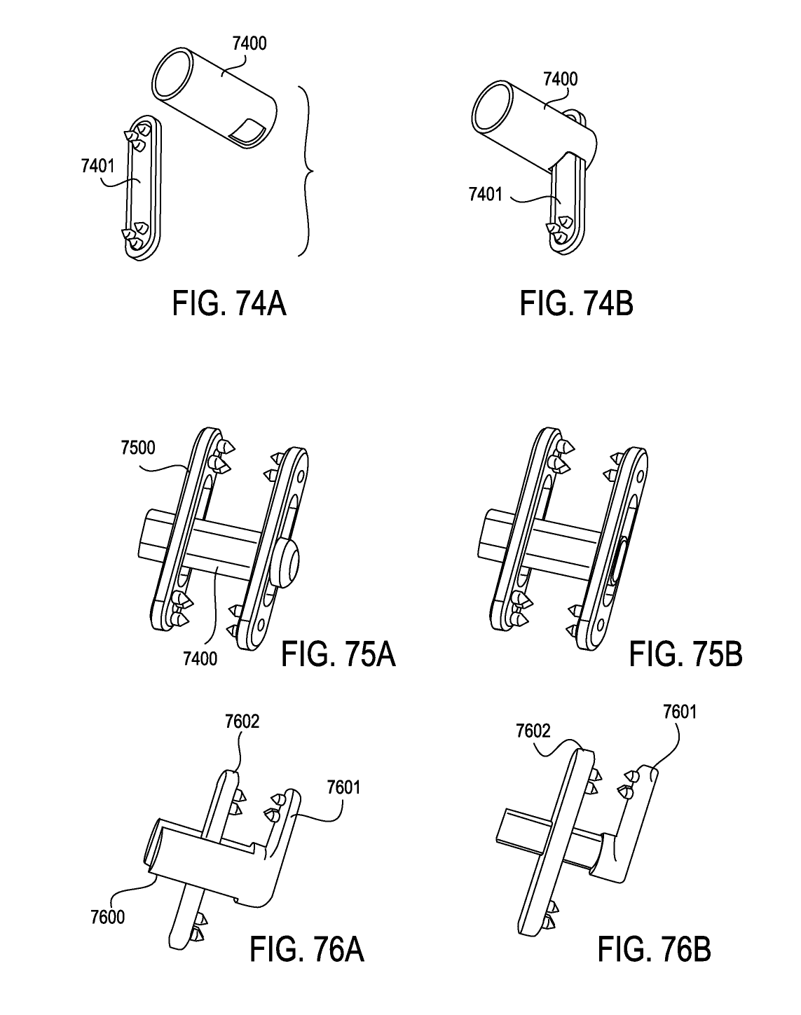

FIGS. 74A to 76B show exemplary embodiments of interspinous fusion devices, specifically one that may be deployable unilaterally. In this example, the device includes a slideable fixation plate. As shown in FIG. 74A, the device includes a post 7400, and a fixation plate 7401 that is slideable with respect to the post. For example, as shown in FIG. 74B, the device is in a first configuration. In this configuration, the fixation plate is positioned with respect to the post, such that the post and plate form an L-shape. The L-shaped device may be deployed from a first lateral side of the spinous processes such that the fixation plate may be fed between two adjacent spinous processes to the opposite lateral side of the spinous processes. Once on the opposite lateral side of the spinous processes (with the post between the spinous processes), the fixation plate may be moved to a second configuration, as shown in FIGS. 75A or 75B. In this configuration, the center of the fixation plate is moved toward the post such that the post and plate form a T-shape. The fixation plate in the T-shaped configuration may couple to the opposite lateral side of two adjacent spinous processes. A second fixation plate 7500 may be coupled to the post (from the first lateral side of the spinous processes) such that it couples to the first lateral side of the spinous process. The second fixation plate may be coupled to the device such that the device is in an H-shaped configuration.

In an alternative embodiment, as shown in FIGS. 76A and 76B, the device includes a post 7600, and a half fixation plate 7601 that is coupled to the post. For example, in a first configuration, one end of the half fixation plate is coupled to an end of the post, such that the post and plate form an L-shape. The L-shaped device may be deployed from a first lateral side of the spinous processes such that the fixation plate may be fed between two adjacent spinous processes to the opposite lateral side of the spinous processes. A second fixation plate 7602 may be coupled to the post (from the first lateral side of the spinous processes) such that it couples to the first lateral side of the spinous process. The second fixation plate may be coupled to the device such that the device is in a lower-case-h-shaped configuration.

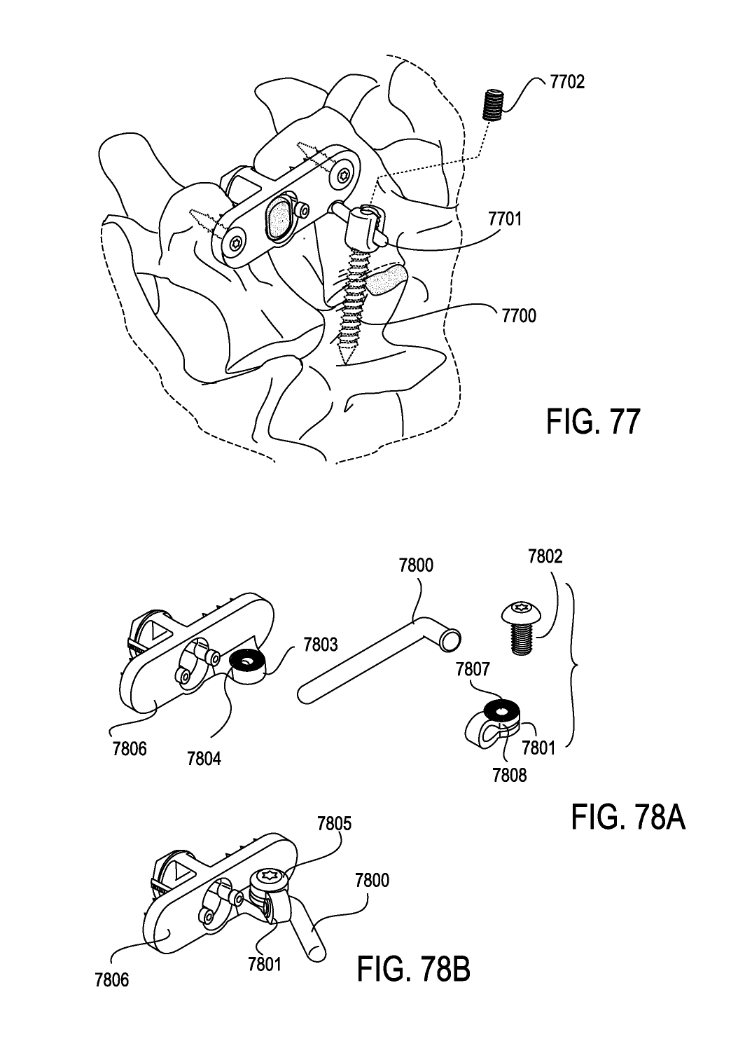

FIG. 77 shows an exemplary embodiment of an interspinous fusion device, specifically one that may be deployable unilaterally. In this example, the fixation device may include, for example, an ipsilateral trans-facet interpedicular screw 7700, a pedicle screw, or a trans-facet screw. The fixation device and system described here, in reference to FIG. 77, may be similar to the device described in reference to FIGS. 50A to 51 with alternative variations of the bracket and the fixation screw. In this variation, the bracket comprises a post or rod 7701 that is coupled to the front side of the fixation plate. The rod extends from the front side of the fixation plate toward the facet joint and/or pedicle. The distal free end of the rod is sized and configured to be placed onto or into the fixation screw head. As shown in FIG. 77, the fixation screw head of this embodiment includes a tulip-head or otherwise slotted screw head. The slot of the screw head is sized and configured to receive the rod of the fixation plate. The slot of the screw head may also be threaded or otherwise configured to also receive a locking screw 7702. Once the rod is positioned within the screw head (or coupled to the screw head in any other suitable configuration) a locking screw may be coupled to the screw head such that it locks the rod into position with respect to the screw head.

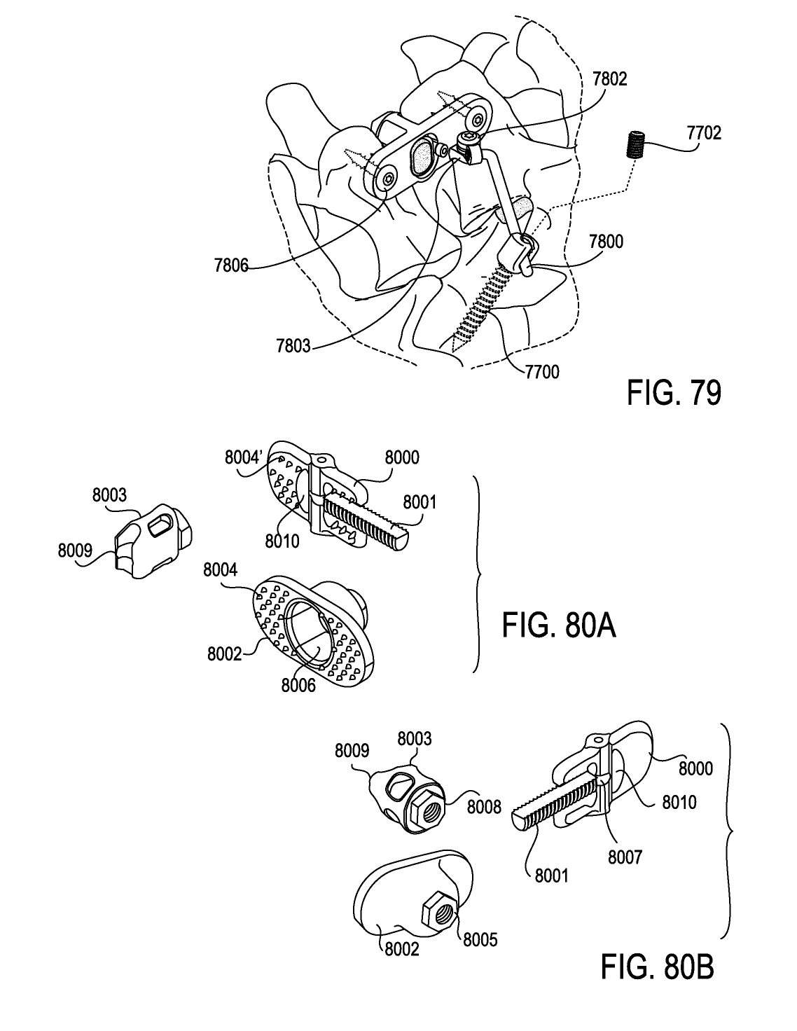

FIGS. 78A to 79 show an exemplary embodiment of an interspinous fusion device, specifically one that may be deployable unilaterally. The fixation device and system described here in reference to FIGS. 78A to 79 may be similar to the device described in reference to FIG. 77 with alternative variations of the bracket (e.g. rod) and the mechanism through which it is coupled to the fixation plate. As shown in FIG. 78A, in this variation, like the variation described above, the bracket comprises a post or rod 7800 that is coupled to the front side of the fixation plate. The rod extends from the front side of the fixation plate toward the facet joint and/or pedicle. The distal free end of the rod is sized and configured to be placed onto or into the fixation screw head. In this embodiment however, the rod is coupled to the fixation plate by way of an adjustable joint. The joint comprises an extension 7803, a fastener 7801, and a screw 7802. As shown in FIGS. 78A and 78B, the extension extends from the front side of the fixation plate 7806. In some embodiments, as shown, the extension may define a hole 7804 sized and configured to receive a screw 7805. Additionally, in some embodiments, the extension may be configured to couple to a fastener. For example, in some embodiments, the extension and the fastener may have mating surfaces that include a series of grooves or ridges. The grooves and/or ridges may prevent rotation of the extension and/or fastener with respect to one another when they are tightened against one another, by the screw for example. By rotating the fastener with respect to the extension (and fixation plate) about the screw (or axis of the screw), the yaw of the rod may be adjusted. The system may include multiple rods, or a single rod of any suitable length. This may allow for variability between patients, specifically the variability in the distance from the spinous process to the facet joint and/or pedicle.

As shown in FIG. 78B, the fastener may be a clevis joint, locking ring, or any other suitable joint. The fastener may define a first hole 7807 sized and configured to receive the screw, and a second hole 7808 sized and configured to receive the rod. The axis of the screw hole may be substantially perpendicular to the axis of the rod hole. Alternatively, the axes may be at any other suitable angle to one another. The rod hole may also include a series of grooves or ridges. The grooves and/or ridges may prevent rotation of the rod and/or fastener with respect to one another when they are tightened against one another, by the screw for example. By rotating the rod with respect to the fastener (about the axis of the rod hole), the pitch of the rod may be adjusted.

As shown in FIG. 79, the interspinous fusion device of this embodiment may be implanted similarly to other embodiments described herein. As shown in FIG. 79, once the fixation screw and fixation plate are implanted, a rod may be coupled to the fixation plate via the adjustable joint and the rod may be coupled to the fixation screw head via a locking screw. The fastener (and rod) may be coupled to the extension and rotated with respect to the extension to adjust the yaw of the rod such that the rod may be coupled to the fixation screw head. Furthermore, the rod may be rotated with respect to the fastener to adjust the pitch of the rod such that the rod may be coupled to the fixation screw head. This adjustability may allow for variability between patients, specifically the variability in the position of the facet joint and/or pedicle with respect to the spinous process. In some embodiments, the rod may be selected for a specific patient from a plurality of rods having varying lengths. Once the adjustable joint and rod are positioned correctly, the screw may be tightened such that the elements of the joint are fixed into place and the rod may not be allowed to move with respect to the fixation plate and/or the fixation screw head.

FIGS. 80A and 80B show an exemplary embodiment of an interspinous fusion device, specifically one that may be deployable unilaterally. As shown, the devices and systems may include a first fixation plate 8000 having a rod, a second fixation plate 8002, and a barrel 8003. The second fixation plate may be sized and configured to couple to a fist lateral side of a spinous process. The second fixation plate may include coupling members 8004 positioned on the back side of the fixation plate (i.e. the side facing the spinous processes while implanted) and a locking mechanism 8005 on the front side of the plate. As described above, the coupling members may be spikes or protrusions sized and configured to dig into or catch onto bone thereby aiding in the coupling of the fixation plate to the spinous process(es). The second fixation plate may include a hollow region 8006 sized and configured to receive a rod and/or barrel as described below.

The first fixation plate may be coupled to a rod via a joint 8007 such that the plate is pivotable with respect to the rod about the joint. As shown, the first fixation plate may also include coupling members 8004′ positioned on the back side of the first fixation plate. As shown, the rod may be threaded along its length, or a portion thereof. The rod may have a non circular cross section, as shown, such that the barrel does not rotate about the rod as described below. The barrel, as shown, includes a rod locking mechanism on a first side (locking nut 8008, as shown in FIG. 80B) and a plate locking mechanism on a second side (tabs 8009, as shown in FIG. 80A). As shown, the plate locking mechanism comprises two tabs that extend from the end of the barrel. These tabs are sized and configured to couple to the slots 8010 of the first fixation plate and lock the plate in position with respect to the rod and/or the spinous processes. The locking nut coupled to the barrel functions to screw onto the rod and lock the barrel in position with respect to the first fixation plate. Additionally, the barrel may be may be sized and configured to be positioned between two adjacent spinous processes. In some embodiments, the barrel may further function to distract the two spinous processes. The barrel may include a hollow region sized and configured to receive the rod. In some embodiments, the hollow region of the barrel has a cross sectional geometry to match that of the rod such that the barrel will not rotate about the rod. Furthermore, as shown in FIGS. 80A and 80B, the barrel may also be sized and configured to receive a graft material. The graft material may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the barrel may be filled with a filer material such as bone cement. As shown in FIGS. 80A and 80B, the barrel is configured such that is has a window or windows through which, the bone graft may contact the spinous processes and, in some instances, promote fusion. In some embodiments, the spinous processes may be decorticated prior to the implantation of the fixation plate and/or bone graft to also, in some instances, promote fusion. In some embodiments, the graft material may be placed between the first and second fixation plates without the use of the barrel.

The barrel is an optional element that may be coupled to the first fixation plate, rod, and/or second fixation plate. Alternatively, the second fixation plate may be coupled to the first fixation plate and/or rod without a barrel. The barrel may function similarly to the post 5503, as described in reference to FIGS. 55A to 55C. As described, the barrel may be sized and configured to be positioned between two adjacent spinous processes. For example, as shown, the barrel may be substantially cylindrically shaped and may be disposed over a portion of the length of the rod. Alternatively, the barrel may have any suitable shape such as elliptical or rectangular. In some embodiments, the barrel may be positioned toward the anterior portion of the spinous process and/or between two adjacent laminas. This may be advantageous due to the fact that there may be more substantial bone surfaces for the barrel to contact in this area, furthermore, these portions of the spinous processes/lamina may be stronger and more stable to receive forces from the barrel. In some embodiments, the barrel may receive the majority of the loads from the spinous processes.

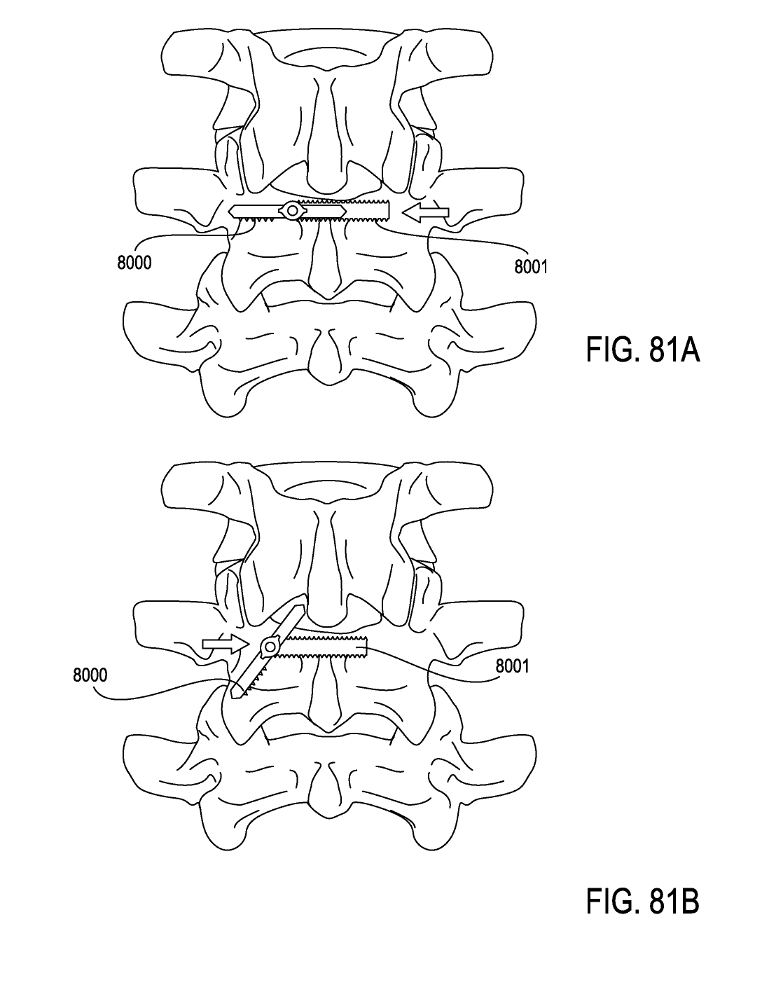

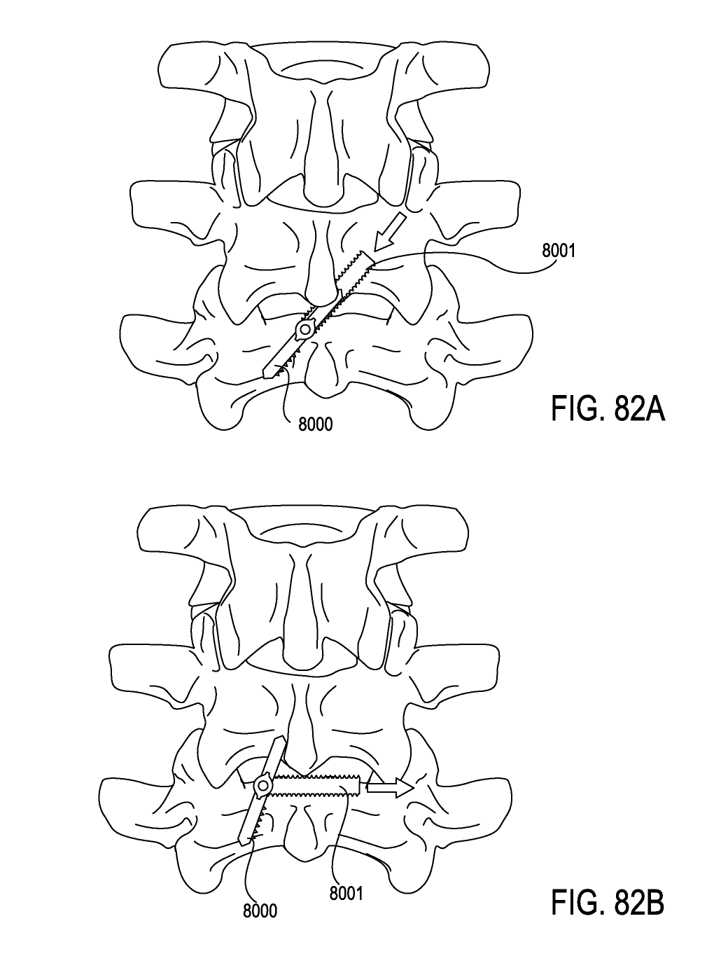

As shown in FIGS. 81A and 81B, an exemplary method for performing a unilateral spinal fusion may include the step of deploying a first fixation plate from a first lateral side of a spinous process to the opposite lateral side of the spinous process. As shown, the first fixation plate is rotated with respect to the rod such that they are aligned along the same axis and can fit between two adjacent spinous processes. Alternatively, the plate and the rod may be at an angle to one another, as long as the plate can fit through between the spinous processes. As shown in FIG. 81A, the plate is deployed between the spinous processes at an angle that is about perpendicular the length (cephalad to caudal) of the spine. Alternatively, as shown in FIGS. 82A and 82B for example, the fixation plate may be inserted between the spinous processes from cephalad aspect of spine, at an angle to the length of the spine. For example, the plate may be inserted at a 60 degree, 45 degree, 30 degree, 15 degree, or smaller angle with respect to the length of the spine.

In some embodiments, a tool may be used to cut an opening through the interspinous ligament such that the plate (and rod) may be passed through the ligament from a first lateral side to the opposite lateral side of the spinous process. Alternatively, the plate may be configured to be pushed through the interspinous directly. The interspinous ligament is a thin and membranous ligament that connects adjoining spinous processes and extends from the root to the apex of each process. In some embodiments, the opening may be created below the supraspinous ligament. The supraspinous ligament is a strong fibrous cord, which connects together the apices of the spinous processes from the seventh cervical vertebra to the sacrum. At the points of attachment of the supraspinous ligament to the tips of the spinous processes fibrocartilage is developed in the ligament. In some embodiments, an additional tool (or the plate itself) may be used to dissect paraspinous muscle (muscle that runs next to, and roughly parallel with, the spine) from the lateral side of the spinous processes such that the plate may couple to the bone of the spinous process. For example, as shown, the plate may include a sharp and/or tapered leading edge such that as the plate is implanted, it may dissect the paraspinous muscle from the side of the spinous processes. The plate may dissect muscle away for example, as the plate is moved against the side of the spinous processes, as shown in FIGS. 81B and 82A and 82B. As shown, once the plate is implanted past the spinous processes, the rod may be pulled back such that the plate is pulled back against the opposite lateral side of the spinous processes. As the rod is pulled back, the plate is rotated, against the spinous process, with respect to the rod.

In some embodiments, the device may be deployed bilaterally. For example, the method for performing a bilateral spinal fusion may include the step of deploying the second fixation plate from the opposite lateral side of the spinous process to position the plate against the opposite lateral side of the spinous process. In this embodiment, the plate may be coupled to the rod, and the rod may be positioned such that it is perpendicular to the fixation plate. Alternatively, the rod may not be coupled to the plate while the plate is implanted or the rod may be positioned with respect to the plate at any suitable angle.

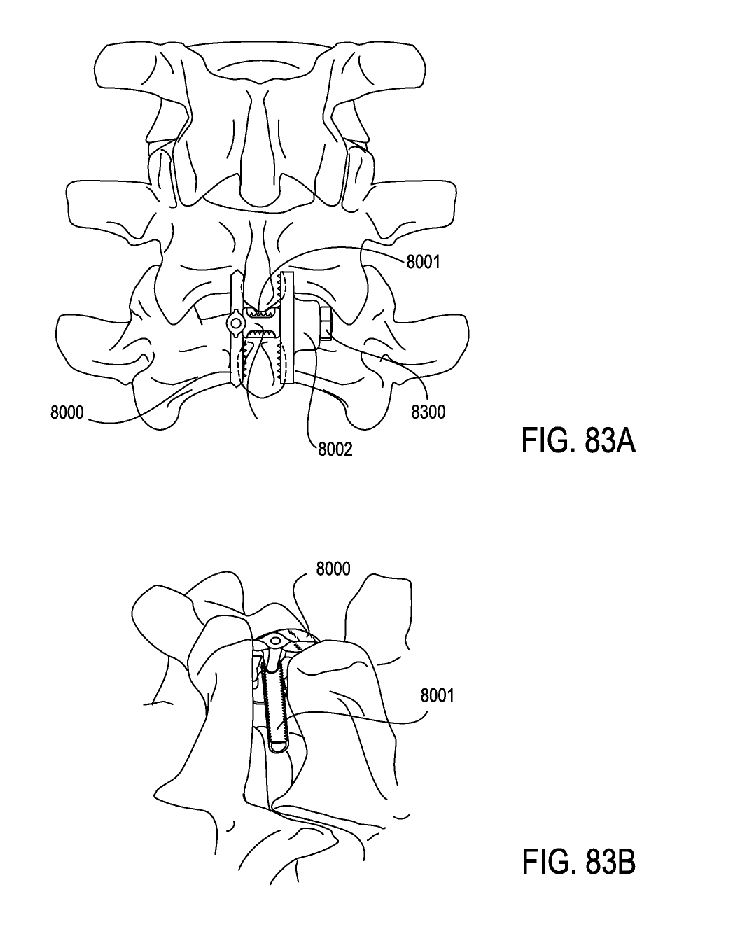

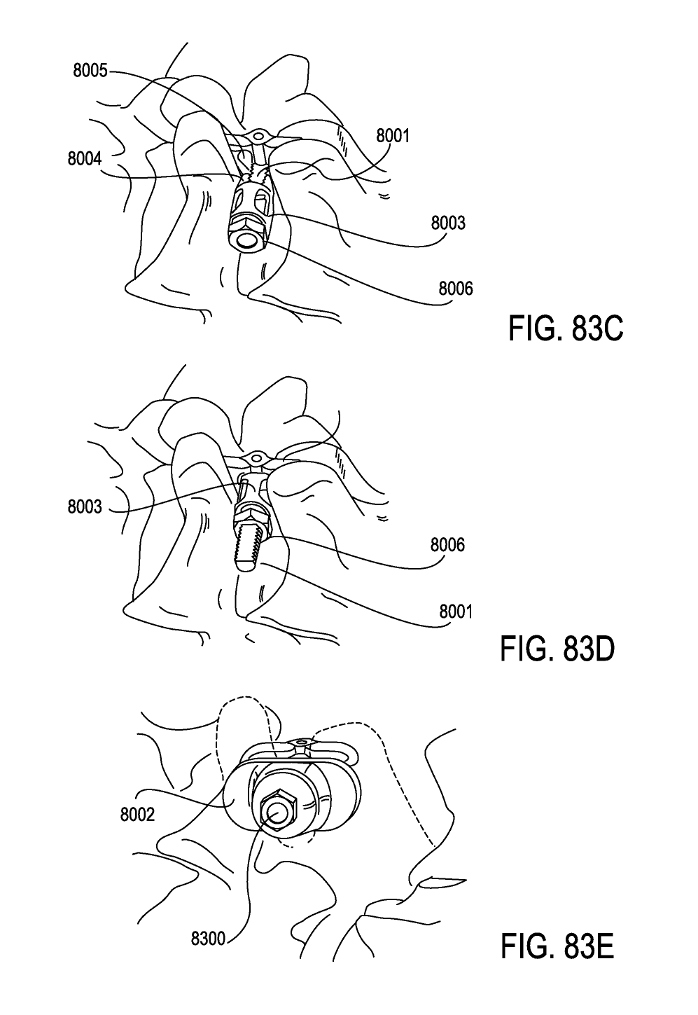

Once in position, as shown in FIG. 83B, the first fixation plate 8000 is about perpendicular to the rod 8001. In some embodiments, depending on the anatomy of the patient and the size and shape of the spinous processes, the fixation plate may not be exactly perpendicular to the rod. As shown in FIGS. 83B and 83D, the barrel 8003 may then be fed onto the rod from the first (same) lateral side of the spinous processes. As described above, the barrel may also function to distract the two adjacent spinous processes. As the barrel is slid onto the rod, the tabs 8004 will couple to the slots 8005 of the fixation plate, and fix the plate with respect to the rod. Once the barrel is flush with the fixation plate and/or the tabs are coupled to the plate, the locking nut 8006, or other suitable locking mechanism, will be tightened down to hold the barrel in position with respect to the rod and/or the fixation plate. As shown in FIGS. 83A and 83E, the second fixation plate 8002 is then fed over the rod and/or the barrel from the first (same) lateral side of the spinous processes. Once the second fixation plate is positioned against the first lateral side of the spinous processes, the plate may then be locked into place. As shown in FIGS. 83A and 83E, a locking nut 8300 may be used, alternatively, any other suitable locking mechanism may be used to lock the plate into plate with respect to the spinous processes and/or the first fixation plate. In some embodiments, as the locking mechanism is tightened onto the rod, the mechanism will pull the rod, and therefore the first fixation plate, toward the first lateral side of the spinous processes, such that the back side (and the coupling members) of the first fixation plate are pulled into the opposite side of the spinous processes. By pulling the first fixation plate into the opposite side and pushing the first fixation plate (and the coupling members) into the first side of the spinous processes, the device is compressed onto the spinous processes.

In some embodiments, the device may be deployed bilaterally. For example, the method for performing a bilateral spinal fusion may include the step of deploying the second fixation plate from the opposite lateral side of the spinous process to position the plate against the opposite lateral side of the spinous process. In this embodiment, the plate may be coupled to the rod, and the rod may be positioned such that it is perpendicular to the fixation plate. Alternatively, the rod may not be coupled to the plate while the plate is implanted or the rod may be positioned with respect to the plate at any suitable angle.

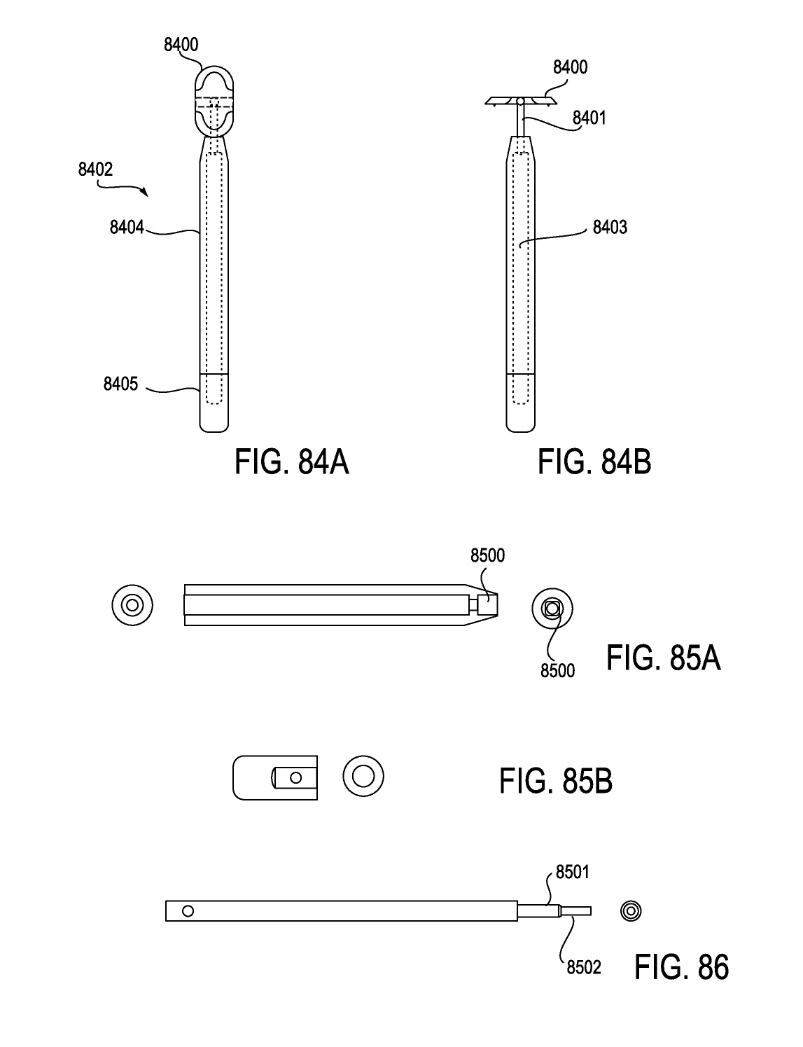

FIGS. 84A-86 show an exemplary embodiment of a device (similar to the device described in above, for example) and handle 8402 used for delivering a device and performing an interspinous fusion unilaterally. As shown in FIGS. 84A-86, the devices and systems may include a first fixation plate 8400 and a rod 8401. The rod may be threaded along its length, or a portion thereof. The rod may have a non circular cross section (e.g. rectangular, as shown), such that the handle 8402 can be used to manipulate the rod and device without rotating about the rod. Furthermore, as described above, the barrel will not rotate about the rod. The rod may also be hollow along at least a portion of the length. The rod may include internal threads in addition to external threads.

As shown in FIGS. 84A and 84B, the device and system may also include a handle configured to couple to the rod and first fixation plate. The handle may be used to angle the fixation plate with respect to the rod, fix the angle of the plate with respect to the rod, rotate the device, position and implant the device, and/or any combination thereof. As shown, the handle includes an internal handle portion 8403 and an external handle portion 8404, and in some embodiments an end cap 8405 that may function to couple the internal portion to the external portion. The external handle portion may be sized and configured to couple to the outer portion of the rod. For example, as shown in FIG. 85A, the external handle may have a rectangular shaped recess 8500 at the distal end of the handle, sized and configured to receive the rectangular cross section of the rod. In some embodiments, because the external portion does not thread onto the rod, the external portion may be rotated (to rotate the device) without removing the handle (e.g. internal handle portion) from the device.

The internal handle portion may be sized and configured to couple to an internal portion (e.g. the internal threads) of the rod. For example, as shown in FIG. 86, the internal handle may have a threaded portion 8501 toward the distal end of the internal handle, sized and configured to thread into the rod and couple to the internal threads of the rod. Furthermore, as shown, the internal handle portion may include a locking portion 8502. This extension of the internal portion at the distal end may function to press onto the joint between the plate and the rod and lock the joint in position. For example, the plate may be rotated with respect to the rod such that they are positioned at a 45 degree angle to one another. The locking portion may then be placed against the joint, such that the joint can no longer rotate, and the plate is locked in position with respect to the rod. In some embodiments, the locking portion will be placed against the rod by advancing (e.g. threading) the internal handle portion further into the rod until the locking portion is touching the joint. To release the locking portion and the joint, the internal handle portion may be pulled out of (e.g. un-threaded from) the rod. As shown, in some embodiments, the internal handle portion may be configured to receive a locking pin at its proximal end. This hole in the proximal end of the internal portion may be lined up with a hole in the end portion, as shown in FIG. 85B. The hole in the end portion may also be sized and configured to receive a locking pin.

The handle as described herein may be used for performing a unilateral spinal fusion. An exemplary method may include the step of deploying a first fixation plate from a first lateral side of a spinous process to the opposite lateral side of the spinous process. The rod may be threaded onto the internal handle portion and then the external handle portion may be placed over the internal handle portion and over the external portion of the rod. The first fixation plate may rotate with respect to the rod and then the internal handle portion may be rotated/threaded into the rod such that the locking portion fixes the joint and locks the plate with respect to the rod. The end cap may then be placed over the internal handle portion and may couple the internal handle portion to the external portion. Alternatively, the external handle portion may be coupled to the rod after the locking portion of the internal handle portion is in place. Once the handle is coupled to the rod, the handle may be used to position the device within a spine of a patient. The handle may be used to position the first fixation plate on the opposite lateral side of the spinous process from a first lateral side of a spinous process.

In some embodiments, once the first fixation plate is in position in a spine, the rod may be rotated with respect to the plate such that they form about a 90 degree angle. The internal handle portion may then be used to lock the plate and rod into this position. The barrel, as described above, may then be deployed over the handle such that it couples to the first fixation plate. Once the barrel is coupled to (and/or locked onto) the first fixation plate, the handle may be removed. Alternatively, the handle may not be removed until the first fixation plate is coupled to the barrel, rod, and/or first fixation plate.

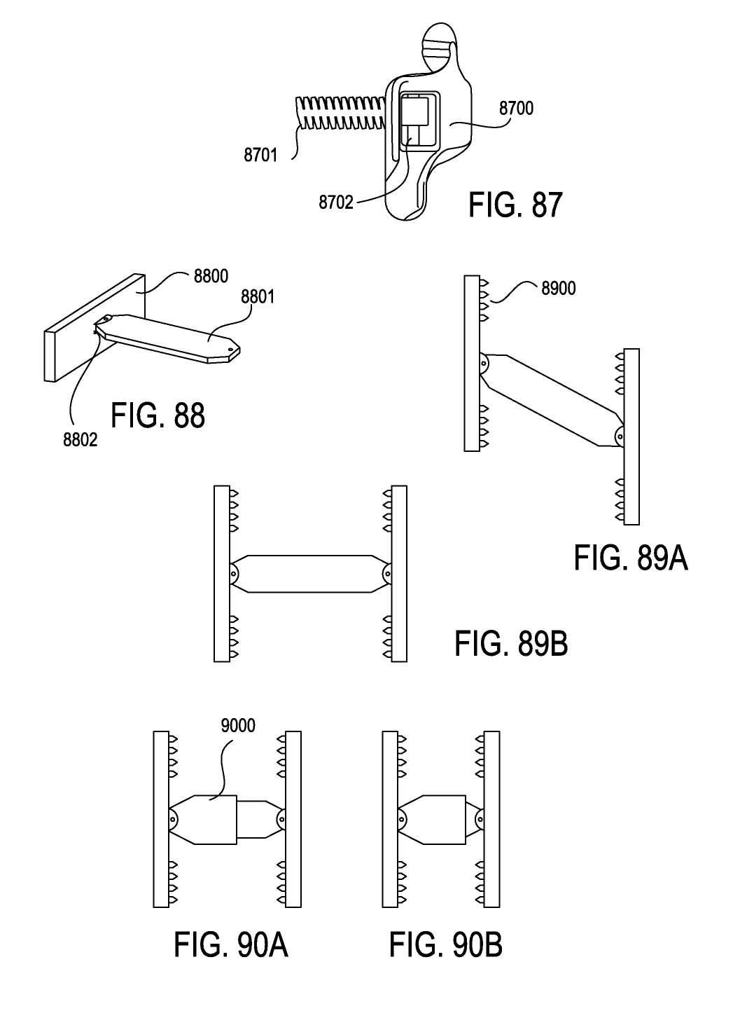

FIG. 87 shows an exemplary alternative embodiment of an interspinous fusion device, specifically one that may be deployable unilaterally. As shown, the devices and systems may include a first fixation plate 8700 having a rod 8701. The first fixation plate may be coupled to a rod via a joint 8702 such that the plate is slideable (and/or pivotable) with respect to the rod about the joint. As shown, the rod may slide with respect to the first fixation plate such that the plate and rod may be delivered and implanted in a method as described above.

FIGS. 88-90B show an alternative embodiment of an interspinous fusion device, specifically one that may be deployable unilaterally. This device and method may be similar to the devices described above in that the fixation plate(s) of this embodiment are rotatable with respect to the rod. As shown in FIG. 88, the device of this embodiment includes a fixation plate 8800 coupled to a rod 8801 via a joint 8802 such that the plate is pivotable with respect to the rod about the joint. The fixation plate and rod may be provided to a user already connected, or alternatively, a user may connect the fixation plate to the rod. As shown in FIGS. 89A and 89B, the device may include a first fixation plate, a second fixation plate, and a rod pivotably connected to the two fixation plates. As shown, the fixation plate(s) may include coupling members 8900 positioned on the back side of the fixation plate(s) (i.e. the side facing the spinous processes while implanted). As described above, the coupling members may be spikes or protrusions sized and configured to dig into or catch onto bone thereby aiding in the coupling of the fixation plate to the spinous process(es). In some embodiments, the spikes may be biased such that they may slide along the bone of the spinous process while being moved in a first direction, and then in bite into the bone when moved in a second direction and/or compressed against/into the bone. As shown in FIG. 89B, each of the fixation plates may be rotated such that the width (left to right in the figure) of the device is reduced, and the device may be implanted between the spinous processes. In some embodiments, only one of the plates may be rotated with respect to the rod. For example, the plate that is coupled to the opposite lateral side of the spinous processes may be rotated to fit between the spinous processed. Once the plate is deployed beyond the spinous processes, one or both of the plates may be rotated such that they couple to the first lateral side and the opposite lateral side of the spinous processes. As shown in FIG. 90A, the rod coupling the two fixation plates may have an adjustable length. For example, once the device is implanted, and the plates are on the two opposite sides of the spinous processes, the plates may be compressed against the spinous processes by reducing the length of the rod. As shown, the rod 9000 may be telescopic such that a first portion of the rod may be pushed into a second portion of the rod. The length of the rod may be reduced (or increased) by any other suitable mechanism.

In some embodiments, the device may be deployed bilaterally. For example, as described above, the method for performing a bilateral spinal fusion may include the step of deploying the second fixation plate from the opposite lateral side of the spinous process to position the plate against the opposite lateral side of the spinous process. In this embodiment, the plate may be coupled to the rod, and the rod may be positioned such that it is perpendicular to the fixation plate. Alternatively, the rod may not be coupled to the plate while the plate is implanted or the rod may be positioned with respect to the plate at any suitable angle. Once the plate and the rod are in position, the first fixation plate may be coupled to the rod (or adjustable rod) from the first lateral side of the spinous process to position the plate against the first lateral side of the spinous process.

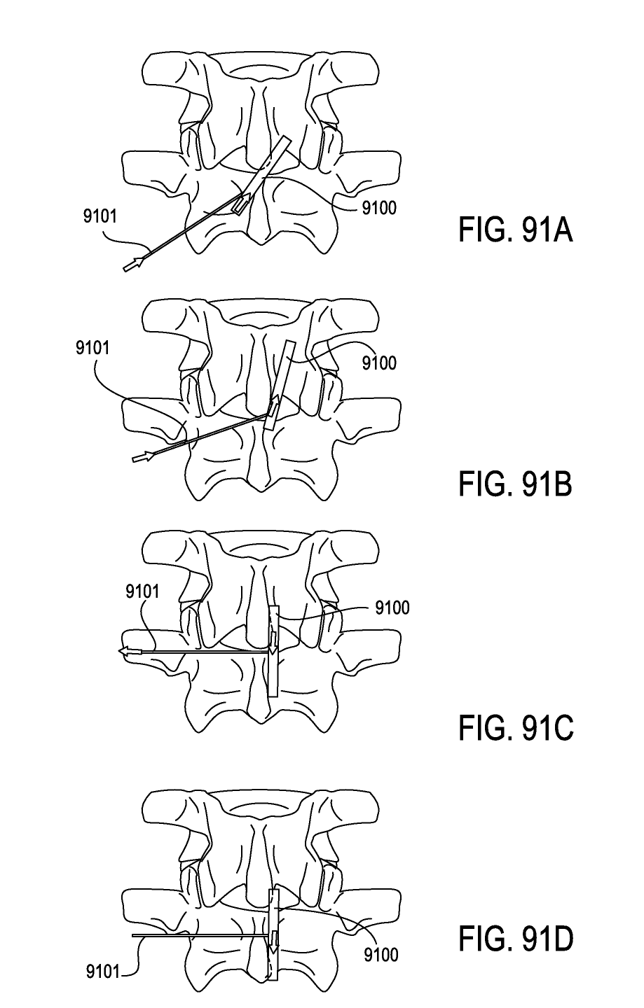

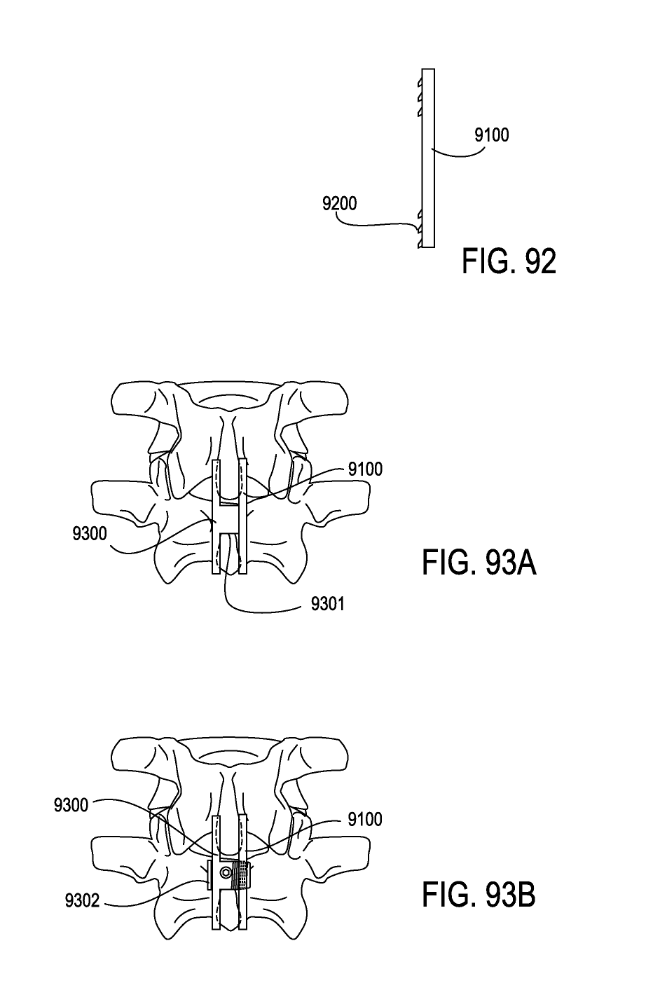

FIGS. 91A-92 show an alternative embodiment of an interspinous fusion device, specifically one that may be deployable unilaterally (and/or bilaterally as described above). This device and method may be similar to the devices described above in that the fixation plate(s) of this embodiment are rotatable with respect to an implantation tool. As shown in FIG. 91A, the device of this embodiment includes a fixation plate 9100 removably coupled to an insertion tool 9101 via a joint such that the plate is pivotable with respect to the tool about the joint. The fixation plate and tool may be provided to a user already connected, or alternatively, a user may connect the tool to the fixation plate. As shown in FIGS. 91A-91D, the fixation plate may be deployed from a first lateral side of the spinous processes to the opposite lateral side using the insertion tool. Similar to the method described above, the fixation plate may be inserted between the spinous processes from cephalad aspect of spine, at an angle to the length of the spine (which runs left to right in FIGS. 91A-91D). As the plate is pushed by the insertion tool between the spinous processes and against the opposite side of the caudal (left) spinous process, the tool may rotate with respect to the plate. Once the plate is between the two adjacent spinous processes, the tool may be pushed inward (up in the figures) to push the trailing end of the plate between and behind the spinous processes, as shown in FIG. 91C. Once the plate is adjacent to the opposite lateral sides of the spinous processes, the tool may be pulled in the cephalad (right) direction to center the plate behind the two adjacent spinous processes. Once in position, the tool may be removed from the fixation plate. Alternatively, the second fixation plate 9300 (as shown in FIGS. 93A and 93B) may be coupled to the first fixation plate 9100 over the insertion tool, and then the tool may be removed. As shown in FIG. 92, the fixation plate may include coupling members 9200 positioned on the back side of the fixation plate (i.e. the side facing the spinous processes while implanted). As described above, the coupling members may be spikes or protrusions sized and configured to dig into or catch onto bone thereby aiding in the coupling of the fixation plate to the spinous process(es). As shown, the spikes may be biased such that they may slide along the bone of the spinous process while being moved in a first direction (e.g. caudal—left in the figures), and then in bite into the bone when moved in a second direction (e.g. cephalad—right in the figures) and then compressed against/into the bone.

FIGS. 93A-93B show an exemplary embodiment of an interspinous fusion fixation device, specifically one that comprises the fixation plate of FIGS. 91A-92. As shown, the device further includes a second fixation plate 9300 that couples to the first lateral side of the spinous processes and couples to the fixation plate as described above with reference to FIGS. 91A-92. In some embodiments, as shown in FIGS. 93A and 93B, the fixation plate 9300 may further include a post 9301. The post may be coupled to the back side of the fixation plate (i.e. the side facing the spinous processes while implanted) and may be sized and configured to be positioned between two adjacent spinous processes. In some embodiments, the post may further function to distract the two spinous processes. The post may also be sized and configured to receive a graft material. The graft material may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the post may be filled with a filer material such as bone cement. The post may be configured such that is has a window or windows through which the bone graft may contact the spinous processes and, in some instances, promote fusion. In some embodiments, the spinous processes may be decorticated prior to the implantation of the fixation plate and/or bone graft to also, in some instances, promote fusion. As shown in FIG. 93B, the device may further include a locking mechanism 9302 to couple the two fixation plates to one another. The locking mechanism may also function to compress the fixation plates against one another. As shown, the locking mechanism may be fed through the second fixation plate 9300, through the post, and then into the first fixation plate 9100. As shown, the locking mechanism may thread into threads on an inner diameter of the first fixation plate, while the locking mechanism may freely rotate within the second fixation plate. As the locking mechanism is rotated, it will pull the first fixation plate back toward and into the opposite side of the spinous processes. A lip on the locking mechanism, as shown, will push the second fixation plate into the first lateral side of the spinous processes. Once both the first and second fixation plates are compressed against/into the lateral sides of the spinous processes, a set screw may tighten down the locking mechanism and/or fixation plate into place, as shown.

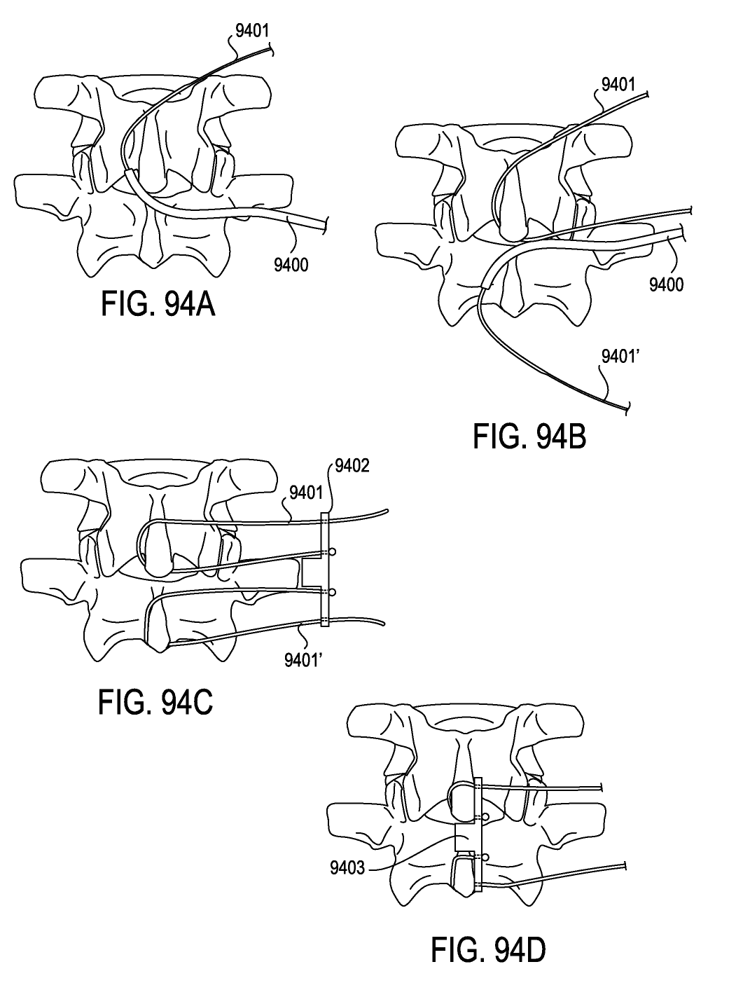

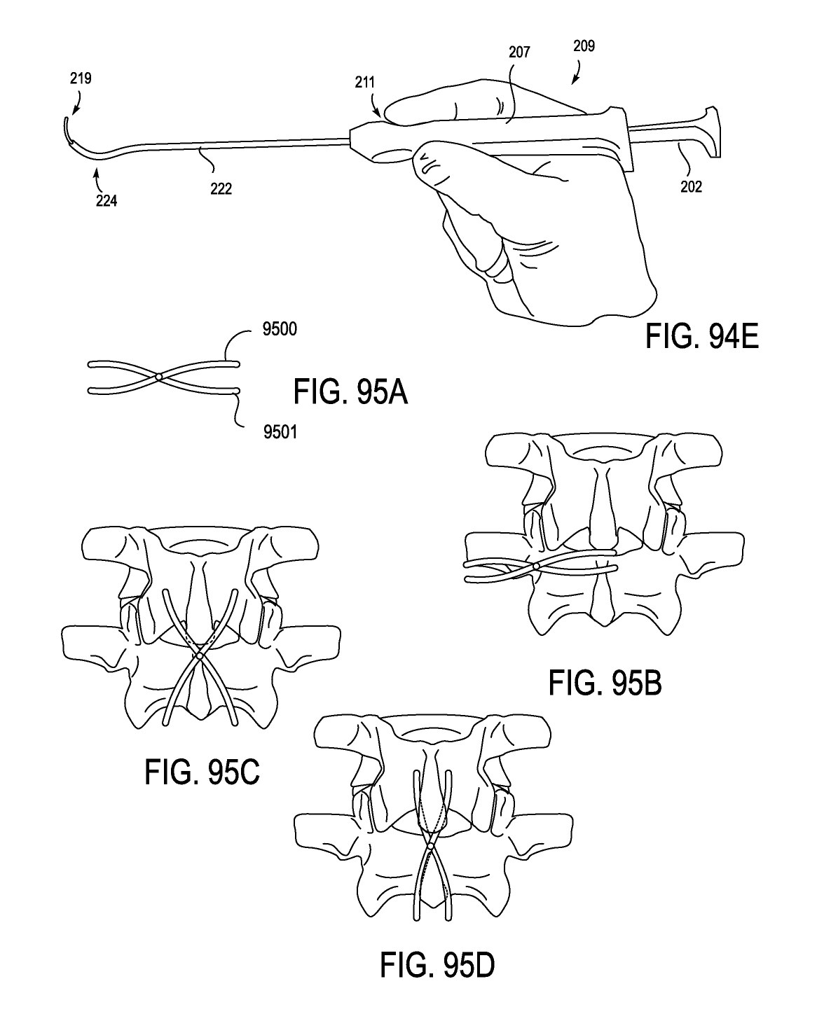

FIGS. 94A-94D show an exemplary embodiment of an interspinous fusion device, specifically one that may be deployable unilaterally. As shown in FIG. 94A, a probe device 9400 may be delivered between two adjacent spinous processes from the first lateral side of the spinous processes to the opposite lateral side of the spinous processes. For example, as shown in FIG. 94E, the probe 209 includes a handle portion having a pusher 202 that communicates with an internal cannula 219 slideably disposed within the external cannula 224 so that the internal cannula may be extended from the distal end of the probe for placement around the spinous process, as illustrated in FIGS. 94A and 94B. The pusher includes a flanged proximal end having a finger (e.g., thumb) pushing surface that is perpendicular to the long axis of the device (including the long axis of the handle). This proximal end may be formed to more readily allow insertion of a wire by guiding the wire into the lumen of the inner cannula. The probe 209 may be held, pencil-like, with the fingertips grasping the waist region. The pusher 202 is shown partially pushed in, showing the distal end of the probe 219 extending partially from the distal end 224 of the outer cannula 222. The outer cannula of the probe is fixed relative to the handle region 207, and had a curved distal end 224. In this example, the curve is crescent-shaped, so that it initially curves away from the centerline of the probe (e.g., towards the ‘back’ of the probe), and then curves back towards the front of probe, as shown. The inner cannula 219 of the probe is pre-biased so that it assumes a curved shape upon leaving the outer cannula 222. For example, the inner cannula may be a Nitinol cannula having a pre-set curved shape. The cannula may be solid, woven, mesh, etc., but includes a passageway for the wire. The distal tip of the inner cannula may be configured so that is substantially atraumatic. It may also be configured so that it cannot be withdrawn into the cannula (e.g., it may have a slightly larger OD than the ID of the outer cannula, etc.). In some variations, the distal tip may be blunted or rounded. Alternatively, the distal tip may be configured to cut tissue and/or dissect away tissue (such as the paraspinous muscles) from the lateral sides of the spinous processes.

As shown in FIGS. 94A, once the outer cannula of the probe is between the spinous processes, the inner cannula of the probe may be deployed around the back side of one of the spinous processes. Once the probe is deployed, a wire 9401 may be fed through the probe such that it is deployed between the spinous processes, around the back side of one of the spinous processes, and then back toward the first lateral side of the spinous process. Once the first wire is in position, the probe may be removed. In some embodiments, once the probe is pulled back off of the deployed wire, the wire may expand along the length of the wire, or a portion thereof, such that a wider width coupled to the back side of the spinous process. Alternatively, the wire(s) may be used to pull in a fixation plate device to the opposite side of the spinous processes.

As shown in FIG. 94B, the steps described above may be repeated with a second wire 9401′ and the second spinous process. In some embodiments, the same probe may be used multiple times to deliver multiple wires. As shown in FIG. 94C, once the wires are deployed and/or the fixation plates have been pulled into position behind the spinous processes, a fixation plate 9402 may be coupled to the ends of the wires. The wires (and/or fixation plates) behind the spinous processes may be pulled tight against the back side of the spinous processes, while the fixation plate is pushed up against the first lateral side of the spinous processes. Once in position, the plate may be locked onto the wires to hold the device firmly against the sides of the spinous processes, as shown in FIG. 94D. As shown in FIGS. 94C and 94D, the fixation plate may further include a post 9403 as described above.

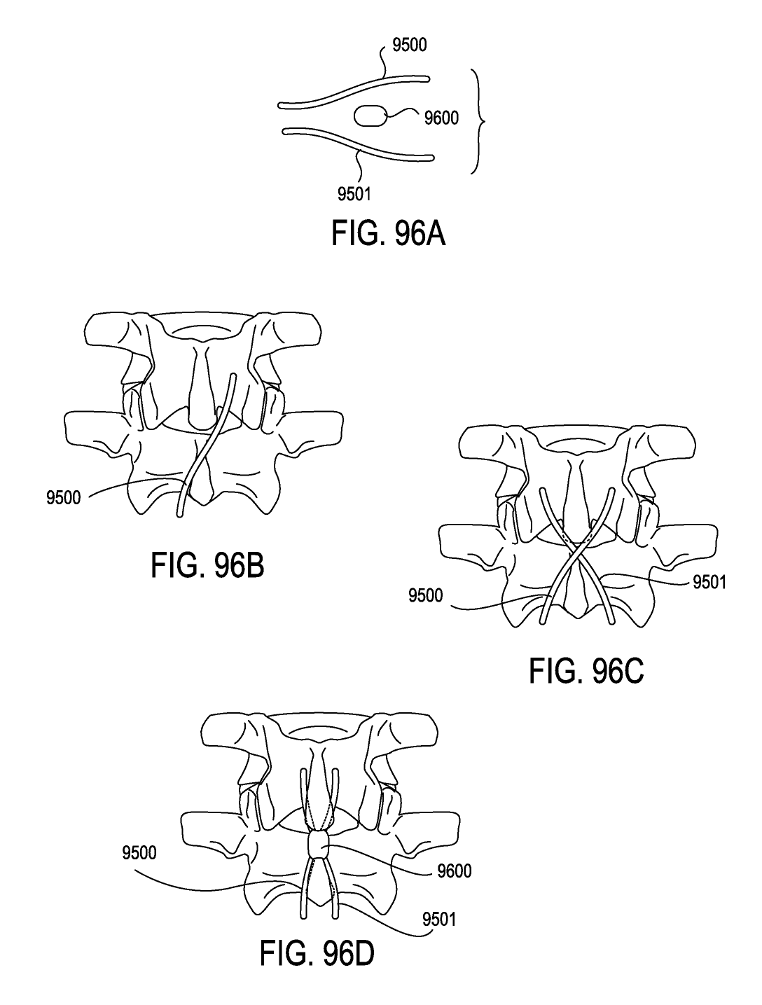

FIGS. 95A-95D and 96A-96D show exemplary embodiments of interspinous fusion devices, specifically ones that may be deployable unilaterally and include a first and second rotatable fixation plate 9500 and 9501. As shown, the fixation plates are roughly S-shaped, each having a first and second curved portion that are each coupleable to a lateral surface of a first and second spinous process. As shown in FIG. 95A, the fixation plates may be coupled to one another such that they form an X-shaped device. The device may be deployed from a single side of the spinous processes. Once the device is beyond the spinous processes, the fixation plates may be rotated, as shown in FIG. 95C, such that the curved portions are moved against the lateral sides of the two spinous processes, as shown in FIG. 95D. As shown in FIG. 96A, the S-shaped fixation plates may be implanted one at a time. For example as shown in FIG. 96B, a first S-shaped fixation plate may be deployed toward the opposite side of the cephalad spinous process from the first side of the caudal spinous process. As shown in FIG. 96C, a second S-shaped fixation plate may be deployed toward the opposite side of the caudal spinous process from the first side of the caudal spinous process. In some embodiments, once the plates are implanted, they may be rotated such that they are pushed/pulled against the sides of the spinous processes. The first and second S-shaped fixation plates may be coupled to one another in any suitable fashion. In some embodiments, the coupling member 9600 may function to compress the fixation plates against the spinous processes.

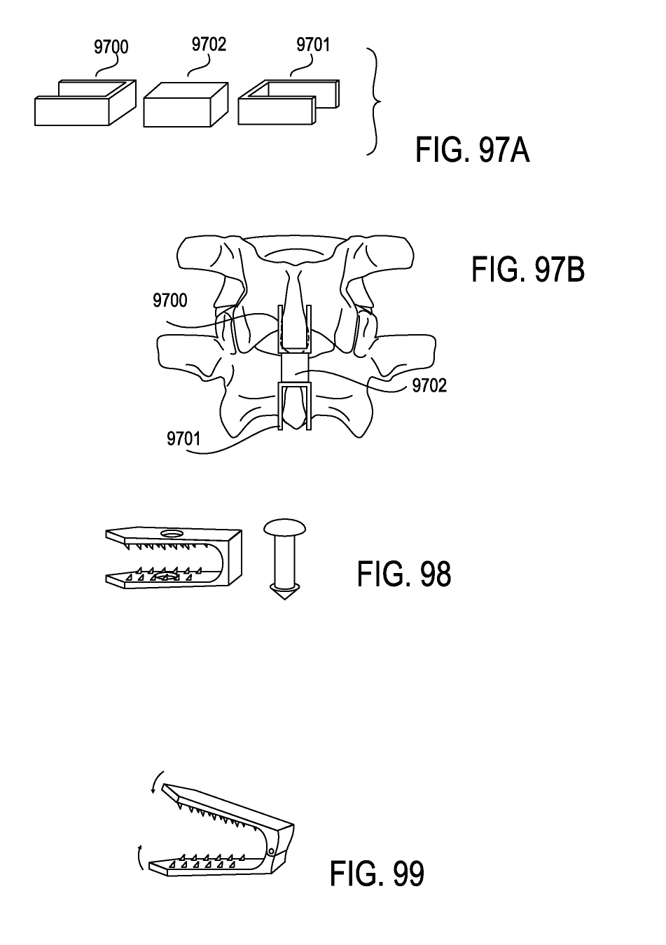

FIGS. 97A-99 show exemplary embodiments of interspinous fusion devices, specifically ones that may be deployable unilaterally and include a first and second fixation bracket. As shown in FIG. 97A, the device is shown in an exploded view. As shown, the device includes two fixation brackets 9700 and 9701. The brackets are sized and configured such that they may be positioned in a non-deployed mode, wherein the brackets are nestled within one another. In this configuration, the device is sized and configured such that it can be deployed between two spinous processes from a first lateral side. As shown in FIG. 97B, once the device is deployed between the spinous processes, the fixation brackets may be moved toward the spinous processes. Once may be moved caudal, while the other is moved cephalad such that each bracket is coupled to a spinous process. Although not shown, the device may include an element that couples the first and second brackets once in the deployed configuration. As shown in FIG. 97B, once the brackets are in position, a barrel 9702 may be coupled between the brackets. The barrel may function to hold the brackets in plate, to distract the spinous processes, and/or to provide a location to hold a bone graft material. The barrel may be configured such that is has a window or windows through which, the bone graft may contact the spinous processes and, in some instances, promote fusion. In some embodiments, the brackets may also include windows that are in line with the windows of the barrel such that the bone graft may contact the spinous processes through the barrel and through the brackets. FIG. 98 illustrates a device having fixation brackets as described above, however, as shown in FIG. 99, the bracket may have a roughly V-shaped geometry and may include coupling members (e.g. spikes). Once the brackets are moved adjacent to the spinous processes, the V-shape may be compressed against the spinous process such that the coupling members may bite into the bone, for example. As described above, the spikes may be biased such that they may slide along the bone of the spinous process while being moved in a first direction (e.g. as the bracket is moved from between the spinous processes to adjacent to a spinous process), and then in bite into the bone when moved in a second direction and/or compressed against/into the bone. As shown in FIG. 98, once the bracket is in position and/or compressed against the bone, a fastening screw (or other mechanism) may be coupled to the spinous processes and the brackets to fix the device into place.

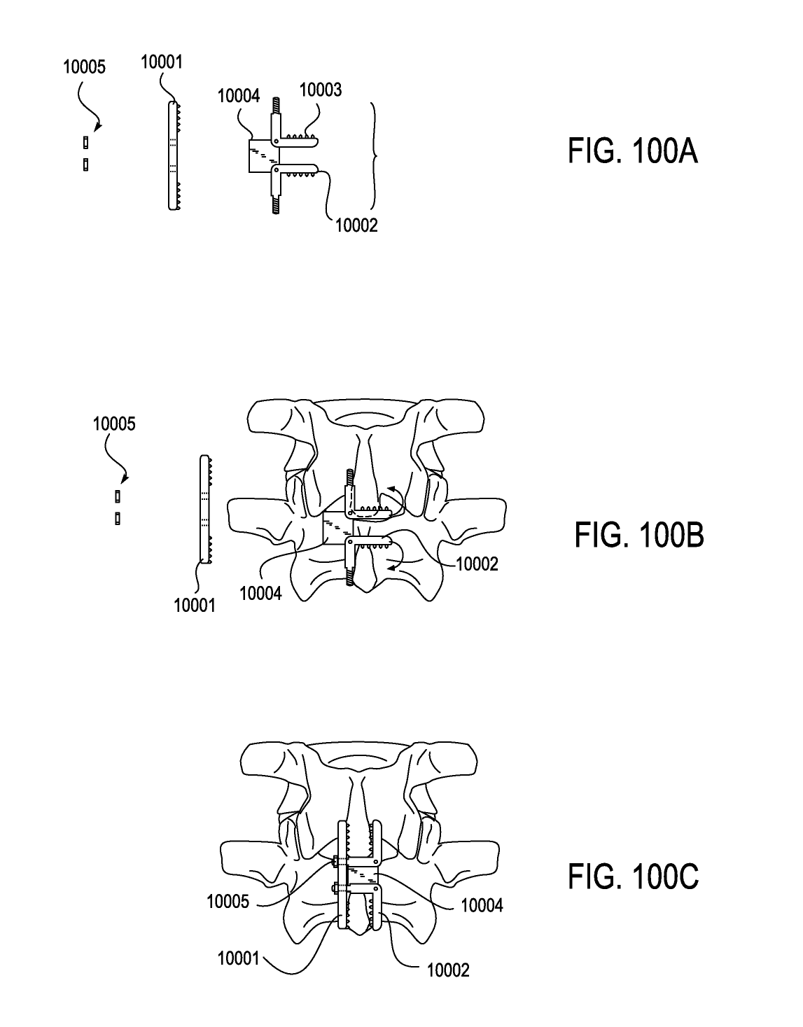

FIGS. 100A-100C show an exemplary embodiment of an interspinous fusion device, specifically one that may be deployable unilaterally. As shown, the devices and systems may include a fixation plate 10001 sized and configured to couple to a fist lateral side of a spinous process and a coupling backing or backings, sized and configured to couple to the opposite lateral side of the spinous process. The coupling backings 10002 may be an L-shaped bracket having two legs. The first leg may be sized and configured to couple to the opposite lateral side of the spinous process. In some embodiments, the first let may include coupling members 10003 (e.g. spikes) as described above.

As shown, the coupling backing(s) may be coupled to a post 10004 sized and configured to be positioned between two adjacent spinous processes. The brackets may be coupled to the post such that they may rotate with respect to the post. In some embodiments, the post may further function to distract the two spinous processes. The post may also be sized and configured to receive a graft material and may be configured such that is has a window or windows through which, the bone graft may contact the spinous processes and, in some instances, promote fusion. As shown, the fixation plate may also be coupled to the post.

As shown in FIG. 100A, as the device is deployed between the spinous processes, the second leg of the coupling backing may hit the first lateral side of the spinous process, such that the L-shaped bracket is rotated and the first leg of the coupling backing is rotated toward the opposite lateral side of the spinous process, such that it couples to the opposite lateral side of the spinous process, as shown in FIG. 100B. Once the device is in place such that the post is between the spinous processes and the first leg of the L-shaped bracket is coupled to the opposite lateral side of the spinous process, the fixation plate 10001 may be slid over the post toward the first lateral side of the spinous process. The fixation plate may also include coupling members such as spikes, such that it is coupled to the first lateral side of the spinous process. As shown, the fixation plate may also be slid over the second leg(s) of the coupling backing and may lock them into place such that they cannot rotate with respect to the post. Once the device is in position a set screw 10005 or other locking mechanism may optionally be locked down to fix the device into position.

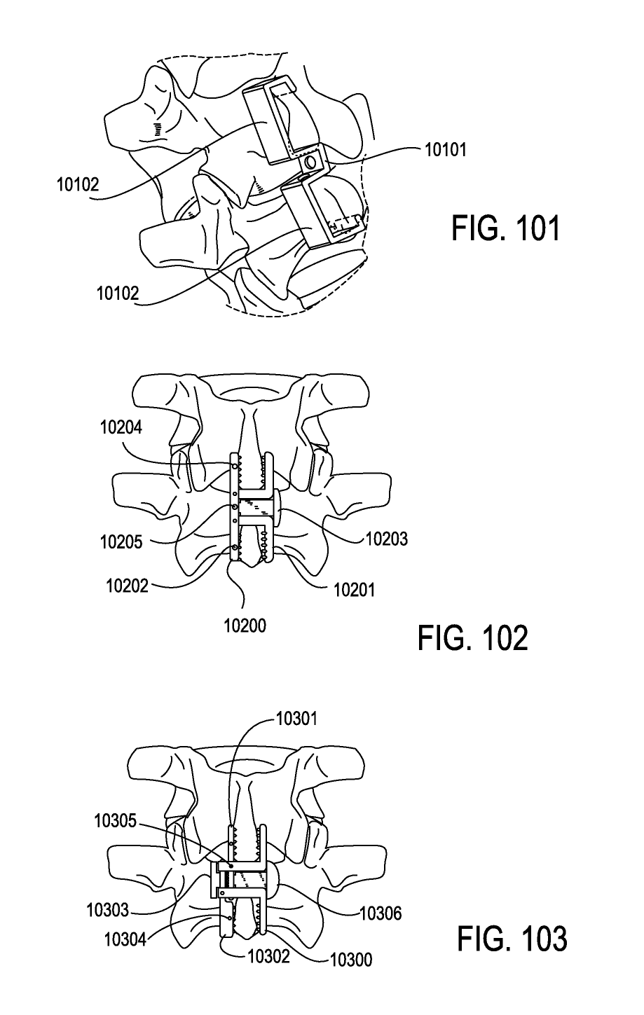

FIG. 101 shows an exemplary embodiment of an interspinous fusion device, specifically one that may be deployable unilaterally. As shown in the top view of FIG. 101, the device of this embodiment may include a fixation plate having a center post region 10101 and two adjustable wings 10102. As shown in FIG. 101, the adjustable wings may each have a square or rectangular shaped geometry, such that the wing has a portion that is sized and configured to coupled to the opposite lateral side of the spinous process. As shown in FIG. 101, the device may be deployed from the first lateral side of the spinous processes. The post may be placed between a second and third adjacent spinous process, while the cephalad (right) wing may be placed between a first cephalad spinous process (not shown) and the second spinous process and the caudal (left) wing may be placed between the third spinous process and a fourth caudal spinous process (also not shown). Once the device is in place, the cephalad and caudal wings may be moved toward the post such that they wrap around the cephalad aspect of the second spinous process and the caudal aspect of the third spinous process, respectively, as shown in FIG. 101. As described throughout, the post may be sized and configured to receive graft material.

FIG. 102 shows a top view of an exemplary embodiment of an interspinous fusion device, specifically one that includes at least one paddle and that may be adjustable in at least the cephalad/caudad direction. As shown, the devices and systems may include an adjustable fixation plate 10200 including, as shown, a caudal portion and a cephalad portion; and a coupling backing 10201, sized and configured to couple to the opposite lateral side of the spinous process. In this embodiment, the coupling backing comprises a paddle. In some embodiments, as shown, the coupling backing comprises two paddles; one coupled to each of the two adjacent the spinous processes on opposite lateral side of the spinous processes. As shown, the fixation plate may be positioned within the spine such that it is coupled to two adjacent spinous processes. The fixation plate may be positioned on a single lateral side of the spinous processes. The fixation plate may be attached to both spinous processes to stabilize the segment of the spine and promote fusion. As shown, the fixation plate 10200 may include a caudal portion and a cephalad portion that are slideable within one another. For example, the caudal portion may couple to the caudal spinous processes, while the cephalad portion may couple to the cephalad spinous process. The caudal portion may be coupled to the cephalad portion toward the center of the device with a locking mechanism. This embodiment may provide an advantage that it may be adjusted to fit varying patient anatomy. For example, in some patients two adjacent spinous processes may be located further from one another than in other patients—the caudal portion and the cephalad portion may be expanded with respect to one another, i.e. the width is expanded. Additionally, this embodiment may function to distract the spinous processes by coupling the inner fixation plate to the outer fixation plate after the spinous processes have be distracted. Alternatively, this embodiment may function to compress the spinous processes toward one another—the caudal portion and the cephalad portion may be moved toward one another, i.e. the width is reduced. In some embodiments, the compression of the spinous processes against the post may promote fusion. Especially in some cases if the post includes a graft material and/or the spinous processes have been roughened or decorticated prior to their compression.

The fixation plate may be coupled to the paddle(s) by way of set screws 10202, for example. The screws may be fed through holes in the fixation plate and then into each of the paddles. The screws may be deployed into the fixation plate from the single lateral side of the spinous processes—e.g. through the same incision as the fixation plate. In some alternative variations, the screws may be dowels, for example.

In some embodiments, as shown in FIG. 102, a portion of the fixation plate (e.g. the caudal portion) may further include a post 10203. The post may be coupled to the back side of the fixation plate (i.e. the side facing the spinous processes while implanted) via a locking mechanism 10205 and may be sized and configured to be positioned between two adjacent spinous processes. In some embodiments, the post may further function to distract the two spinous processes. The post may also be sized and configured to receive a graft material. As shown in FIG. 102, in some embodiments, the fixation may include a plurality of holes 10204. These holes may be sized and configured to receive a tool. In some embodiments the tool may be configured to move (e.g. slide) the caudal portion and the cephalad portion with respect to one another in order to distract and/or compress the spinous processes before, during, and/or after the implantation of the interspinous process device.

FIG. 103 shows a top view of an alternative exemplary embodiment of an interspinous fusion device, specifically one that includes at least one paddle 10300 and that may be adjustable in at least the cephalad/caudad direction. As shown, the devices and systems may include a fixation plate including an inner plate 10301 and an outer plate 10302; and a coupling backing, sized and configured to couple to the opposite lateral side of the spinous process. In this embodiment, the coupling backing comprises a paddle 10300. In some embodiments, as shown, the coupling backing comprises two paddles; one coupled to each of the two adjacent the spinous processes on opposite lateral side of the spinous processes. As shown, the fixation plate may include an inner fixation plate and an outer fixation plate. For example, the inner fixation plate may couple to the caudal (left) spinous processes, while the outer fixation plate may couple to the cephalad (right) spinous process. As shown, a portion of the inner fixation plate may also couple to the cephalad (right) spinous process. The inner fixation plate may be coupled to the outer fixation plate toward the center of the device with a locking mechanism 10303. This embodiment may provide an advantage that it may be adjusted to fit varying patient anatomy. For example, in some patients two adjacent spinous processes may be located further from one another than in other patients. Additionally, this embodiment may function to distract the spinous processes by coupling the inner fixation plate to the outer fixation plate after the spinous processes have be distracted. Alternatively, this embodiment may function to compress the spinous processes toward one another. In some embodiments, the compression of the spinous processes against the post may promote fusion. Especially in some cases if the post includes a graft material and/or the spinous processes have been roughened or decorticated prior to their compression.

In some embodiments, the locking mechanism 10303 may function to move the inner plate and the outer plate with respect to one another. For example, the locking mechanism and the fixation plates may have a rack and pinion configuration such that the locking mechanism may be rotated such that it pulls at least one of the inner plate and the outer plate toward the center of the device, thereby compressing the spinous processes toward one another and/or toward a post. As shown in FIG. 103, in some embodiments, the fixation plate may include a plurality of holes 10304. These holes may be sized and configured to receive a tool. In some embodiments the tool may be configured to distract and/or compress the spinous processes before, during, and/or after the implantation of the interspinous process device. In some embodiments, the fixation plate and the paddle may have holes that line up such that a set screw or other locking mechanism 10305 may couple the paddle to the fixation plate. In some embodiments, the device may further include a post 10306.

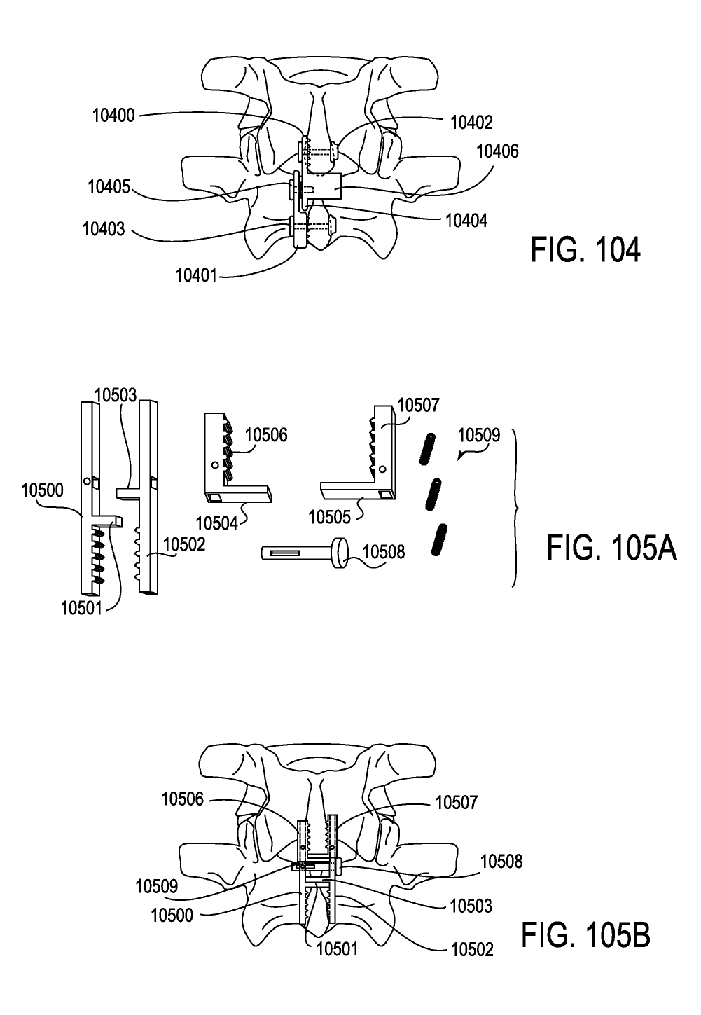

FIG. 104 shows an exemplary embodiment of an interspinous fusion device, specifically one that may be deployable unilaterally. As shown, the devices and systems may include a fixation plate including an inner plate 10400 and an outer plate 10401; a coupling screw 10403, sized and configured to couple the fixation plate(s) to a fist lateral side of a spinous process; and a coupling backing 10402, sized and configured to couple to the opposite lateral side of the spinous process. As shown, the fixation plate may be positioned within the spine such that it is coupled to two adjacent spinous processes. The fixation plate may be positioned on a single lateral side of the spinous processes. The fixation plate may be attached to both spinous processes to stabilize the segment of the spine and promote fusion. As shown, the fixation plate may include an inner fixation plate and an outer fixation plate. For example, the inner fixation plate may couple to a first spinous processes, while the outer fixation plate may couple to a second spinous process. As shown, a portion 10404 of the inner fixation plate may also couple to the second spinous process. The inner fixation plate may be coupled to the outer fixation plate toward the center of the device with a locking mechanism 10405. This embodiment may provide an advantage that it may be adjusted to fit varying patient anatomy. For example, in some patients two adjacent spinous processes may be located further from one another than in other patients. Additionally, this embodiment may function to distract the spinous processes by coupling the inner fixation plate to the outer fixation plate after the spinous processes have be distracted. Alternatively, this embodiment may function to compress the spinous processes toward one another. In some embodiments, the compression of the spinous processes against the post may promote fusion. Especially in some cases if the post includes a graft material and/or the spinous processes have been roughened or decorticated prior to their compression.

The fixation plate may be coupled to the spinous processes by way of coupling screws for example. The screws may be fed through holes in the fixation plate and then into each of the spinous processes. The screws may be deployed into the fixation plate (and into the spine) from the single lateral side of the spinous processes—e.g. through the same incision as the fixation plate. In some alternative variations, the screws may be dowels or bone dowels, for example.

In some embodiments, as shown in FIG. 104, the fixation plate (e.g. the inner fixation plate) may further include a post 10406. The post may be coupled to the back side of the fixation plate (i.e. the side facing the spinous processes while implanted) and may be sized and configured to be positioned between two adjacent spinous processes. In some embodiments, the post may further function to distract the two spinous processes. The post may also be sized and configured to receive a graft material. In some embodiments, the locking mechanism may function to move the inner plate and the outer plate with respect to one another. For example, the locking mechanism and the fixation plates may have a rack and pinion configuration such that the locking mechanism may be rotated such that it pulls at least one of the inner plate and the outer plate toward the center of the device, thereby compressing the spinous processes toward one another and/or toward a post.

FIGS. 105A and 105B show an exemplary embodiment of an interspinous fusion device that may be adjustable in at least the cephalad/caudad direction. As shown, the device and system may include a first fixation plate 10500 including spacer portion 10501 and a second fixation plate 10502 also including a spacer portion 10503. The first fixation plate is sized and configured to couple to a fist lateral side of a spinous process while the second fixation plate is sized and configured to couple to the second opposite lateral side of a spinous process. The spacer portions of the plates are sized and configured to couple between two adjacent spinous processes. As shown in FIG. 105A, the spacer portions 10502 and 10503 couple to a first spinous process while spacer portions 10504 and 10505 (described below) couple to a second spinous process. Alternatively, any combination of spacer portions may couple to either of the two adjacent spinous processes. The spacer portions are sized and configured to distribute and minimize load from the spinous processes. Also shown in FIGS. 105A and 105B, each of the fixation plates includes a slider portion. For example, the first fixation plate 10500 includes the slider portion 10506 and the second fixation plate 10502 includes a slider portion 10507. As shown, the slider portion 10506 is sized and configured to couple to a fist lateral side of a first spinous process while the slider portion 10507 is sized and configured to couple to the second opposite lateral side of the first spinous process.

One exemplary method of using the device as described in reference to FIGS. 105A and 105B includes the steps of implanting the first fixation plate 10500 with slider portion 10506 adjacent to the first lateral side of two adjacent spinous processes such that the spacer portions are between the spinous processes; implanting the second fixation plate 10502 and slider portion 10507 adjacent to the second opposite lateral side of two adjacent spinous processes such that the spacer portions are between the spinous processes and couple to the spacers 10501 and 10504 of the first fixation plate 10500 with slider portion 10506, respectively. Once the fixation plates are in position, the coupling members (for example, as shown, the coupling members may be spikes or protrusions) dig into or catch onto bone thereby aiding in the coupling of the plates to the spinous process. In some embodiments, the method may also include the step of implanting a locking mechanism (screw 10508 as shown in FIGS. 105A and 105B) that couples the first and second fixation plate. The screw may be locked in position by at least one set screw 10509. In some embodiments, the sliding portions may be slid along the fixation plates, respectively. In some embodiments, the sliding portions may move the spinous process toward the second spinous process such that the spine (or spinal level) is placed in compression. In some embodiments, the compression of the spinous processes against the spacer portions may promote fusion. Especially in some cases if the spacer portions include a graft material and/or the spinous processes have been roughened or decorticated prior to their compression. Alternatively, the sliding portions may move the spinous process away from the second spinous process such that the spine (or spinal level) is distracted. In alternative embodiments, the slider portions may both be coupled to and/or move the caudal spinous process, the slider portions may both be coupled to and/or move the cephalad spinous process, or the slider portions may be coupled to and/or move different spinous processes. Once the slider portions and/or spinous processes are positioned in the desired position, the slider portions may be fixed with respect to the fixation plates by way of set screws or any other suitable mechanism.

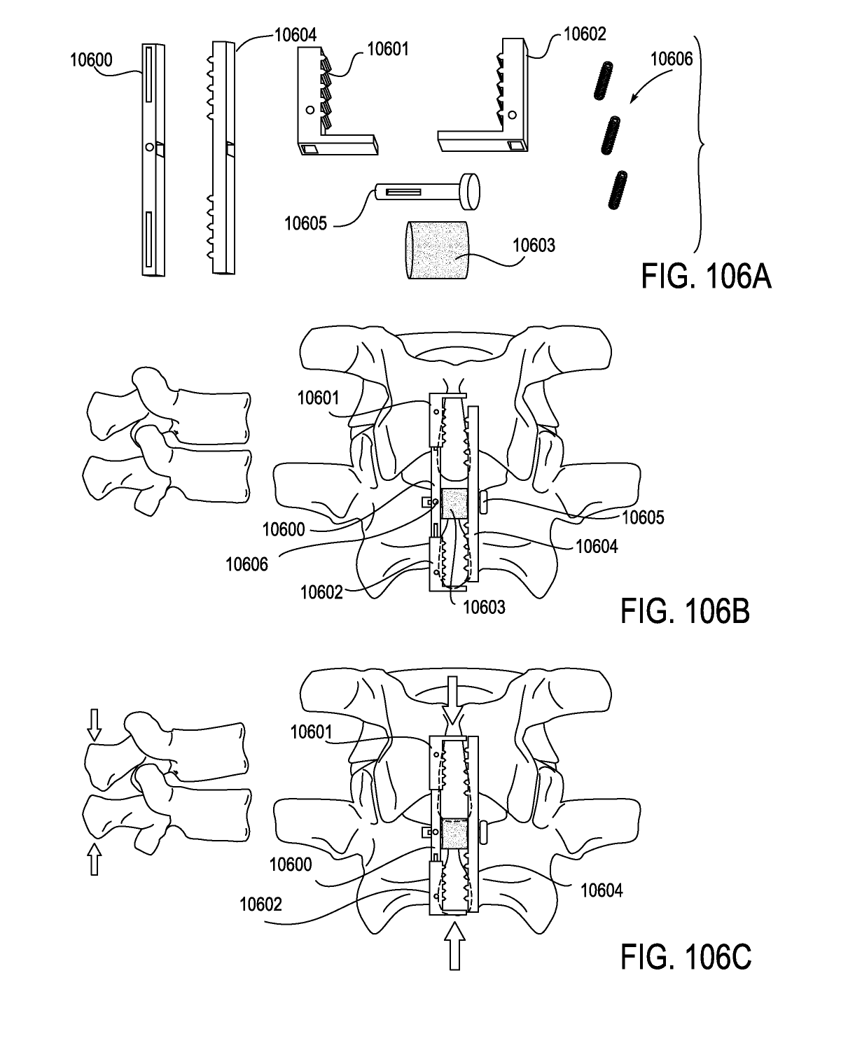

FIGS. 106A and 106B show an alternative exemplary embodiment of an interspinous fusion device that may be adjustable in at least the cephalad/caudad direction. This device and method may be similar to the devices described above in reference to FIGS. 105A and 105B with slider portions in an alternative configuration and the device may also include a spacer. One exemplary method of using the device as described in reference to FIGS. 106A and 106B includes the steps of implanting the first fixation plate 10600 (with first and second slider portions 10601 and 10602 and a spacer 10603) adjacent to the first lateral side of two adjacent spinous processes such that the spacer is between the spinous processes and implanting the second fixation plate 10604 adjacent to the second opposite lateral side of two adjacent spinous processes. Once the fixation plates are in position, the coupling members (for example, as shown, the coupling members may be spikes or protrusions) of the sliding members 10601 and 10602 may dig into or catch onto bone thereby aiding in the coupling of the plates to the spinous process. As shown in FIG. 106A, initially, the coupling members of the second fixation plate 10604 may not yet be engaged. In some embodiments, the method may also include the step of implanting a locking mechanism (screw 10605 as shown in FIGS. 106A and 106B) that couples the fixation plates. The screw may be locked in position by at least one set screw 10606. In some embodiments, as shown in FIG. 106C, the sliding portions 10601 and 10602 may be slid along the first fixation plate 10600 such that at least one of the spinous processes are moved toward one another such that the spine (or spinal level) is placed in compression. In some embodiments, the compression of the spinous processes against the spacer may promote fusion. Especially in some cases if the spacer includes a graft material and/or the spinous processes have been roughened or decorticated prior to their compression. Once the slider portions and/or spinous processes are positioned in the desired position, the slider portions may be fixed with respect to the fixation plates by way of set screws or any other suitable mechanism.

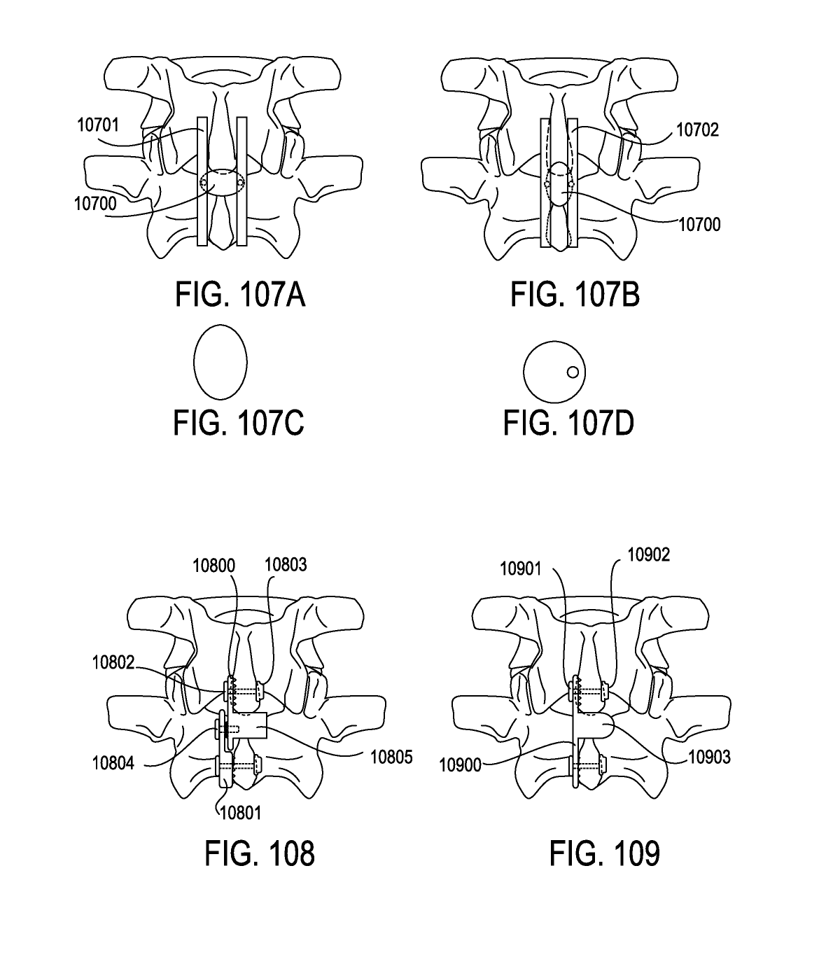

FIGS. 107A to 107D show an exemplary embodiment of an interspinous fusion device having an adjustable spacer 10700. In this embodiment, the device and system may include a first fixation plate 10701, a second fixation plate 10702, and an adjustable spacer positioned and/or coupled between the first and second plate. The first fixation plate is sized and configured to couple to a fist lateral side of a spinous process while the second fixation plate is sized and configured to couple to the second opposite lateral side of a spinous process. The spacer is sized and configured to be positioned between two adjacent spinous processes and moved from a non deployed configuration (107A) to a deployed configuration (107B). In the deployed configuration the spacer is coupled to both of the two adjacent spinous processes and functions to distribute and minimize load from the spinous processes and/or to promote fusion. As shown in FIG. 107C, the spacer may have a non circular shape, such as an elliptical or oval shape. Alternatively, as shown in FIG. 107D, the spacer may have a circular shape, but may be rotated off axis (e.g. a cam mechanism).

FIG. 108 shows an exemplary embodiment of an interspinous fusion device, specifically one that may be deployable unilaterally. As shown, the devices and systems may include a fixation plate including an inner plate 10800 and an outer plate 10801; a coupling screw 10802, sized and configured to couple the fixation plate(s) to a fist lateral side of a spinous process; and a coupling backing 10803, sized and configured to couple to the opposite lateral side of the spinous process. As shown, the fixation plate may be positioned within the spine such that it is coupled to two adjacent spinous processes. The fixation plate may be positioned on a single lateral side of the spinous processes. The fixation plate may be attached to both spinous processes to stabilize the segment of the spine and promote fusion. As shown, the fixation plate may include an inner fixation plate and an outer fixation plate. For example, the inner fixation plate may coupled to a first spinous processes, while the outer fixation plate may couple to the adjacent spinous process. As shown, a portion of the inner fixation plate may also couple to the adjacent spinous process. The inner fixation plate may be coupled to the outer fixation plate toward the center of the device with a locking mechanism 10804. This embodiment may provide an advantage that it may be adjusted to fit varying patient anatomy. For example, in some patients two adjacent spinous processes may be located further from one another than in other patients. Additionally, this embodiment may function to distract the spinous processes by coupling the inner fixation plate to the outer fixation plate after the spinous processes have be distracted.

The fixation plate may be coupled to the spinous processes by way of coupling screws for example. The screws may be fed through holes in the fixation plate and then into each of the spinous processes. The screws may be deployed into the fixation plate (and into the spine) from the single lateral side of the spinous processes—e.g. through the same incision as the fixation plate. In some alternative variations, the screws may be dowels or bone dowels, for example. The fixation plate may be coupled to the spinous processes in any suitable location. For example, the fixation plate may be couple to the mid to posterior portion of the spinous processes. Alternatively, the fixation plate may be placed more ventrally (anterior), near the lamina, rather than mid-high on the spinous process. For example, although not shown, the fixation plate may be placed in the more anterior position thereby replacing the spacer.

In some embodiments, as shown in FIG. 108, the fixation plate (e.g. the inner fixation plate) may further include a post 10805. The post may be coupled to the back side of the fixation plate (i.e. the side facing the spinous processes while implanted) and may be sized and configured to be positioned between two adjacent spinous processes. In some embodiments, the post may further function to distract the two spinous processes. The post may also be sized and configured to receive a graft material.

FIG. 109 shows an exemplary embodiment of an interspinous fusion device, specifically one that includes post and that may be deployable unilaterally. As shown, the devices and systems may include a fixation plate 10900; a coupling screw 10901, sized and configured to couple the fixation plate(s) to a fist lateral side of a spinous process; and a coupling backing 10902, sized and configured to couple to the opposite lateral side of the spinous process. As shown, the fixation plate may be positioned within the spine such that it is coupled to two adjacent spinous processes. The fixation plate may be positioned on a single lateral side of the spinous processes. The fixation plate may be attached to both spinous processes to stabilize the segment of the spine and promote fusion. The fixation plate may be coupled to the spinous processes by way of coupling screws for example. The screws may be fed through holes in the fixation plate and then into each of the spinous processes. As shown, the fixation plate further includes a post 10903. The post of this embodiment sized and configured to be positioned between two adjacent spinous processes. The post extends from the back side of the fixation plate (i.e. the side facing the spinous processes while implanted) to a lateral portion of the spine on the opposite lateral side of the spinous processes. For example, the post may extend from the back side of the fixation plate to about the facet joint on the opposite lateral side of the spinous processes. In some embodiments, the post may further function to distract the two spinous processes. The post may also be sized and configured to receive a graft material or may be made from a graft material. The post may function to deposit graft material across a portion of the opposite lateral side of the spine. The graft material may be made out of a bone graft material. The bone graft material may be machined or otherwise shaped. Alternatively, the bone graft material may be a mass of bone, such as chips or bone pieces, flexible cancellous, materials containing demineralized bone matrix (DBM), and/or materials containing bone morphogenetic proteins (BMP). Alternatively, the post may be filled with a filer material such as bone cement. As shown in FIG. 109, the bone graft may contact the spinous processes, the lamina, and/or the facet joint of the two adjacent vertebrae. In some instances, the graft will function to promote fusion of these spinal regions. In some embodiments, the spinous processes, the lamina, and/or the facet joint(s) may be decorticated prior to the implantation of the fixation plate and/or bone graft to also, in some instances, promote fusion.

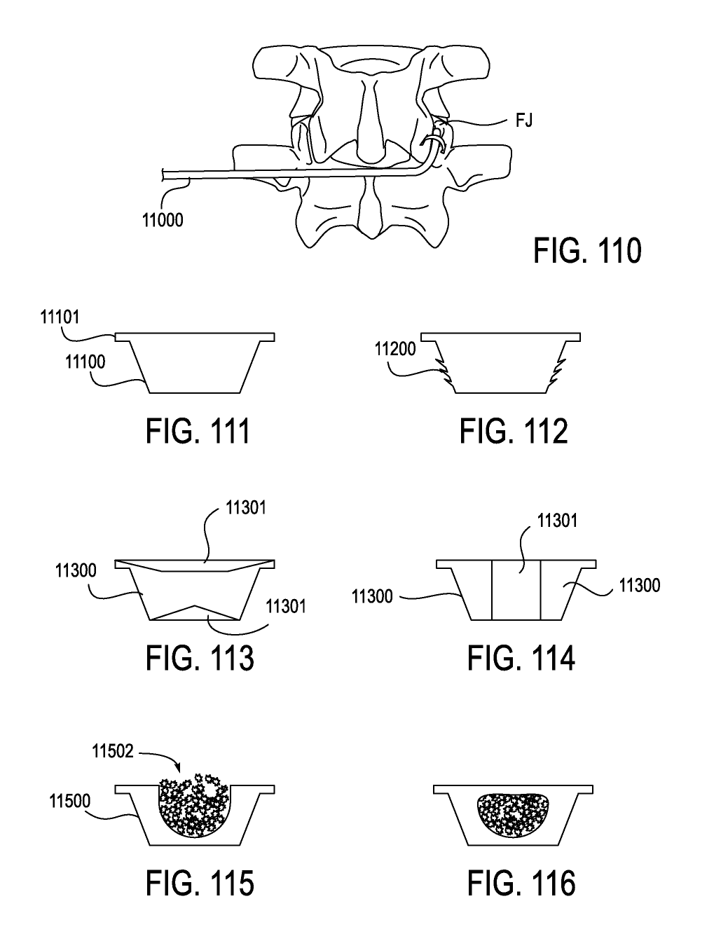

FIG. 110 shows an exemplary embodiment of a tool 11000 that may be used to prepare a facet joint (FJ) and that may be deployable unilaterally. As shown, the tool may be deployed from a first lateral side of the spinous processes, and may be used to modify tissue on the first lateral side of the spine and/or on the opposite lateral side spine (e.g. opposite side of the spinous processes). In some embodiments, the tool may be a drill or curette used to remove tissue (such as cortical bone) from the spine, such as from the facet joint. For example, the tool may deployed from a first lateral side of the spine, between two spinous processes, and may be used to roughen up the facet joint on the opposite lateral side of the spine. Additionally or alternatively, the tool may be used to inject material to the opposite lateral side of the spine, such as to the facet joint. For example, the injectable material may be a mass of bone, such as chips or bone pieces, flexible cancellous, materials containing demineralized bone matrix (DBM), bone cement, and/or materials containing bone morphogenetic proteins (BMP).

FIGS. 111-116 show exemplary embodiments of a graft implant. The implant may be made from a bone graft material or it may be sized and configured to receive a graft material. Alternatively, the bone graft material may be a mass of bone, such as chips or bone pieces, flexible cancellous, materials containing demineralized bone matrix (DBM), and/or materials containing bone morphogenetic proteins (BMP). Alternatively, the implant may be filled with a filer material such as bone cement. The bone graft material may be machined or otherwise shaped. For example, as shown in FIGS. 111 and 112, the graft material may include a tapered portion 11100 and a lip portion 11101. Alternatively, the implant may be sized and configured into any other suitable shape or configuration. In some embodiments, as shown in FIG. 112, the implant may include a coupling member 11200 such as teeth or prongs. In this example, the implant may be pushed down, while the prongs/teeth will prevent the implant from backing out (e.g. from moving up).

In some embodiments, as shown in FIG. 113, the tapered portion (or a portion thereof) may be made of a graft material 11300 capable of fusing with bone, while the lip material may be made from a material 11301 (such as metal (e.g. titanium) or plastic (e.g. PEEK)) that may not fuse with bone. Alternatively, the lip portion may fuse with bone and the tapered portion, or a portion of the tapered portion may not fuse with bone. As shown in FIGS. 113 and 114, different portions of the graft may be made out of a material 11301, while other portions may be made out of a material 11300. In some embodiments, the graft may comprise a third, fourth, or any other suitable number of materials. For example, as shown in FIGS. 113 and 114, material 11301 may be made from a material (such as metal (e.g. titanium) or plastic (e.g. PEEK)) that may not fuse with bone. Alternatively, material 11301 may be cortical bone that may fuse with bone less well than cancellous bone in some instances. As shown in FIG. 113, material 11301 that does not fuse, or fuses less well, will be positioned across the top and bottom portions of the graft, while in FIG. 114, material 11301 may be positioned through the center of the graft. In these configurations, material 11301 may provide increased stability and/or structure while it may prevent bony growth or fusion into areas, such as the spinal column, where fusion is not desired. FIGS. 113 and 114 are exemplary embodiments. Any other suitable combination of materials and configuration of materials may be acceptable such that they encourage and promote fusion in desired areas of the spine, provide structure and spinal stability, and/or prevent bony in-growth into non desirable areas.

FIGS. 115 and 116 illustrate exemplary embodiments of graft implants that may be sized and configured to receive a graft material. For example, the portion 11500 of the implant (labeled, for example, PEEK structure) may be machined or otherwise shaped. The outer structure may be made from a material (such as metal (e.g. titanium) or plastic (e.g. PEEK)) that may not fuse with bone. Alternatively, the outer portion of the graft may be cancellous bone or cortical bone, which may fuse with bone less well than cancellous bone in some instances. The bone material (particles 11501 in the figure) may be a mass of bone, such as chips or bone pieces, flexible cancellous, materials containing demineralized bone matrix (DBM), and/or materials containing bone morphogenetic proteins (BMP). Alternatively, the implant may be filled with a filer material such as bone cement. As shown in FIG. 115, the graft structure may define an open trough to be filled with bone material, and may optionally include a cap 11502 to cover the top portion of the trough. Alternatively, as shown in FIG. 116, the graft may have a hollow section through the width of the graft that is sized and configured to receive bone material. In some instances, there may be holes or windows along the sides of the graft (not shown) that allow the bone material and the bony portions of the spine to contact and potentially fuse together.

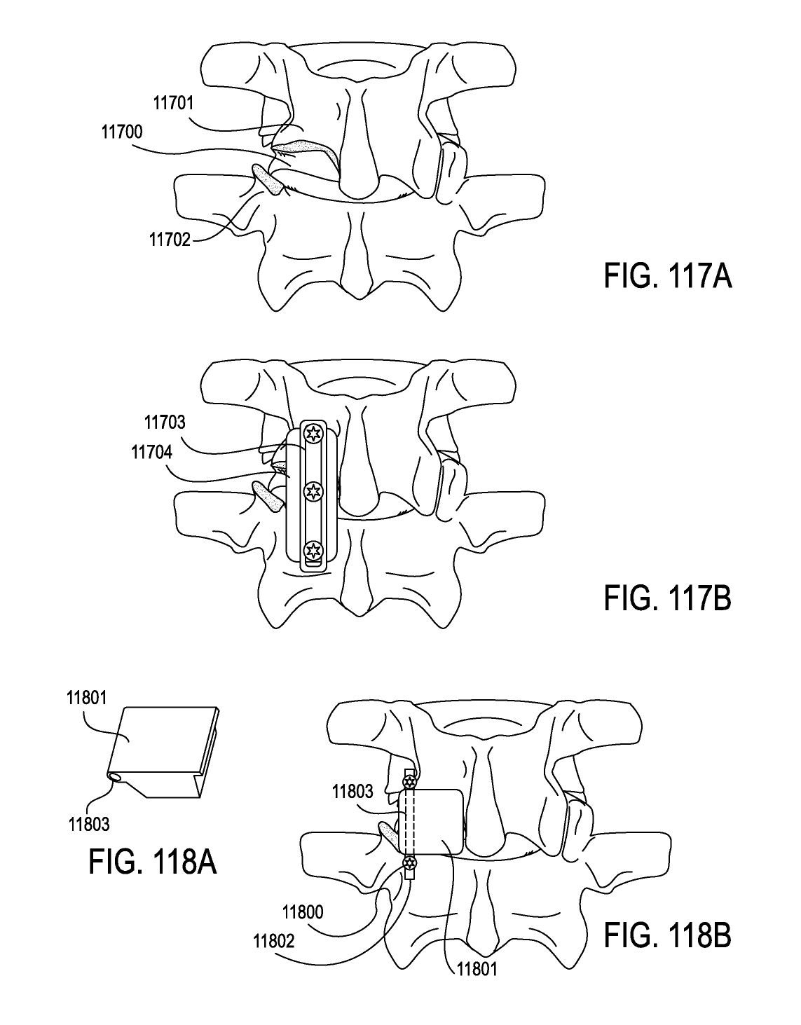

As shown in FIG. 117A, a Facetectomy (removal of a facet joint) 11700 may be performed on a patient's spine. Methods of performing a Facetectomy are described in detail in any of the patent applications and provisional patent applications listed above which are each incorporated by reference in their entirety. Specifically, exemplary methods of performing a Facetectomy are described in detail in U.S. patent application Ser. No. 12/773,595, entitled “TISSUE MODIFICATION DEVICES”, and filed May 4, 2010, which is hereby incorporated by reference in its entirety. As shown in FIG. 117B, the implant may be implanted coupling a portion of the pars of the lamina 11701 of a first vertebra to a superior articular process 11702 of a second, adjacent (caudal) vertebra. Alternatively, the implant may contact the spinous processes, the lamina, and/or the facet joint of the two adjacent vertebrae. In some instances, the graft will function to promote fusion of these spinal regions. In some embodiments, the spinous processes, the lamina, and/or the facet joint(s) may be decorticated prior to the implantation of the fixation plate and/or bone graft to also, in some instances, promote fusion.

In some embodiments, during compression of the spine (e.g. distraction is released and/or the spine (at least one level) is put into compression), the implant may be secured in place as it is wedged between two adjacent vertebrae. In some embodiments, the implant may be used without ipsilateral pedicle screws. Alternatively, as shown in FIG. 117B, a locking mechanism 11703 coupled to the implant 11704 may include screws implanted into the lamina of the adjacent vertebrae. The locking mechanism may function to hold or buttresses the implant in place in order to prevent the implant from expulsion or impinging on the spinal cord. Alternatively, a pedicle screw (11800 in FIG. 118B) may be driven through the implant and into the pedicle on the ipsilateral side, for example. In an alternative embodiment, the implant may be coupled to a fixation plate of an embodiment of interspinous fusion device. For example, a bracket of an interspinous fusion device (see for example, FIG. 7 of U.S. Provisional Patent application No. 61/357,529, entitled “SYSTEMS AND METHODS FOR PERFORMING SPINAL FUSION”, and filed Jun. 23, 2010, which is hereby incorporated by reference in its entirety) may be coupled to the implant (and/or an adjacent pedicle) via a fixation screw.

In some embodiments, the implant may provide at least the following benefits. For example, the implant may provide scaffolding that may enable fusion, such as interlaminar fusion for example. Additionally, the implant may function to replace a facet joint. The implant may also prevent the need for ipsilateral (same sided) pedicle screws. The implant may provide an advantage in either the prevention or minimization of lateral bending and/or axial rotation. Furthermore, the implant may function to maintain an expanded spinal canal, secure spinal stability, preserve the protective function of the spine after a laminoplasty (or Facetectomy) has been performed, and/or any combination thereof.

FIGS. 118A and 118B show an embodiment of a graft implant 11801 configured to receive a connecting rod 11802. As shown, a locking mechanism in this embodiment includes at least one pedicle screw 11800 and a connecting rod 11802. As shown in FIG. 118A, the graft may be configured to receive the connecting rod. For example, the graft may include a hole 11803 along the length of the graft. As shown in FIG. 118B, the pedicle screws may be implanted into the spine, and then a rod (fed through the graft) may be connected to the pedicle screws.

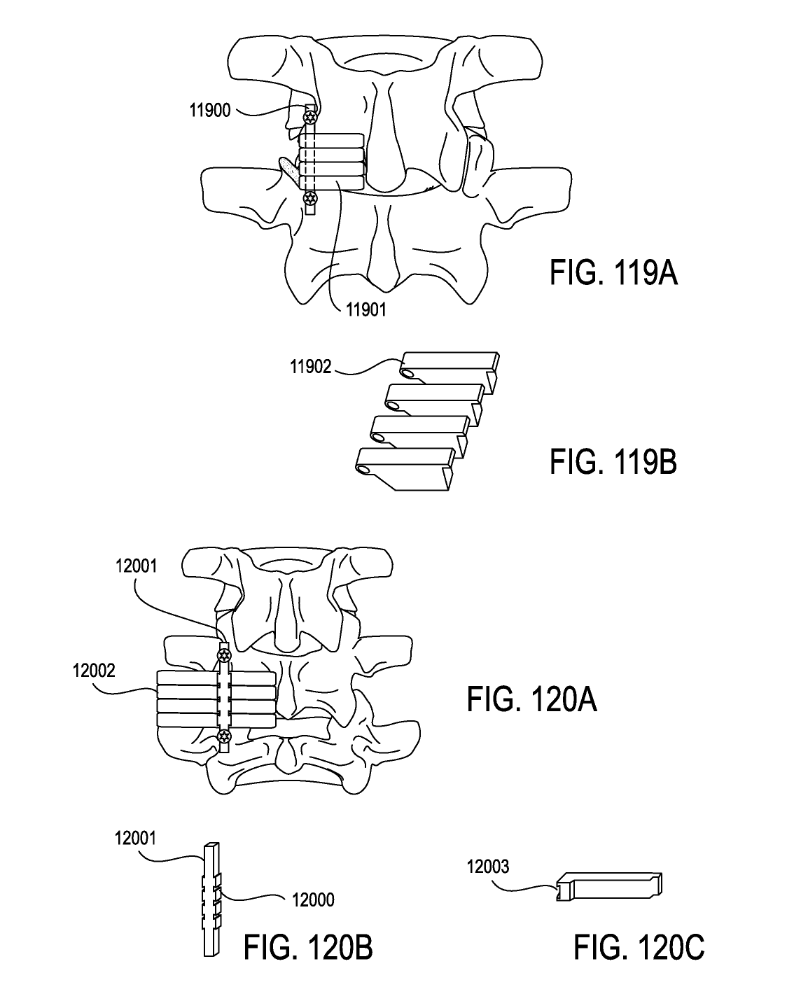

FIGS. 119A and 119B show an embodiment of a modular graft implant 11901 configured to receive a rod 11900. This device and method may be similar to the devices described above in reference to FIGS. 118A and 118B, however the graft may be comprised of multiple modular sections that can be coupled together to form a graft implant. In use, a surgeon may select to couple any number of graft sections together depending on the anatomy or clinical need of the patient. In some embodiments, the graft implant may be comprised of multiple graft sections of different sizes. As described above and as shown in FIG. 119B, the graft sections may each be configured to receive the connecting rod. For example, the graft may include a hole 11902 along the length of the graft. The graft sections may be coupled to one another before, during, or after the rod is coupled to the graft. Alternatively, the graft sections may not be coupled together. In some embodiments, rods of different lengths may also be provided.

FIGS. 120A to 120D show an embodiment of a modular graft implant configured to couple to a rod. This device and method may be similar to the devices described above in reference to FIGS. 119A and 119B; however the multiple modular sections may be coupled to the rod with an alternative mechanism. For example, as shown, the graft sections 12002 may each be configured to receive a coupling portion 12000 of the connecting rod 12001. For example, the graft may include a slot 12003 across the height of the graft. The graft sections may be coupled to one another before, during, or after being coupled to the rod. Alternatively, the graft sections may not be coupled directly together. In some embodiments, rods of different lengths may also be provided. Although not shown, any of the modular graft sections described herein may be configured as the graft implants as described in FIGS. 111-116, or any other suitable graft configuration.

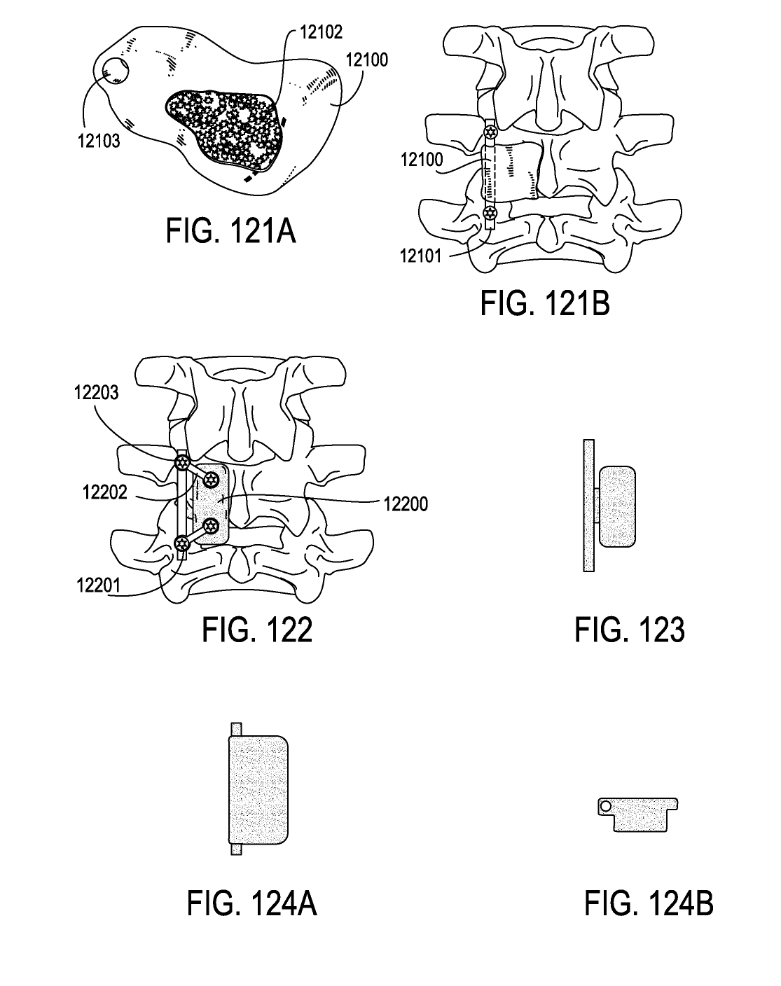

FIGS. 121A and 121B show an embodiment of a flexible graft implant 12100 configured to receive a rod 12101. In this embodiment, the graft implant may comprise a flexible outer structure that is sized and configured to receive bone material (e.g. through hole 12103). The bone material (particles 12102 in the figure) may be a mass of bone, such as chips or bone pieces, flexible cancellous, materials containing demineralized bone matrix (DBM), and/or materials containing bone morphogenetic proteins (BMP). Alternatively, the implant may be filled with a filer material such as bone cement. The outer structure may be a flexible bag, sack, pouch, etc. that is configured to conform to spinal anatomy and fill a desired space within or around a spine. The sack may be a porous or permeable material such that the bone material within the sack may fuse with bone outside of the sack. The sack may be configured such that only portions of the sack are permeable. The sack may be deployed minimally invasively, e.g. through a small tube, and then may be deployed to its full size once it's in place. In some embodiments, before, during, or after deployment of the sack, the sack may be filled with bone material and coupled to a connecting rod, as shown in FIG. 121B.

FIG. 122 shows an embodiment of a graft implant 12200, a rod 12201, and a locking mechanism 12202. This device and method may be similar to the devices described above in reference to FIGS. 117B or 118B with an alternative locking mechanism. In this embodiment, a locking mechanism may include at least one pedicle screw 12203 and a connecting rod 12202. As shown in FIG. 122, the pedicle screws may be implanted into the spine, and then a rod may be connected to the pedicle screws. As shown in FIG. 122, the locking mechanism includes a bar or rod that couples the pedicle screw to a screw coupled to the graft. This connection between the rod and the graft may be adjustable and for example, may rotate in roll, pitch, or yaw axes.

FIGS. 123 to 124B show exemplary embodiments of a graft implant with an integrated rod. In this embodiment, as shown in FIG. 123, the graft implant is machined or shaped to include a rod like structure that may be coupled to pedicle screws, or other suitable locking mechanisms. In FIG. 124A the graft implant is also machined or shaped to include a rod like structure. In this variation, the rod portion extending from the graft body may have a circular cross section, as shown in FIG. 124B.

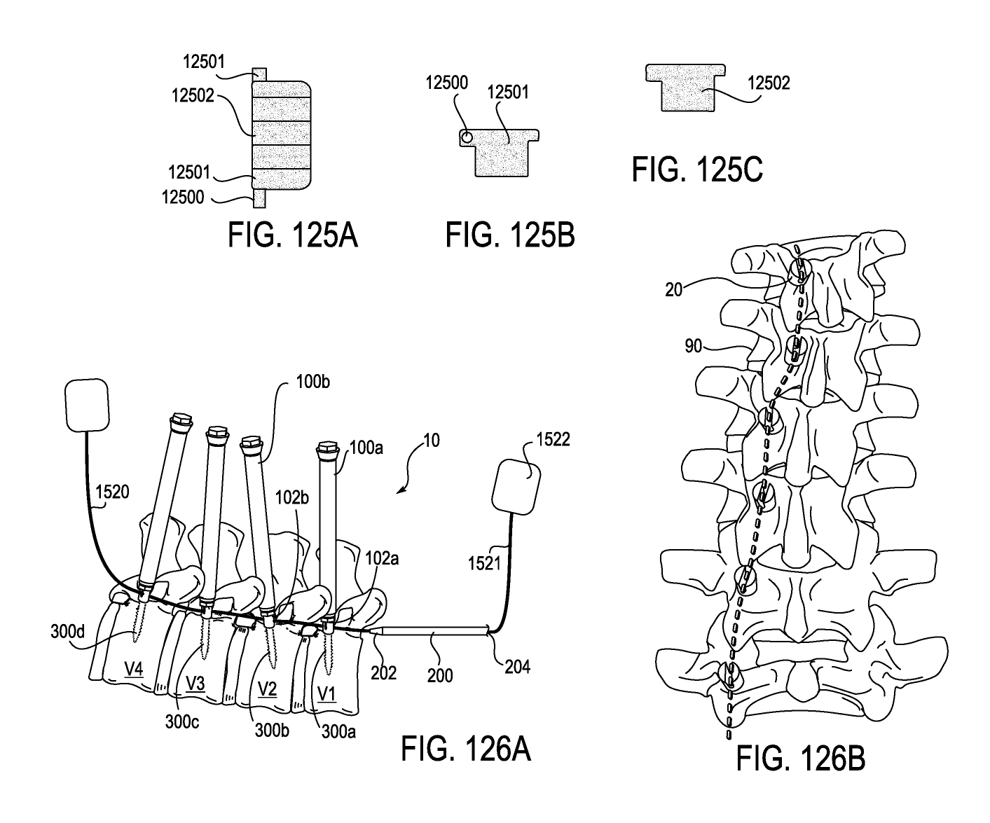

FIGS. 125A to 125C shows an exemplary embodiment of a modular graft implant with an integrated rod 12500. This device and method may be similar to the devices described above in reference to FIGS. 124A and 124B, however the graft may be comprised of multiple modular sections that can be coupled together to form a graft implant. In this embodiment, the graft may include at least two distinct graft sections. For example, as shown in FIG. 125B, the outer graft section 12501 (positioned toward the most caudal and cephalad positioned) may include a rod portion as described above in reference to FIG. 124B, for example. In this variation, the rod portion extending from the outer graft section may have a circular cross section. The center graft sections 12502, as shown in FIG. 125C, may be coupled to the outer graft sections and may not include rod portion extensions.

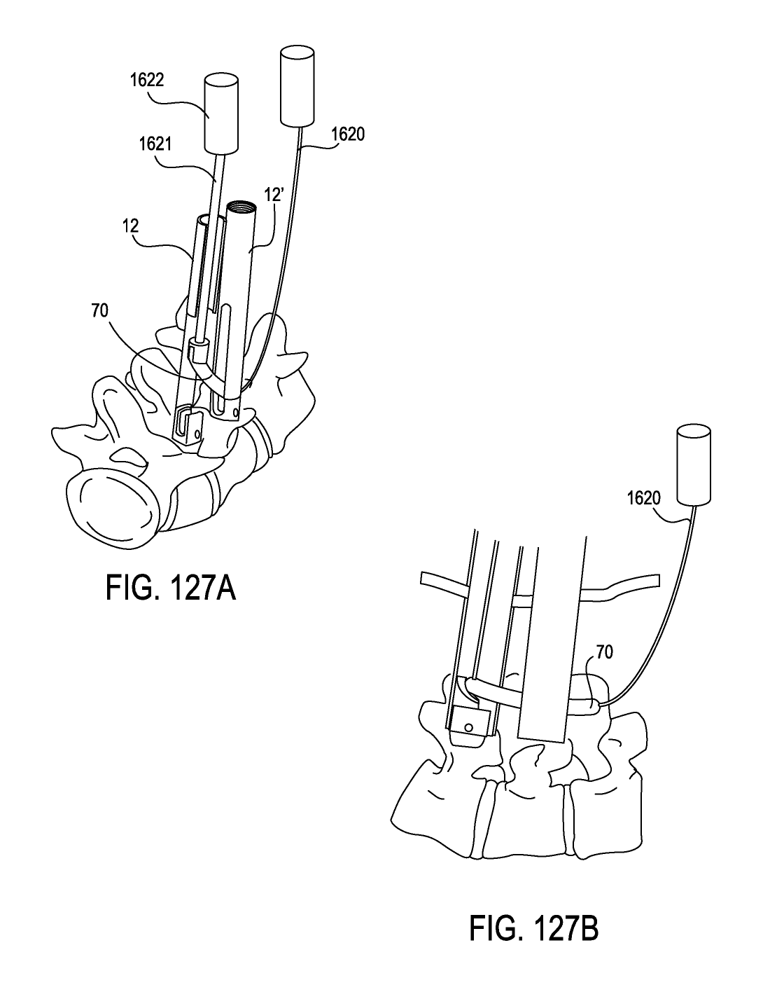

Also described herein are minimally invasive methods and devices for introducing spinal fixation elements into a patient's spinal column. Spinal fixation elements may include rods (or other suitable connectors), screws, anchors, or other suitable spinal stabilization devices, as shown in FIGS. 126A-127B. For example, a system for inserting theses devices may include a probe for inserting and positioning a guidewire/pullwire, a pullwire that is adapted to couple to the distal end of a connector (or a carrier for the connector), a connector (or rod or other spinal stabilization element), and a connector delivery tool that also holds the connector and may include a proximal handle or manipulator. Examples of a probe is shown in FIG. 94E.

FIG. 126A illustrates a minimally invasive surgical system 10 that includes a pullwire 1520 that is adapted to couple to the distal end of a connector 200, a connector delivery tool 1521 that also holds the connector 200 and may include a proximal handle 1522. The connector may be used in a system, such as one described, for example, in US Patent Application 2009/0182382. This system may include four (or any suitable number) of anchors 300a, 300b, 300c, 300d that are engaged to respective ones of the vertebrae V1, V2, V3, V4. In some variations, the system may include extensions 100 that can include a length extending proximally from the respective anchors 300 so that at least the proximal ends thereof are located outside a respective wound or incision in the patient through which a respective one of the anchors 300 is positioned to engage the respective vertebra.

The pullwire 1520 is movable along a percutaneous insertion path that starts at a location remote from the extensions 100 through skin and tissue of the patient to position connecting element 200 in a location adjacent the anchors 300. In this system, connecting element 200 can be an elongated brace, rod or shaft that is generally linear along its length to facilitate placement between two or more anchors. Alternatively, the connecting element 200 can be curved along all or a portion of its length. Connecting element 200 (that is insertable using pullwire 1520) may include any configuration known for a rod or implant. Further, connecting element 200 can be non-rigid, elastic and/or super-elastic and in the form of a cable, band, wire, or artificial ligament that is used in tethering, guiding, or other surgical procedures. Connecting element 200 can be percutaneously or non-percutaneously inserted with pullwire 1520 to a location adjacent connecting element engaging portions of one or more anchors engaged to a bony structure in the body of an animal subject to stabilize the bony structure.

In FIG. 126A the leading end 202 of connecting element 200 is shown in an approach to anchor 300a with trailing end 204 of connecting element 200 engaged to connector delivery tool 1521. Extension 100a forms a space 102a adjacent anchor 300a for receiving connecting element 200. By pulling on pullwire 1520, the surgeon can manipulate leading end 202 and connecting element 200 through the tissue of the patient and through space 102a toward space 102b formed between extension 100b and anchor 300b for placement therethrough. When connecting element 200 is positioned between the desired number of anchors, connecting element 200 can be engaged to the anchors with a suitable engaging member, such as a set screw, nut or other engaging member. In a further embodiment, connecting element 200 is seated relative to the anchors by operation of one or more of the extensions 100 before engaging connecting element 200 to anchors 300. Such seating can take the form of a spinal reduction procedure where one or more vertebrae are pulled or moved into alignment, and then engaged and maintained in this alignment by engagement of connecting element 200 thereto via the anchors 300.

As shown in FIG. 126B, the system is used for correcting a spinal deformity as described, for example in WO/2009/046046. In this variation, the devices may include a connector attached to one or more vertebral members of a deformed spine. As shown, anchors 20 are initially attached to the vertebral members 90, such as within the pedicles. The connector may be constructed of a flexible material with elastic properties. The connector may be attached to the vertebral members, as shown, in a stressed orientation. Due to the elastic properties of the material, the member exerts a corrective force on the vertebral members. In some embodiments, multiple members are attached to the vertebral members to apply the corrective force. A pullwire may be coupled to the elastic connector and pull the connector along a path A to position the connector through the anchors.

FIGS. 127A and 127B illustrate a minimally invasive surgical system that includes a pullwire 1620 that is adapted to couple to the distal end of a connector 70, a connector delivery tool 1621 that also holds the connector 70 and may include a proximal handle 1622. The connector may be used in a system, such as one described, for example, in US Patent Application 2009/0138056. As shown in FIGS. 127A and 127B, in general, the method involves advancing a spinal fixation element in a lengthwise orientation along a minimally invasive pathway that extends from a minimally invasive percutaneous incision to a spinal anchor site. As described above, the spinal fixation element is advanced by pulling on a pullwire 1620 (as shown in FIG. 127B). In an exemplary embodiment, a percutaneous access device 12 is used to create the minimally invasive pathway for receiving the pullwire and the spinal fixation element and for delivering the fixation element to a spinal anchor site. The spinal fixation element is preferably inserted through a lumen in the percutaneous access device in a lengthwise orientation, such that the spinal fixation element is oriented substantially parallel to a longitudinal axis of the percutaneous access device. As the spinal fixation element approaches or reaches the distal end of the pathway, the pullwire 1620 pulls the spinal fixation element to orient it at a desired angle with respect to the percutaneous access device 12, preferably such that the spinal fixation element is substantially parallel to the patient's spinal column (as shown in FIG. 127B). The spinal fixation element can then optionally be positioned to couple it, either directly or indirectly, to one or more spinal anchors. A fastening element or other closure mechanism, if necessary, can then be introduced (for example, via the pullwire) into the spinal anchor site to fixedly mate the spinal fixation element to the anchor(s).

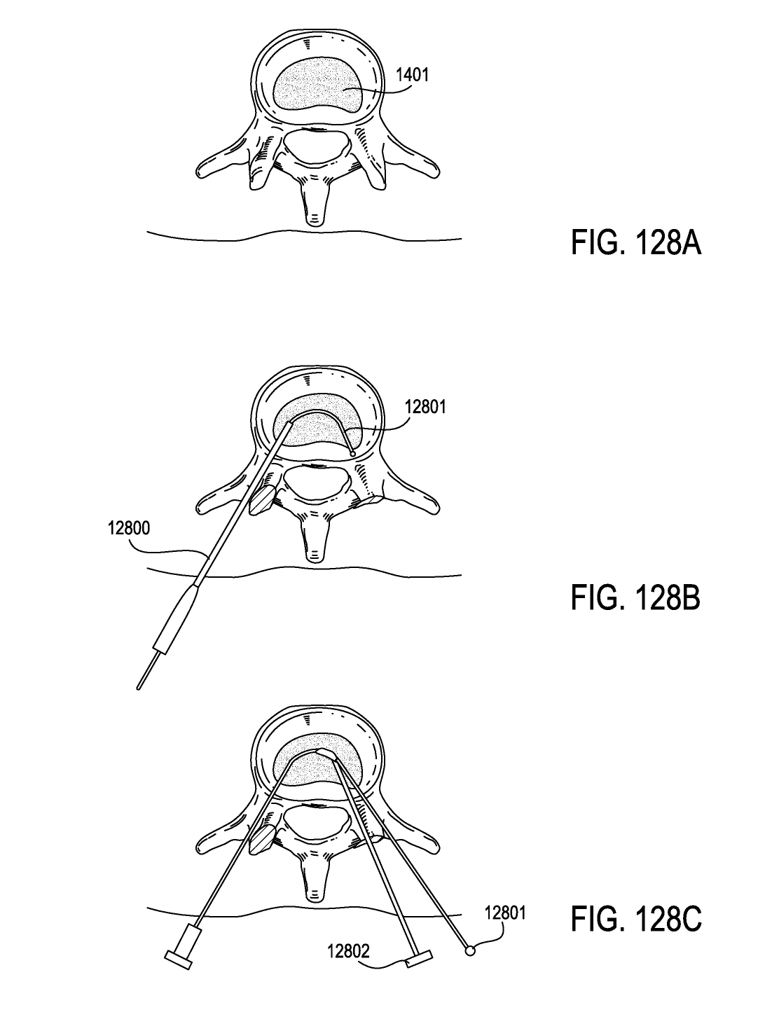

The systems and methods described herein may also be used as part of a Posterior Lumbar Interbody Fusion (PLIF) procedure. Unilateral posterior or posteriorlateral approaches to access the disc space can be less invasive than bilateral approaches but instrument and implant positioning can be challenging. The following methods may be utilized in any fusion procedure from any suitable approach or combination of approaches. For example, it may be difficult to compete a discectomy contralaterally and position a single TLIF cage or posterior disc replacement across the appropriate disc space, as illustrated in FIG. 128A. The endplates are heterogeneous, and thus misplaced implants may not have the best contact with dense cortical bone 1401, placing them at greater risk for subsidence. To address this issue, a bimanually controlled pullwire system can be used to guide and pull instruments and implants into proper position in the disc space.

For example, FIGS. 128B and 128C illustrate one variation of a PLIF-type procedure that is made more effective using the pullwire techniques described herein. In FIG. 128B, for example, the guidewire/pullwire is first positioned in the disc space using a probe or probes 12800, as described above. In one variation, a cannulated probe having a curved distal end is inserted contralaterally and percutaneously in to the disc space, toward an ispilateral direction. The pullwire 12801 may then be passed through the probe and out of the disc on the ipsilateral side. As illustrated in FIG. 128B, the procedure may be combined with a TLIF procedure in which part of the ipsilateral facet joint has been removed.

The pullwire may then be placed through the probe and extended distally out of the disc. In FIG. 128B, the pullwire extends distally from the ipsilateral incision (the excised region). Once the pullwire is in position, it may be used to pull one or more instruments or device (e.g., implants, cages, fillable/expandable structures, etc.) into place, as described above. In some variations a spacer or distractor 12802 may be used to open the disc space, as illustrated in FIG. 128C.

In any of the variations described herein, the method may also include the insertion of a pivot that may help guide the pullwire and/or devices pulled by the pullwire. For example, in FIG. 128C the distractor element may act as a pivot point to help control the ventral/dorsal location of the pullwire as it is manipulated. In this variation, the distractor is a pivot that is configured as a “rapid exchange” elongate element; the distal end of the pivot is configured to couple with the pullwire so that it can be pushed along the pullwire, yet still allow the pullwire to be pulled distally and proximally through or around the pivot. In some variations the pivot includes a distal channel for the pullwire. As illustrated in FIG. 128C, the pivot may be inserted from either the proximal or distal end of the pullwire (typically after it has been initially positioned using the probe) and slide along the pullwire until it is positioned at the desired pivot point. Once in position, it may be held in place (e.g., anchored) from within the body or from outside of the body (e.g., by a clamp or other anchor), or simply held in place. In the variation shown in FIG. 128C, the pivot is also a distractor, and thus may be used to separate tissues (e.g., bone). In other variations the pivot is anchored or anchorable in the body, and provides a surface against which the pullwire may move without allowing substantial migration of the pullwire from the pathway through the body.

As mentioned, described herein are devices, systems and method for treating tissue by first placing a guidewire (or “pullwire”) in position within the body, and then using the guidewire to position, anchor and/or treat the tissue. In general, these methods and systems are “bimanual” procedures, in which the implant or tissue modification device is controlled within the body from one or more separate locations outside of the body, and by manipulating the implant/device from both the distal and proximal ends. These systems and methods may be particularly useful for percutaneous or tube based treatments of one or more body regions. However, it should be understood that any of the devices, methods and systems described herein may be used as part of an “open” or “mini-open” surgical procedure in which access to a body region is created through an opening in the tissue (e.g., by removal of tissue). Any of the systems and devices described may be performed as part of a procedure that is at least partially open. Partially percutaneous procedures may also be performed using these devices, systems and methods.

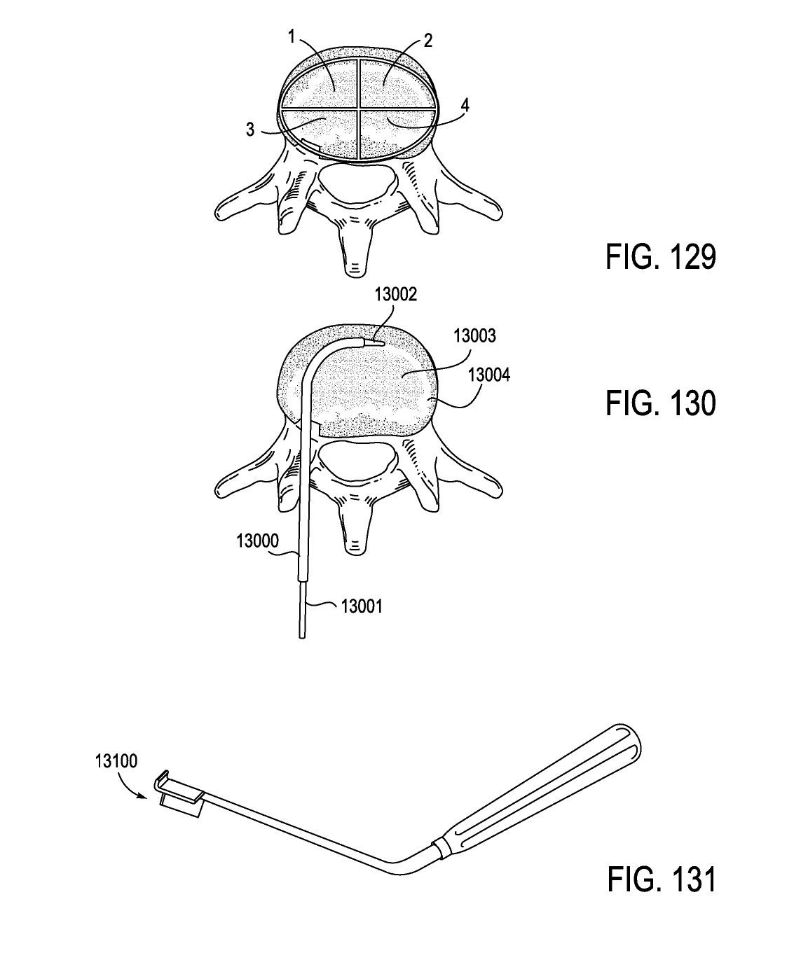

As mentioned, the systems and methods described herein may also be used as part of a TLIF or XLIF, for example. Specifically in a TLIF, it may be difficult to complete a discectomy contralaterally (on the opposite side from the access location) and position a single cage or posterior disc replacement across the appropriate disc space. The endplates are heterogeneous, and thus misplaced implants may not have the best contact with dense cortical bone, placing them at greater risk for subsidence. It may be preferable to position the implant such that it is positioned within at least one posterior quadrant of the disc and one anterior quadrant of the disc. As shown below in FIG. 129, a disc has four quadrants—two anterior 1, 2; and two posterior 3, 4. In may be even more preferable, in some embodiments, to position the implant such that it is positioned within all four quadrants. To address this issue, a bimanually controlled pullwire system can be used to guide and pull instruments and implants into proper position in the disc space.

Furthermore, is may be difficult to remove a sufficient amount of disc material, especially contralateraly from the access point. In general, it is understood that the more disc material removed, the higher the likelihood of a successfully placed implant and of a successful bony fusion occurring. Therefore, it is important that a sufficient amount of disc material be removed. The system described herein allows for improved and/or complete removal of the desired tissue, particularly from the typically hard to reach contralateral side.

Additionally, it is typically not easy or even possible to remove an implant, once it has been deployed. The system described herein allows for removal and redeployment of implants.

FIGS. 130-155 illustrate exemplary methods, devices, and systems for removing tissue and delivering an implant. In particular, the methods, devices, and systems illustrated are for removing spinal disc tissue (e.g. performing a discectomy) and delivering an interbody implant typically as part of a Spinal Fusion Procedure and/or Disc Replacement Procedure.

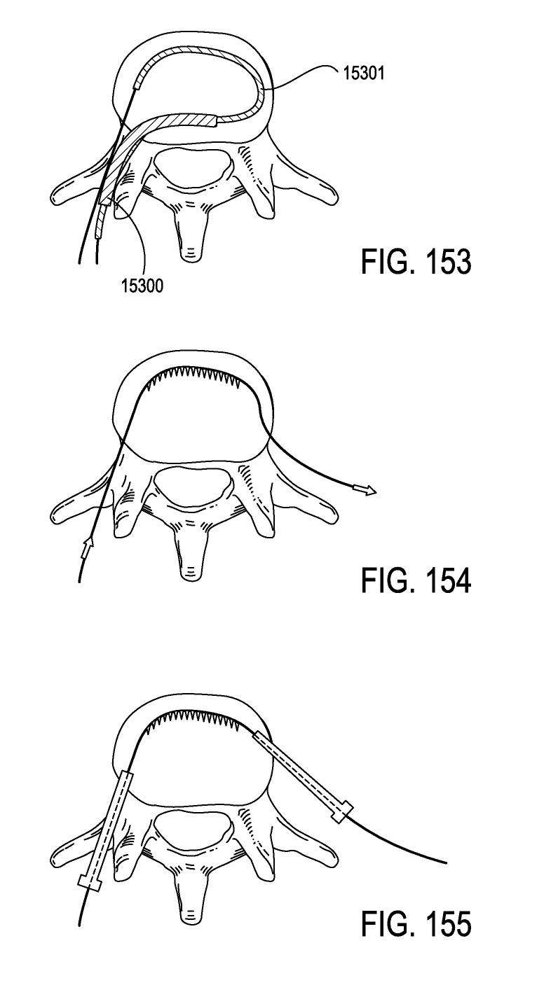

As shown in FIG. 130, a method for removing tissue may begin with the step of inserting an access device, such as a probe 13000 having an inner cannula 13001 and a probe tip 13002, into an intervertebral disc of a spine of a patient. Alternatively, as shown in FIG. 153, the probe 15300 may initially positioned toward the posterior aspect of the disc. The spine may be accessed through a single access point. For example, a midline or paramedian skin incision may be made. A tube, such as a METRx tube (Medtronic, Inc.), and/or muscle retractors may be used to provide access to the spine through the incision. Alternatively, the probe may be percutaneously advanced to the spine and into the disc and/or may be advanced into location without any additional instruments. In some embodiments, the probe (and device as described below) may be advanced with or behind a shield, as shown in FIG. 131. The shield 13100 may function to protect nerves and other non-target tissue from damage while the devices and implants are inserted and/or while material is removed from the disc.

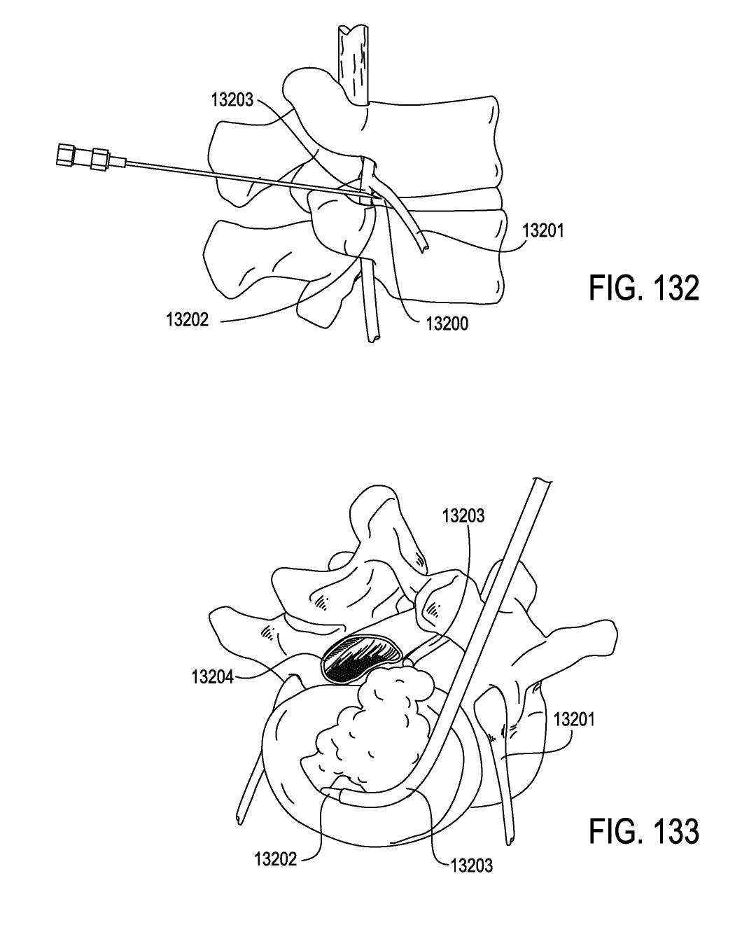

A working space may be exposed, such as what is known in the art as Kambin's Triangle 13200, at the inferomedial aspect of the spinal neural foramen. As shown in FIGS. 132 and 133, Kambin's triangle is generally defined as a right triangle over the dorsolateral disc. The hypotenuse is the exiting nerve 13201, the base (width) is the superior border of the caudal vertebra 13202 (e.g. the superior margin of the caudal pedicle), and the height is the traversing nerve root 13203 (or dural sac 13204). The method, and specifically the access step, may be carried out while using image guidance and/or tracking (e.g., electromagnetic tracking). To advance the probe to the disc, a portion of the facet joint, lamina, ligament (e.g. longitudinal ligament or ligamentum flavum), etc. may be removed. Alternatively, the probe may be angled such that it may be inserted without removing any additional tissue.

As shown in FIG. 130, an intervertebral disc may include a nucleus pulposus 13003 surrounded by an annulus fibrosus 13004. A portion of the annulus may be removed prior to the insertion of the probe, or alternatively, the probe may be inserted directly through the annulus. The probe may be inserted such that within the disc the probe, or a portion thereof, curves around the nucleus. The probe may be positioned within the annulus, or just inside of the annulus within the nucleus. The probe tip may be configured such that it can be advanced through the annulus and/or the nucleus. For example, the probe tip may be sharp or may alternatively be configured for blunt dissection or penetration through the disc.

In general, the distal end of the outer cannula of the probe described herein may be curved or bent, and/or may be curveable or bendable. The inner movable catheter may be configured to bend as it exits the distal end of the outer cannula, as shown, thereby increasing the ability of the probe to guide a guidewire around a target tissue (e.g. disc nucleus). The movable catheter may include a nitinol component such that, as the movable catheter is advanced out of the outer cannula (FIG. 136), it assumes a curved shape and continues around the disc and around the target tissue.

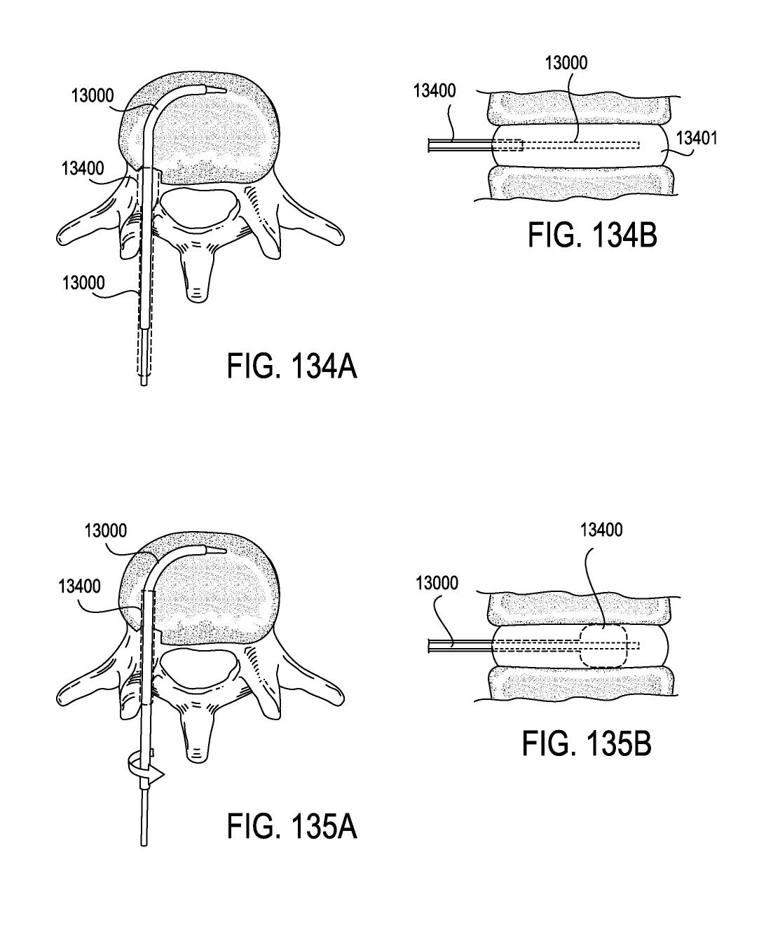

Once the probe is positioned within the disc, a distracting and/or locking mechanism 13400 may be positioned within the disc. The locking mechanism may function to lock the outer cannula in position with respect to the target tissue. Although the outer cannula may be locked into position, the inner movable catheter may be movable within the outer cannula, as shown in FIG. 136. The distracting mechanism may function to distract a first vertebra and vertebral end plate from a second, adjacent vertebra and vertebral end plate. As shown in FIG. 134B, an intervertebral disc 13401 is positioned between a first superior vertebral body and a second inferior vertebral body. As shown, an intervertebral disc includes an outer layer of articular cartilage that interacts with the end plates of the vertebral bodies. In some embodiments, as shown, a single device or device component may function to both lock and distract. In some embodiments, distraction may be achieved by an external retractor, laminar spreader, or by implanting pedicle screws in such a way to distract the vertebral bodies from one another.

In some embodiments, a distracting and/or locking mechanism may be advanced over the outer cannula of the probe, as shown in FIGS. 134A and 134B. Alternatively, the outer cannula may include an integral distracting and/or locking mechanism. For example, the locking/distracting mechanism 13400 may include a balloon or other suitable expandable element such as a mesh or braid. As shown in FIGS. 134A and 134B, an over tube is advanced over the outer cannula of the probe. As shown in the axial view of FIG. 134A the over tube includes a distal portion having a diameter or width wider than the diameter of the proximal portion of the over tube. For example, the distal portion may be a disk having a circular or elliptical shape. As shown in the anterior/posterior view of FIG. 134B, the distal portion may be disk shaped such that is has a thickness or height substantially equal to the height of the proximal portion of the over tube. Alternatively, the distal portion may be expandable, such that in its expanded shape, it has a diameter larger than the proximal portion of the over tube. The largest (or expanded) width of the distal portion is preferably larger than the equilibrium distance between the first superior vertebral body and a second inferior vertebral body, such that the distal portion of the over tube may function to distract the vertebral bodies, thereby providing a working space for the removal of tissue and the insertion of an implant.

As shown in FIGS. 135A and 135B, the over tube 13400 (or cannula in an alternative variation) may be rotated with respect to the intervertebral disc such that the wider diameter is positioned between the two vertebral bodies, thereby distracting them from one another. Alternatively, a portion of the over tube (or cannula) may be expanded such that the expanded width is larger than the equilibrium distance between the first superior vertebral body and a second inferior vertebral body, such that the vertebral bodies are distracted from one another.

Once the outer cannula of the probe is locked in place with respect to the disc and/or the vertebral bodies have been distracted from one another, the inner catheter 13001 may be advanced within the outer cannula 13000 and deployed within the disc, as shown in FIG. 136. Alternatively, the inner cannula may be advanced at any other suitable time within a procedure. As described above, the movable catheter may include a nitinol component such that as the movable catheter is advanced out of the outer cannula, is assumes a curved shape and continues around the disc and around the target tissue. The inner catheter may also be curved along the inside of the annulus by the rigidity of the disc annulus. The catheter may be advanced such that the probe tip is oriented back toward the access point and/or a more proximal portion of the outer cannula of the probe. Alternatively, as shown in FIG. 153, where the outer cannula 15300 of the probe is initially positioned toward the posterior aspect of the disc, the inner catheter 15301 may be advanced such that it curves toward the anterior portion of the disc, and in some embodiments, curves back toward the posterior access location.

In some embodiments, it may be desirable to lock the deployed catheter with respect to the disc. For example, the catheter may include an expandable locking mechanism as described above. Alternatively, a second over tube may be advanced over the first over tube, outer cannula, and catheter such that once in position toward a distal portion of the catheter, it may be rotated, as described above, to lock the catheter in place. Alternatively, the catheter may include an anchor, such as a hook or barb, deployable into the disc annulus, to hold the catheter in place with respect to the disc. In some embodiments, the outer cannula may include an anchor rather than a rotatable or expandable locking mechanism as described above.

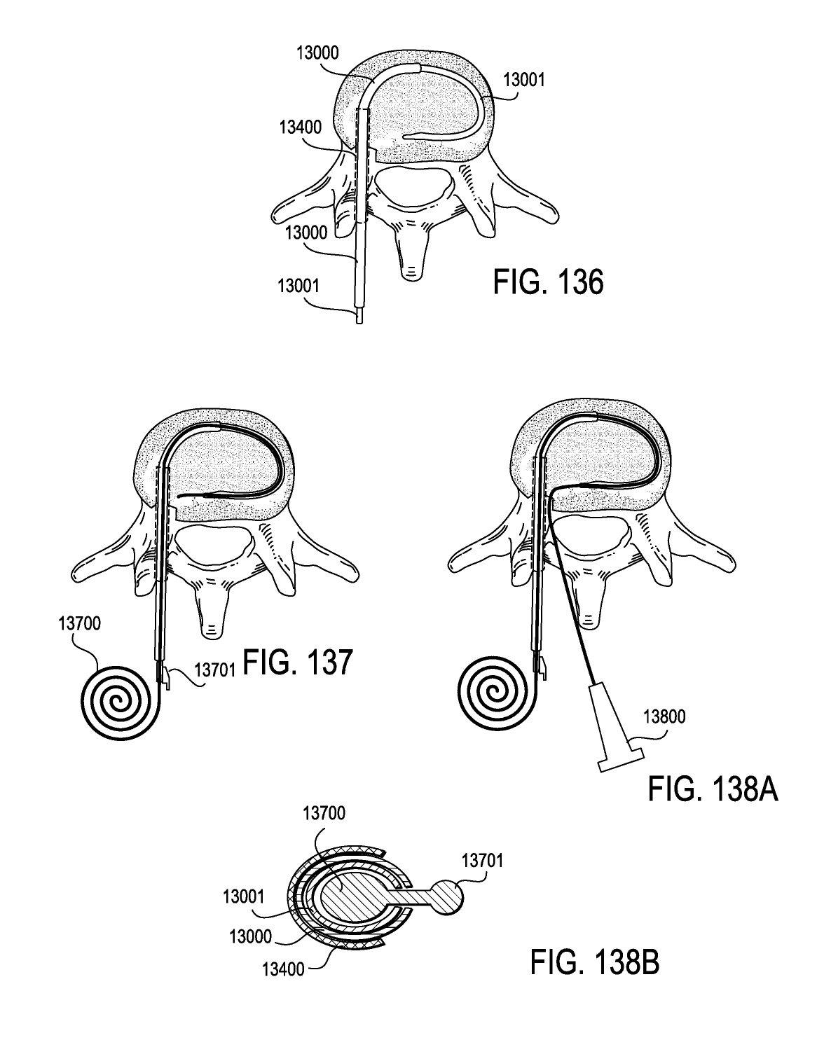

Once the probe is deployed and locked in position with respect to the disc, a wire 13700 may be advanced into the disc through the probe, as shown in FIG. 137. The guidewire may be advanced along the curved path of the probe such that it is placed around the target tissue of the disc. The guidewire may be advanced within the probe until it exits through the probe tip at the distal end of the catheter. In some embodiments, once the guidewire tip can be visualized (via direct visualization or fluoroscopy, for example) through the access point/opening through the disc annulus, the distal end of the guidewire may be grasped by a surgical instrument and pulled such that it exits the disc and the patient. Alternatively, the outer cannula and/or over tube may be configured to guide the guidewire from the disc. For example, the cannula or over tube may include a track or indentation that may function to bend the distal tip of the guidewire toward the access point/opening through the disc annulus, such that as the guidewire is advanced, it may turn toward the access point and continue out thought the access point. As shown in FIG. 137, the guidewire may exit through the same posterior access location through which the probe and other devices were inserted. Alternatively, as shown 24, the probe and/or guidewire may be positioned such that the guidewire exits contralateral from the access location.

The guidewire, as shown in FIG. 137, is typically long (e.g., elongated) and flexible, and may have a sharp (tissue penetrating) distal end and a proximal end that allows it to be coupled to a device, such as a tissue modification device, securely. For example, in FIG. 137, the guidewire includes a hitch 13701 or other shaped end for coupling to a device, such that the device can be pulled along by the guidewire. As described, the distal end of the guidewire may be completely withdrawn from the patient, so that it can be grasped and manipulated. As shown in FIG. 138A, a handle 13800 may be attached to the distal end of the guidewire. Similarly, the proximal end of the guidewire may be configured to pass through the probe and be external to the patient, so that it can also be grasped and manipulated.

As shown in the cross sectional view of FIG. 138B; the probe catheter 13001, the probe cannula 13000, and the over tube 13400 may all include a slit or opening disposed along their length. The slit may function to allow the guidewire 13700 to be advanced within the probe and over tube while the hitch 13701 of the guidewire (and therefore a device coupled to the hitch) may run along the outside of the probe and over tube while the hitch is coupled to the guidewire through the slit, as shown in FIG. 138B.

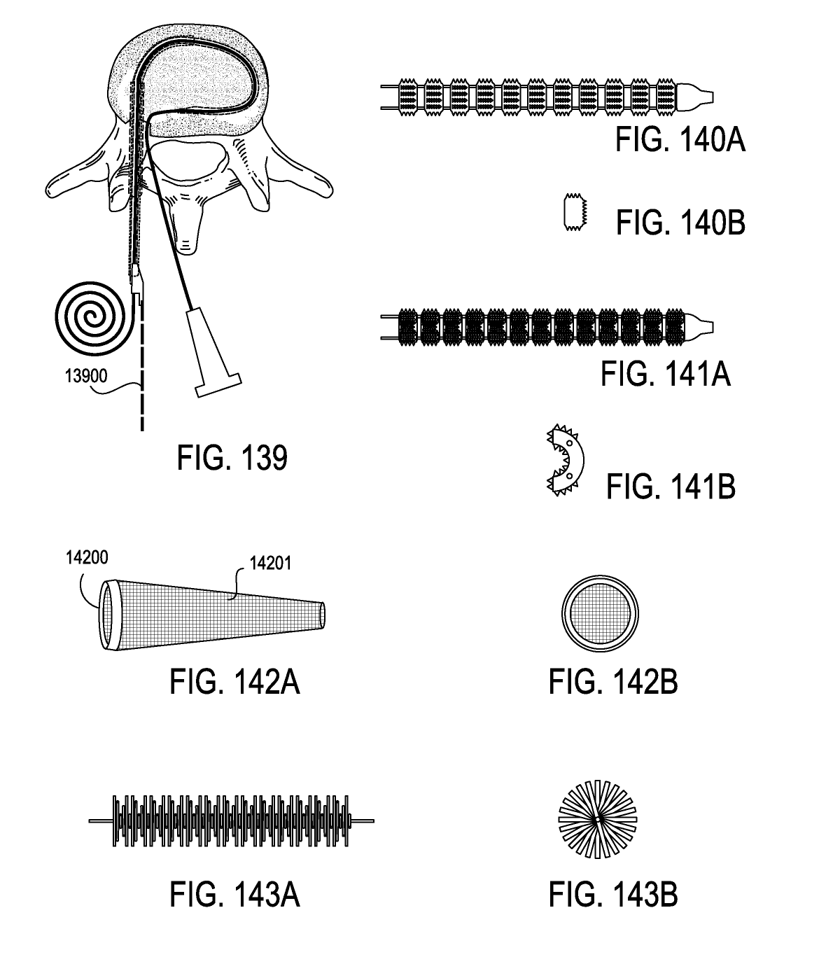

As shown in FIG. 139, a tissue modification device 13900 may be coupled to the hitch of the guidewire. As shown, the tissue modification device may be advanced into the disc by pulling on the distal handle coupled to the distal end of the guidewire. Alternatively, the tissue modification device may be pushed into place from its proximal end while pulling on the distal end of the device (via the guidewire, for example).

Various embodiments of tissue modification devices and systems are provided herein. In general, a flexible tissue-modification device as described herein is configured to remove tissue from a patient. In particular, these tissue-modification devices may be configured to modify and/or remove the nucleus and/or annulus of the disc and/or prepare a vertebral end plate (e.g. roughening and/or removal).

These devices typically include a flexible elongate body that extends proximally to distally (proximal/distal), and is configured to be inserted into a patient so that it extends around the target tissue, so that it can be bimanually pulled against the target tissue by applying tension to either end of the device. Thus, the device may be extended into, through, and/or around an intervertebral disc.

In some embodiments, the device may be configured to provide suction and or irrigation to aid in the modification and removal of the disc material. Alternatively, a suction/irrigation device separate from the tissue modification device may be used throughout the procedure. Furthermore, the devices may be powered devices, such as powered mechanical tissue modification devices. For example, the modification device may be a reciprocating saw, a band saw, a chain saw, or any other suitable powered mechanical tissue modification device.

For example, as shown in FIGS. 140A and 140B, the device may have an elongated ribbon shape that is long and flat with a width greater than the thickness, the device may include a first major surface (e.g., a front) and a second major surface (a back), and have edges (minor surfaces) between the first and second major surfaces. The first major surface may be referred to as the posterior or front surface and the second major surface may be referred to as the anterior or back surface. The devices described herein may be flexible along the anterior and posterior surfaces, and the posterior or front surface may include one or more cutting edges configured to cut tissue as the anterior surface of the device is urged against a tissue. The anterior surface may be configured to shield or protect non-target tissue. In some embodiments, the edges or minor surfaces may include one or more cutting edges configured to cut tissue as the edge the device is urged against a tissue. In particular, the cutting edges on the minor surface may be configured to prepare (roughen and/or remove) the vertebral endplates for fusion. As shown in FIG. 140A, the tissue modification device may include a plurality of rungs thread over one or more cables running along the length of the device. As mentioned, in operation, the device is urged against the target tissue and may be moved in the proximal/distal direction to modify (e.g., cut) the target tissue. For example, both the proximal and distal ends of the tissue-modification device may be pulled to urge the device against the target tissue, and may each be alternately pulled to a greater degree than the other handle to slide the device over the target tissue, allowing the cutting edges to cut and modify the target tissue.

The device shown in FIGS. 141A and 141B is similar to that described above with references to FIGS. 140A and 140B, however the device, as shown, has a C shaped cross-section. A cross section of this shape may aid in collecting and removing tissue as the device is advanced through the disc. In some embodiments, as described above, the device may include suction and/or irrigation. For example, the device may have aperture(s) along the length of the device configured for suction and/or irrigation. Suction may provided the added benefit of clearing tissue from the blades such that they can continue to cut additional tissue. As shown, the device includes blades on multiple surfaces of the device. Alternatively, the device may include blades on only a single surface or any other suitable combination of surfaces.

Alternatively, in FIGS. 142A and 142B, the tissue modification device may include a cutting edge 14200 coupled to a tissue capture device 14201. For example, as shown, the cutting edge may be ring shaped, either circular or elliptical, for example. As the device is advanced through the disc, the cutting edge may cut disc material. The tissue capture device may function as a net, for example, trailing behind the cutting edge and configured to capture and remove tissue cut by the cutting edge.

As shown in FIGS. 143A and 143B, the tissue modification device may be a brush like or “pipe-cleaner” like device having a plurality of bristles. As the device is pulled though the disc, the bristles may function to scrape or cut disc material, while collecting the removed material within the bristles such that the material may be removed from the disc. In some embodiments, the bristles may be flexible such that they may flex and bend while be inserted through a small access location and then expand again once within the disc.

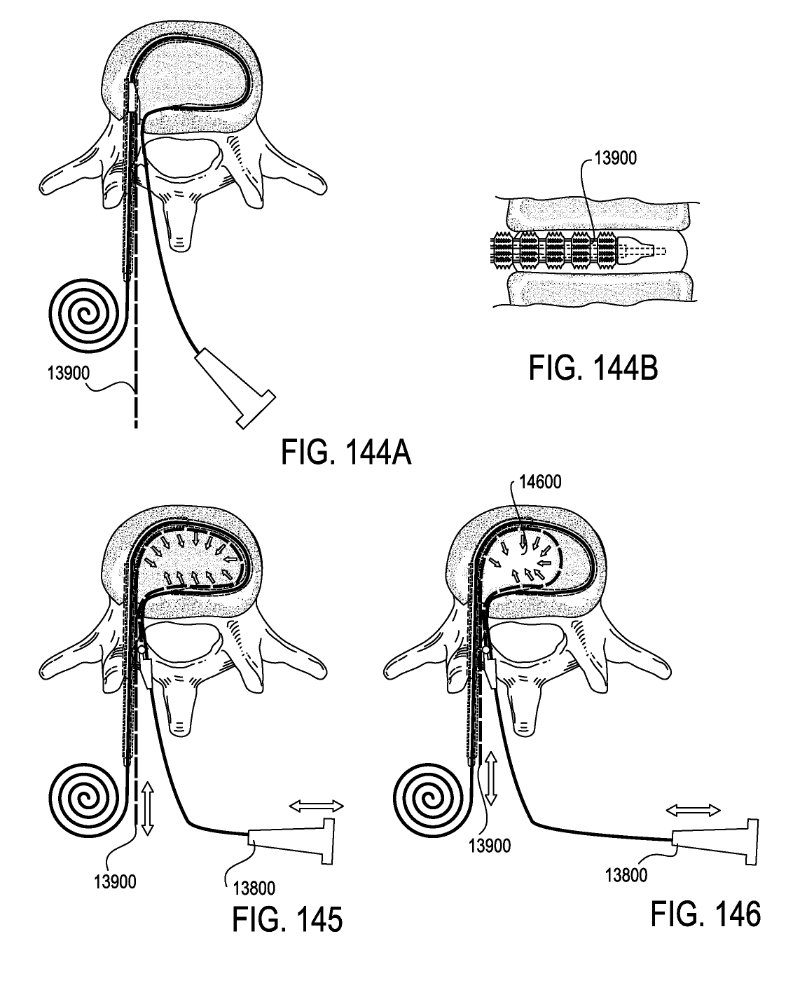

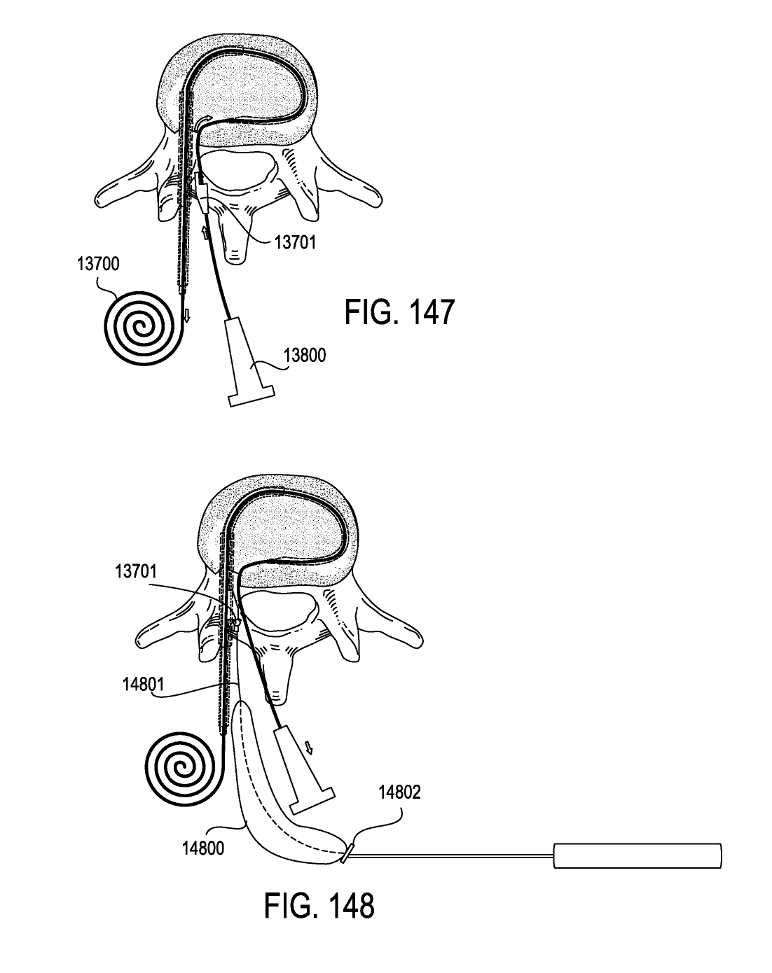

As shown in FIGS. 144A and 144B, once the device 13900 is coupled to the guidewire, it may be advanced into the disc along the curved path. As described above, the device may be advanced along the outside of the probe and over tube, while the guidewire is advanced within the probe and over tube. As shown in FIG. 144B, the cutting edges on the posterior or front side of the device may be positioned to modify the nucleus disc material, while the cutting edges on the side or minor surfaces may be positioned to modify the articular cartilage or vertebral end plates. As shown in FIG. 145, the device may be advanced into the disc by pulling on the distal end of the guidewire and/or distal handle. Once the device is advanced such that it substantially surrounds the disc nucleus, the proximal end of the device (or guidewire) and the distal end of the device (and/or guidewire) may be reciprocated back and forth such that the device moves within the disc to modify the target tissue 14600. While the device is reciprocated back and forth, the device may be pulled posteriorly toward the access location/opening in the disc annulus, as shown in FIG. 146. The device may be pulled posteriorly and out of the disc such that it modifies and removed disc tissue along its path. In some embodiments, the device may be reciprocated and then pulled posteriorly in separate steps. The device may be pulled posteriorly a small amount, reciprocated again, and then pulled posteriorly again.

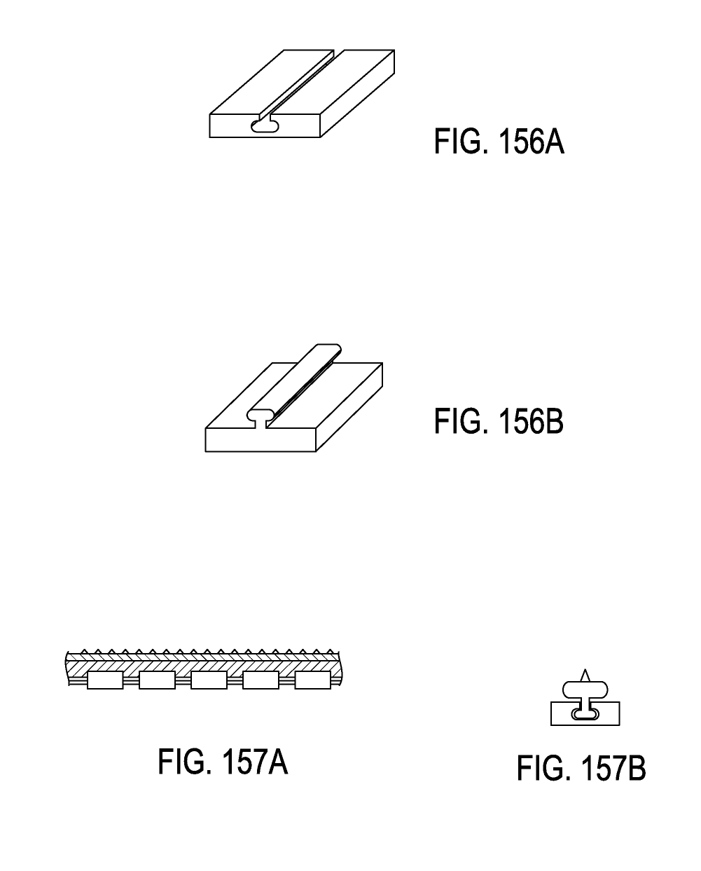

Furthermore, as shown in FIG. 146, once the device is advanced to a certain location within the disc, it may be desirable to replace the device back in the anterior portion of the disc (i.e. the starting position). This may be accomplished in one of several variations. For example, as shown in FIG. 155, additional instruments may be advanced over the guidewire and/or proximal handle of the tissue modification device and may then apply pressure to the tissue modification device to push the device back to the anterior portion of the disc. In some embodiments, the instruments are preferably rigid in at least one direction such that they may apply a force to the tissue modification device. Alternatively, the device may be coupled to the guidewire with a spring or other spring loaded coupler. For example, as the tissue modification device is pulled posteriorly in the disc, the spring may become loaded. Once the tissue modification is released and/or less force is applied in the posterior direction, the spring will return the device to the anterior portion of the disc. As described in more detail below with respect to FIGS. 156A-157B, the spring may be the rail or other connector coupling the tissue modification device to the distractor device.

As shown in FIG. 147, in some embodiments, once the device has been pulled entirely out of the disc, the device may be removed from the guidewire hitch. Alternatively, the device may be pulled back through the disc by pulling on the proximal end of the wire and may be removed from the guidewire in that orientation. In some embodiments, the device may be removed and a second device may be attached to the guidewire. For example, devices of different sizes may be sequentially attached to the guidewire and used to modify tissue. Alternatively, devices with different functions may be attached to the guidewire sequentially. For example, a tissue cutting device may first be used within the disc. Second, a tissue collection device may be coupled to the guidewire and used within the disc. In some embodiments, multiple devices may be coupled to a single guidewire and used in parallel or sequentially.

As shown in FIG. 148, once the target tissue (e.g. disc material) has been removed, and the modification and/or collection device has been removed, an implant 14800 may be coupled to the guidewire and pulled into position within the disc. The implant may be a banana shaped implant as shown, or may alternatively have any other suitable shape or configuration. As described below with reference to FIG. 152C, the implant may include multiple pieces that can be coupled to one another once implanted within the disc space. As shown, the implant may be coupled to a tether 14801. For example, the implant may include a lumen through which the tether may be placed. The tether may be configured to couple to the guidewire hitch. In some embodiments, the implant may also be coupled to a proximal handle. As shown, the tether and/or handle include a stop 14802, such that as the implant is pulled into position, it cannot slide past the stop. Additionally, the stop may function to push the implant from the proximal direction. In some embodiments, the implant may be coupled directly to the guidewire hitch and/or the tether may be coupled to the implant in an end-to-end configuration.

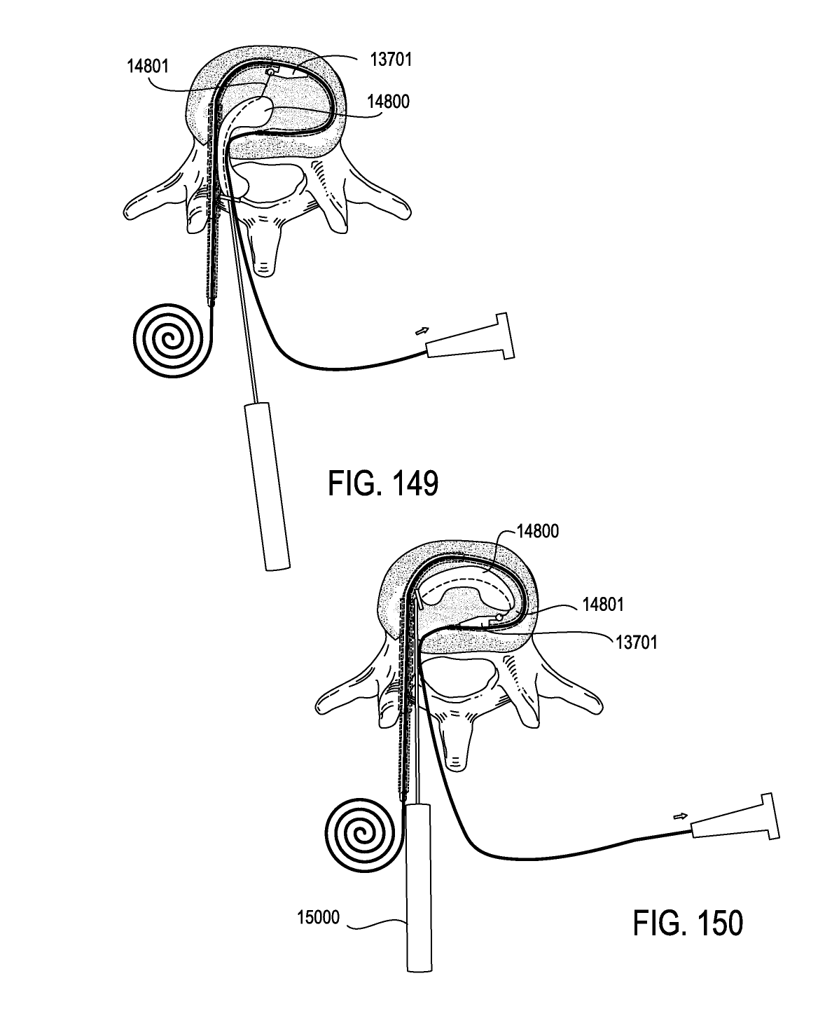

As shown in FIG. 149, as the guidewire is pulled and advanced through the probe, the implant is pulled into the disc. In some embodiments, the length of the tether may contribute to controlling the position of the implant within the disc. For example, a shorter tether will pull the implant more anteriorly into the disc. This may be desirable in some fusion procedures. For example, some surgeons prefer to place an implant in the anterior rim of the interspace. This may prevent subsidence of the cage into the endplates. Conventionally, anterior placement of the implant was quite technically challenging and potentially dangerous, the devices and methods described herein provide a method of placing an implant in the anterior rim of the interspace with little to no risk to any great vessel. Alternatively, a longer tether will keep the implant more toward the posterior aspect of the disc interspace. To aid in pulling in and placing the implant within the disc, an impact slider may be coupled to the distal end of the guidewire, proximal to the distal handle. As the distal handle is pulled to pull the implant into position, the impact slider may be pulled up against the distal handle to provide an impact force to the distal handle (and guidewire) in the distal direction.

In some embodiments, if the implant is incorrectly positioned or incorrectly sized for example, the implant can be removed by simply pulling on the proximal end of the wire or proximal handle coupled to the implant.

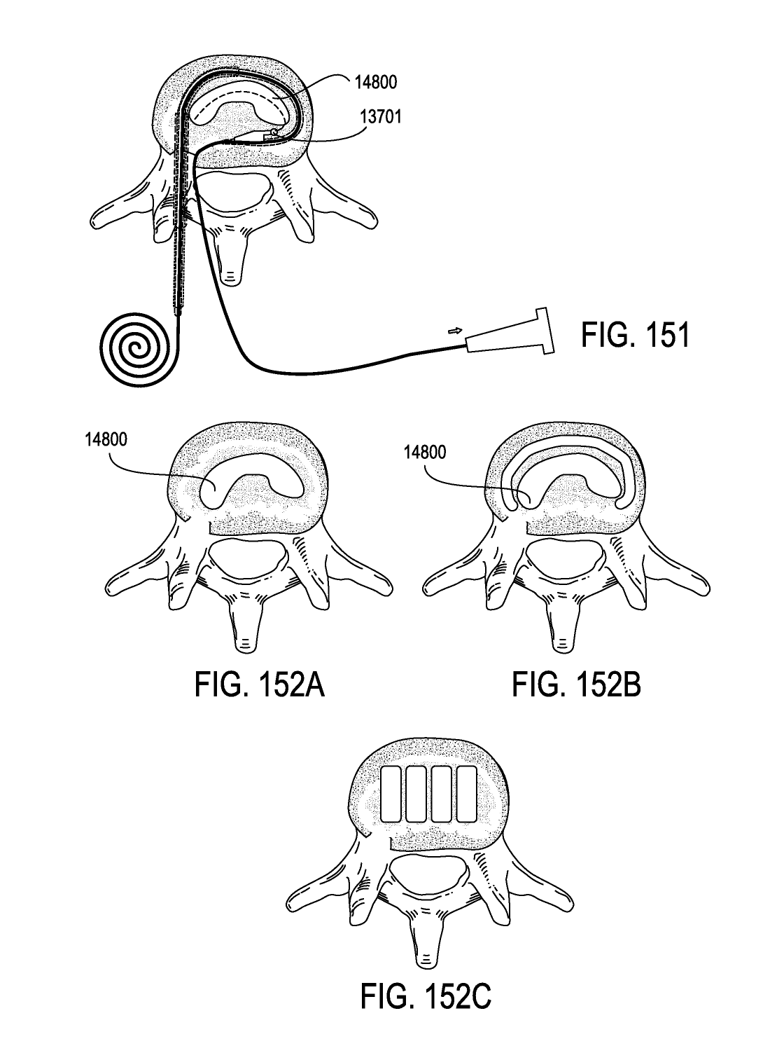

As shown in FIG. 150, once the implant is in the desired position within the disc space, the proximal handle may be removed from the implant. For example, the handle 15000 may be unscrewed or snapped off of the implant. Once the proximal handle is removed, the guidewire may be pulled distally further, such that the tether (coupled to the guidewire hitch) is pulled out of the implant, as shown in FIG. 151. Once the tether is removed from the implant, the tether may be removed from the guidewire hitch or alternatively, the wire and hitch together may be removed from the disc and from the patient. To remove the guidewire (and the tether) the wire may be pulled in either the proximal or the distal direction. The over tube and/or probe may also be removed at this time, leaving the implant in place, as shown in FIG. 152A.

As shown in FIG. 152B, before removing the guidewire, probe, etc., a graft sleeve or other suitable implant may be placed within the disc using the system described herein. As shown in FIG. 152C, the implant may include multiple pieces that may be coupled together to form a single implant having a larger footprint. Alternatively, multiple separate implants may be implanted with the system described herein.

In this embodiment, the distractor device or other first device pulled in by the guidewire, may include a rail (as shown in FIG. 156B) or a groove (as shown in FIG. 156A). As shown in FIGS. 157A and 157B, subsequent devices may be coupled to the rail or groove of the distractor device, and pulled in along the rail or groove. This rail or groove may replace the hitch of the guidewire, as described above.

The examples and illustrations included herein show, by way of illustration and not of limitation, specific embodiments in which the subject matter may be practiced. Other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An interspinous fusion system, the system comprising:
a first fixation plate configured to couple to a first lateral side of a spinous process;
a rod extending from the first fixation plate at a joint such that the rod is pivotable with respect to the first fixation plate; and
a second fixation plate configured to couple to a second lateral side of a spinous process opposite from the first fixation plate, wherein the second fixation plate is configured to couple to the rod such that the first and second fixation plates are compressed about the spinous process.

2. The system of claim 1, wherein the first fixation plate further comprises a slot sized and configured to receive a portion of the rod when the rod is pivoted with respect to the fixation plate such that the rod is not perpendicular to the plate.

3. The system of claim 1, wherein the second fixation plate is configured to be deployed from a first lateral side of a spinous process and to couple to the first lateral side of a spinous process and wherein the first fixation plate is configured to be deployed from the first lateral side of a spinous process and to couple to the second opposite lateral side of the spinous process.

4. The system of claim 1, wherein at least one of the first and the second fixation plate further comprises a coupling member disposed on the inside face of the fixation plate, wherein the coupling member of the inside face are configured to couple to the first or second lateral side of the spinous process.

5. The system of claim 4, wherein the coupling member comprises at least one protrusion sized and configured to dig into or catch onto bone thereby aiding in the coupling of the fixation plate to the spinous process.

6. The system of claim 1, wherein the second fixation plate further comprises a locking member configured to lock the second fixation plate onto the rod.

7. The system of claim 1, wherein the rod comprises a non-circular cross section.

8. The system of claim 1, wherein the rod is threaded along a portion of its length.

9. The system of claim 8, wherein the rod comprises both internal and external threads along a portion of its length.

10. The system of claim 1, wherein the joint is configured such that the rod is slidable with respect to the first fixation plate.

11. The system of claim 1, wherein the rod is pivotable with respect to the first fixation plate such that the rod may be positioned into an introduction position such that the angle between the fixation plate and the rod is less than 90 degrees, wherein the introduction position allows the first fixation plate to be deployed from the first lateral side of a spinous process and to couple to the second opposite lateral side of the spinous process.

12. The system of claim 11, wherein the angle between the fixation plate and the rod in the introduction position is about 0 degrees.

13. The system of claim 1, wherein the rod is pivotable with respect to the first fixation plate such that the rod may be positioned into a locking position having an angle between the fixation plate and the rod about equal to 90 degrees.

14. The system of claim 1, further comprising a handle configured to couple to the rod.

15. The system of claim 14, wherein the handle comprises an external handle portion and an internal handle portion disposed within the external handle portion, and wherein the external handle portion couples to external threads of the rod and the internal handle portion couples to internal threads of the rod.

16. The system of claim 15, wherein a distal end of the internal handle portion further comprises a locking portion, sized and configured to couple to the joint between the plate and the rod and to lock the plate position with respect to the rod.

17. The system of claim 15, wherein the external handle portion is configured to couple to a non-circular cross section of the rod.

18. The system of claim 1, further comprising a barrel sized and configured to be positioned over the rod and between two adjacent spinous processes.

19. The system of claim 18, wherein the barrel is sized and configured to be positioned over the rod such that the barrel will not rotate about the rod.

20. The system of claim 18, wherein the barrel is configured to receive graft material.

21. The system of claim 20, wherein the barrel comprises a window sized and configured to allow graft material, disposed within the barrel, to contact an adjacent spinous process.

22. The system of claim 18, wherein the barrel is further sized and configured to distract two adjacent spinous processes.

23. The system of claim 18, wherein the barrel is configured to lock the first fixation plate in position with respect to the rod.

24. The system of claim 23, wherein the barrel comprises a tab that extends from the end of the barrel and is sized and configured to couple to a slot in the first fixation plate such that the barrel locks the first fixation plate in position with respect to the rod.

25. The system of claim 18, wherein the barrel further comprises a rod locking mechanism configured to lock the barrel onto the rod.

26. The system of claim 1, wherein at least one of the first and second fixation plates further comprise a tapered leading edge configured to dissect tissue from a lateral side of the spinous process.

27. A method of performing an interspinous fusion unilaterally, the method comprising:
placing a first fixation plate, having a rod extending from the fixation plate, between two adjacent spinous processes from a fist lateral side of the spinous processes;
pivoting the rod with respect to the first fixation plate such that the plate abuts the second, opposite, lateral side of at least one of the spinous processes;
placing a second fixation plate such that it abuts the fist lateral side of at least one of the spinous processes from the fist lateral side of the spinous processes; and
coupling the second fixation plate to the rod.

28. The method of claim 27, wherein pivoting the rod further comprises sliding the rod with respect to the first fixation plate.

29. The method of claim 27, wherein coupling the second the fixation plate to the rod further comprises coupling the second the fixation plate to the rod such that the first and second fixation plates are compressed about the spinous process.

30. The method of claim 27, wherein coupling the second the fixation plate to the rod further comprises locking the second fixation plate onto the rod.

31. The method of claim 27, wherein placing the first fixation plate further comprises positioning the rod into an introduction position with respect to the first fixation plate such that the angle between the fixation plate and the rod is less than 90 degrees.

32. The method of claim 31, wherein the angle between the fixation plate and the rod is about 0 degrees.

33. The method of claim 27, wherein placing the first fixation plate further comprises positioning the rod into a locking position with respect to the first fixation plate such that the angle between the fixation plate and the rod is about equal to 90 degrees.

34. The method of claim 27, further comprising coupling a handle to the rod.

35. The method of claim 34, wherein coupling a handle to the rod further comprises coupling an external handle portion to external threads of the rod and coupling an internal handle portion to internal threads of the rod.

36. The method of claim 34, wherein coupling a handle to the rod further comprises locking the rod in position with respect to the first fixation plate with a locking portion of an internal handle portion.

37. The method of claim 27, further comprising positioning a barrel over the rod and between the two adjacent spinous processes.

38. The method of claim 37, further comprising placing graft material within the barrel.

39. The method of claim 37, wherein positioning the barrel over the rod further comprises distracting two adjacent spinous processes with the barrel.

40. The method of claim 37, wherein positioning the barrel over the rod further comprises locking the first fixation plate in position with respect to the rod with the barrel.

41. The method of claim 27, further comprising dissecting tissue from the first or second lateral side of a spinous process with a tapered edge of the first or second fixation plate.
